SEQUENCE LISTING (590 sequences)

SEQ ID NO: 1 - protein, 35 aa, Human immunodeficiency virus 1
CTRPNNNTRKSIHIGPGRAFYTTGEIIGDIRQAHC

SEQ ID NO: 2 - protein, 849 aa, Human immunodeficiency virus 1
MKVMGTKKNYQHLWRWGIMLLGMLMMSSAAEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGYSPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 3 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 4 - protein, 511 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 5 - protein, 475 aa, Human immunodeficiency virus 1
AEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKR

SEQ ID NO: 6 - protein, 481 aa, Human immunodeficiency virus 1
TEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 7 - protein, 5 aa, synthetic construct
DSYWS

SEQ ID NO: 8 - protein, 16 aa, synthetic construct
YVHKSGDTNYSPSLKS

SEQ ID NO: 9 - protein, 24 aa, synthetic construct
TLHGRRIYGIVAFNEWFTYFYMDV

SEQ ID NO: 10 - protein, 11 aa, synthetic construct
GEKSLGSRAVQ

SEQ ID NO: 11 - protein, 7 aa, synthetic construct
NNQDRPS

SEQ ID NO: 12 - protein, 12 aa, synthetic construct
HIWDSRVPTKWV

SEQ ID NO: 13 - protein, 16 aa, synthetic construct
YVHKSGDTNYNPSLKS

SEQ ID NO: 14 - protein, 5 aa, synthetic construct
NYYWT

SEQ ID NO: 15 - protein, 16 aa, synthetic construct
YISDRESATYNPSLNS

SEQ ID NO: 16 - protein, 24 aa, synthetic construct
ARRGQRIYGVVSFGEFFYYYSMDV

SEQ ID NO: 17 - protein, 11 aa, synthetic construct
GRQALGSRAVQ

SEQ ID NO: 18 - protein, 12 aa, synthetic construct
HMWDSRSGFSWS

SEQ ID NO: 19 - protein, 16 aa, synthetic construct
YISDRETTTYNPSLNS

SEQ ID NO: 20 - protein, 24 aa, synthetic construct
ARRGQRIYGVVSFGEFFYYYYMDV

SEQ ID NO: 21 - protein, 5 aa, synthetic construct
GRFWS

SEQ ID NO: 22 - protein, 16 aa, synthetic construct
YFSDTDRSEYNPSLRS

SEQ ID NO: 23 - protein, 24 aa, synthetic construct
AQQGKRIYGIVSFGEFFYYYYMDA

SEQ ID NO: 24 - protein, 11 aa, synthetic construct
GERSRGSRAVQ

SEQ ID NO: 25 - protein, 7 aa, synthetic construct
NNQDRPA

SEQ ID NO: 26 - protein, 12 aa, synthetic construct
HYWDSRSPISWI

SEQ ID NO: 27 - protein, 24 aa, synthetic construct
AQQGKRIYGIVSFGELFYYYYMDA

SEQ ID NO: 28 - protein, 5 aa, synthetic construct
DNYWS

SEQ ID NO: 29 - protein, 16 aa, synthetic construct
YVHDSGDTNYNPSLKS

SEQ ID NO: 30 - protein, 24 aa, synthetic construct
TKHGRRIYGVVAFKEWFTYFYMDV

SEQ ID NO: 31 - protein, 11 aa, synthetic construct
GEESLGSRSVI

SEQ ID NO: 32 - protein, 7 aa, synthetic construct
NNNDRPS

SEQ ID NO: 33 - protein, 12 aa, synthetic construct
HIWDSRRPTNWV

SEQ ID NO: 34 - protein, 5 aa, synthetic construct
DAYWS

SEQ ID NO: 35 - protein, 16 aa, synthetic construct
YVHHSGDTNYNPSLKR

SEQ ID NO: 36 - protein, 24 aa, synthetic construct
ALHGKRIYGIVALGELFTYFYMDV

SEQ ID NO: 37 - protein, 11 aa, synthetic construct
GKESIGSRAVQ

SEQ ID NO: 38 - protein, 12 aa, synthetic construct
HIYDARGGTNWV

SEQ ID NO: 39 - protein, 7 aa, synthetic construct
ACTYFWG

SEQ ID NO: 40 - protein, 22 aa, synthetic construct
SLSHCQSFWGSGWTFHNPSLKS

SEQ ID NO: 41 - protein, 19 aa, synthetic construct
FDGEVLVYNHWPKPAWVDL

SEQ ID NO: 42 - protein, 9 aa, synthetic construct
NGTATNFVS

SEQ ID NO: 43 - protein, 7 aa, synthetic construct
GVDKRPP

SEQ ID NO: 44 - protein, 10 aa, synthetic construct
GSLVGNWDVI

SEQ ID NO: 45 - protein, 7 aa, synthetic construct
ACDYFWG

SEQ ID NO: 46 - protein, 22 aa, synthetic construct
GLSHCAGYYNTGWTYHNPSLKS

SEQ ID NO: 47 - protein, 19 aa, synthetic construct
FDGEVLVYHDWPKPAWVDL

SEQ ID NO: 48 - protein, 9 aa, synthetic construct
TGTSNRFVS

SEQ ID NO: 49 - protein, 7 aa, synthetic construct
GVNKRPS

SEQ ID NO: 50 - protein, 10 aa, synthetic construct
SSLVGNWDVI

SEQ ID NO: 51 - protein, 22 aa, synthetic construct
SLSHCAGYYNSGWTYHNPSLKS

SEQ ID NO: 52 - protein, 19 aa, synthetic construct
FGGDVLVYHDWPKPAWVDL

SEQ ID NO: 53 - protein, 9 aa, synthetic construct
TGNINNFVS

SEQ ID NO: 54 - protein, 10 aa, synthetic construct
GSLAGNWDVV

SEQ ID NO: 55 - protein, 7 aa, synthetic construct
ACNSFWG

SEQ ID NO: 56 - protein, 22 aa, synthetic construct
SLSHCASYWNRGWTYHNPSLKS

SEQ ID NO: 57 - protein, 19 aa, synthetic construct
FGGEVLRYTDWPKPAWVDL

SEQ ID NO: 58 - protein, 9 aa, synthetic construct
TGTSNNFVS

SEQ ID NO: 59 - protein, 7 aa, synthetic construct
DVNKRPS

SEQ ID NO: 60 - protein, 7 aa, synthetic construct
GCDYFWG

SEQ ID NO: 61 - protein, 19 aa, synthetic construct
FDGEVLVYNDWPKPAWVDL

SEQ ID NO: 62 - protein, 7 aa, synthetic construct
TGHYYWG

SEQ ID NO: 63 - protein, 16 aa, synthetic construct
HIHYTTAVLHNPSLKS

SEQ ID NO: 64 - protein, 19 aa, synthetic construct
SGGDILYYYEWQKPHWFSP

SEQ ID NO: 65 - protein, 14 aa, synthetic construct
NGTSSDIGGWNFVS

SEQ ID NO: 66 - protein, 7 aa, synthetic construct
EVNKRPS

SEQ ID NO: 67 - protein, 10 aa, synthetic construct
SSLFGRWDVV

SEQ ID NO: 68 - protein, 12 aa, synthetic construct
GTDWGENDFHYG

SEQ ID NO: 69 - protein, 17 aa, synthetic construct
SIHWRGRTTHYKTSFRS

SEQ ID NO: 70 - protein, 18 aa, synthetic construct
HKYHDIFRVVPVAGWFDP

SEQ ID NO: 71 - protein, 11 aa, synthetic construct
RASQNVKNNLA

SEQ ID NO: 72 - protein, 7 aa, synthetic construct
DASSRAG

SEQ ID NO: 73 - protein, 9 aa, synthetic construct
QQYEEWPRT

SEQ ID NO: 74 - protein, 12 aa, synthetic construct
GGEWGDSDYHWG

SEQ ID NO: 75 - protein, 16 aa, synthetic construct
SIHWRGTTHYNAPFRG

SEQ ID NO: 76 - protein, 18 aa, synthetic construct
HKYHDIVMVVPIAGWFDP

SEQ ID NO: 77 - protein, 11 aa, synthetic construct
RASQSVKNNLA

SEQ ID NO: 78 - protein, 7 aa, synthetic construct
DTSSRAS

SEQ ID NO: 79 - protein, 12 aa, synthetic construct
GGEWGDKDYHWG

SEQ ID NO: 80 - protein, 16 aa, synthetic construct
SIHWRGTTHYKESLRR

SEQ ID NO: 81 - protein, 18 aa, synthetic construct
HRHHDVFMLVPIAGWFDV

SEQ ID NO: 82 - protein, 11 aa, synthetic construct
RASQNINKNLA

SEQ ID NO: 83 - protein, 7 aa, synthetic construct
ETYSKIA

SEQ ID NO: 84 - protein, 6 aa, synthetic construct
SDHSWT

SEQ ID NO: 85 - protein, 16 aa, synthetic construct
DIHYNGATTYNPSLRS

SEQ ID NO: 86 - protein, 21 aa, synthetic construct
NAIRIYGVVALGEWFHYGMDV

SEQ ID NO: 87 - protein, 11 aa, synthetic construct
SGAPLTSRFTY

SEQ ID NO: 88 - protein, 7 aa, synthetic construct
RSSQRSS

SEQ ID NO: 89 - protein, 11 aa, synthetic construct
QSSDTSDSYKM

SEQ ID NO: 90 - protein, 16 aa, synthetic construct
DVHYNGDNTYNPSLRG

SEQ ID NO: 91 - protein, 21 aa, synthetic construct
NVIRVFGVISLGEWFHYGMDV

SEQ ID NO: 92 - protein, 11 aa, synthetic construct
SGPPLASRYTY

SEQ ID NO: 93 - protein, 7 aa, synthetic construct
RDRQFPS

SEQ ID NO: 94 - protein, 16 aa, synthetic construct
DVHYNGDTTYNPSLRG

SEQ ID NO: 95 - protein, 11 aa, synthetic construct
SGAALTSRFTY

SEQ ID NO: 96 - protein, 7 aa, synthetic construct
RTSQRSS

SEQ ID NO: 97 - protein, 16 aa, synthetic construct
DIHYGGDITYNPSLRS

SEQ ID NO: 98 - protein, 21 aa, synthetic construct
NVIRVFGVIALGEWFHYGMDV

SEQ ID NO: 99 - protein, 11 aa, synthetic construct
SGPPLASRYCY

SEQ ID NO: 100 - protein, 7 aa, synthetic construct
RDRQFSS

SEQ ID NO: 101 - protein, 11 aa, synthetic construct
QSSDINDSYKM

SEQ ID NO: 102 - protein, 11 aa, synthetic construct
QSSDTSDSFKM

SEQ ID NO: 103 - protein, 11 aa, synthetic construct
SGPPLATRYCY

SEQ ID NO: 104 - protein, 16 aa, synthetic construct
DIHYNGDKTYNPSLRG

SEQ ID NO: 105 - protein, 11 aa, synthetic construct
QSSDNSDSFKM

SEQ ID NO: 106 - protein, 5 aa, synthetic construct
DYAMA

SEQ ID NO: 107 - protein, 19 aa, synthetic construct
FMRGWAYGGSAQFAAFAVG

SEQ ID NO: 108 - protein, 22 aa, synthetic construct
EQRNKDYRYGQEGFGYSYGMDV

SEQ ID NO: 109 - protein, 11 aa, synthetic construct
RASHFIANYVN

SEQ ID NO: 110 - protein, 7 aa, synthetic construct
ESSTLQR

SEQ ID NO: 111 - protein, 9 aa, synthetic construct
QQSHSPPVT

SEQ ID NO: 112 - protein, 19 aa, synthetic construct
FIRGWAYGQAAQYGKSASG

SEQ ID NO: 113 - protein, 22 aa, synthetic construct
EQRGGDGRYSGDGFGYPYGMDV

SEQ ID NO: 114 - protein, 7 aa, synthetic construct
QSWTLNR

SEQ ID NO: 115 - protein, 9 aa, synthetic construct
QQSHSPPLS

SEQ ID NO: 116 - protein, 19 aa, synthetic construct
FIRGWAYGQSAQYGKSASG

SEQ ID NO: 117 - protein, 22 aa, synthetic construct
EQRGANGRYGGDGFGYSYGMDV

SEQ ID NO: 118 - protein, 7 aa, synthetic construct
ESSTLNR

SEQ ID NO: 119 - protein, 9 aa, synthetic construct
QQSHSPPVS

SEQ ID NO: 120 - protein, 6 aa, synthetic construct
GASISD

SEQ ID NO: 121 - protein, 9 aa, synthetic construct
YVHKSGDTN

SEQ ID NO: 122 - protein, 6 aa, synthetic construct
GDSMNN

SEQ ID NO: 123 - protein, 9 aa, synthetic construct
YISDRESAT

SEQ ID NO: 124 - protein, 6 aa, synthetic construct
GGSISN

SEQ ID NO: 125 - protein, 9 aa, synthetic construct
YISDRETTT

SEQ ID NO: 126 - protein, 6 aa, synthetic construct
NGSVSG

SEQ ID NO: 127 - protein, 9 aa, synthetic construct
YFSDTDRSE

SEQ ID NO: 128 - protein, 6 aa, synthetic construct
GTLVRD

SEQ ID NO: 129 - protein, 9 aa, synthetic construct
YVHDSGDTN

SEQ ID NO: 130 - protein, 6 aa, synthetic construct
GASIND

SEQ ID NO: 131 - protein, 9 aa, synthetic construct
YVHHSGDTN

SEQ ID NO: 132 - protein, 8 aa, synthetic construct
GESTGACT

SEQ ID NO: 133 - protein, 15 aa, synthetic construct
SLSHCQSFWGSGWTF

SEQ ID NO: 134 - protein, 8 aa, synthetic construct
GDSTAACD

SEQ ID NO: 135 - protein, 15 aa, synthetic construct
GLSHCAGYYNTGWTY

SEQ ID NO: 136 - protein, 15 aa, synthetic construct
SLSHCAGYYNSGWTY

SEQ ID NO: 137 - protein, 8 aa, synthetic construct
GDSTAACN

SEQ ID NO: 138 - protein, 8 aa, synthetic construct
GDSTAGCD

SEQ ID NO: 139 - protein, 8 aa, synthetic construct
GESINTGH

SEQ ID NO: 140 - protein, 9 aa, synthetic construct
HIHYTTAVL

SEQ ID NO: 141 - protein, 13 aa, synthetic construct
GGSMRGTDWGEND

SEQ ID NO: 142 - protein, 10 aa, synthetic construct
SIHWRGRTTH

SEQ ID NO: 143 - protein, 13 aa, synthetic construct
GGSIRGGEWGDSD

SEQ ID NO: 144 - protein, 9 aa, synthetic construct
SIHWRGTTH

SEQ ID NO: 145 - protein, 13 aa, synthetic construct
GDSIRGGEWGDKD

SEQ ID NO: 146 - protein, 8 aa, synthetic construct
QDSRPSDH

SEQ ID NO: 147 - protein, 5 aa, synthetic construct
HYNGA

SEQ ID NO: 148 - protein, 8 aa, synthetic construct
NDSRPSDH

SEQ ID NO: 149 - protein, 8 aa, synthetic construct
GDSRPSDH

SEQ ID NO: 150 - protein, 5 aa, synthetic construct
HYNGD

SEQ ID NO: 151 - protein, 5 aa, synthetic construct
HYGGD

SEQ ID NO: 152 - protein, 7 aa, synthetic construct
GFYFPDY

SEQ ID NO: 153 - protein, 8 aa, synthetic construct
RGWAYGGS

SEQ ID NO: 154 - protein, 7 aa, synthetic construct
DFYFPDY

SEQ ID NO: 155 - protein, 8 aa, synthetic construct
RGWAYGQA

SEQ ID NO: 156 - protein, 8 aa, synthetic construct
RGWAYGQS

SEQ ID NO: 157 - protein, 8 aa, synthetic construct
GASISDSY

SEQ ID NO: 158 - protein, 7 aa, synthetic construct
VHKSGDT

SEQ ID NO: 159 - protein, 26 aa, synthetic construct
ARTLHGRRIYGIVAFNEWFTYFYMDV

SEQ ID NO: 160 - protein, 6 aa, synthetic construct
SLGSRA

SEQ ID NO: 161 - skipped

SEQ ID NO: 162 - protein, 8 aa, synthetic construct
GDSMNNYY

SEQ ID NO: 163 - protein, 7 aa, synthetic construct
ISDRESA

SEQ ID NO: 164 - protein, 26 aa, synthetic construct
ATARRGQRIYGVVSFGEFFYYYSMDV

SEQ ID NO: 165 - protein, 6 aa, synthetic construct
ALGSRA

SEQ ID NO: 166 - protein, 26 aa, synthetic construct
ARARRGQRIYGVVSFGEFFYYYSMDV

SEQ ID NO: 167 - protein, 8 aa, synthetic construct
GGSISNYY

SEQ ID NO: 168 - protein, 7 aa, synthetic construct
ISDRETT

SEQ ID NO: 169 - protein, 26 aa, synthetic construct
ATARRGQRIYGVVSFGEFFYYYYMDV

SEQ ID NO: 170 - protein, 8 aa, synthetic construct
NGSVSGRF

SEQ ID NO: 171 - protein, 7 aa, synthetic construct
FSDTDRS

SEQ ID NO: 172 - protein, 26 aa, synthetic construct
ARAQQGKRIYGIVSFGELFYYYYMDA

SEQ ID NO: 173 - protein, 6 aa, synthetic construct
SRGSRA

SEQ ID NO: 174 - protein, 26 aa, synthetic construct
ARAQQGKRIYGIVSFGEFFYYYYMDA

SEQ ID NO: 175 - protein, 8 aa, synthetic construct
GTLVRDNY

SEQ ID NO: 176 - protein, 7 aa, synthetic construct
VHDSGDT

SEQ ID NO: 177 - protein, 26 aa, synthetic construct
ATTKHGRRIYGVVAFKEWFTYFYMDV

SEQ ID NO: 178 - protein, 6 aa, synthetic construct
SIGSRA

SEQ ID NO: 179 - protein, 8 aa, synthetic construct
GASINDAY

SEQ ID NO: 180 - protein, 7 aa, synthetic construct
VHHSGDT

SEQ ID NO: 181 - protein, 26 aa, synthetic construct
ARALHGKRIYGIVALGELFTYFYMDV

SEQ ID NO: 182 - protein, 6 aa, synthetic construct
SLGSRS

SEQ ID NO: 183 - skipped

SEQ ID NO: 184 - protein, 10 aa, synthetic construct
GESTGACTYF

SEQ ID NO: 185 - protein, 13 aa, synthetic construct
LSHCQSFWGSGWT

SEQ ID NO: 186 - protein, 21 aa, synthetic construct
ARFDGEVLVYNHWPKPAWVDL

SEQ ID NO: 187 - protein, 4 aa, synthetic construct
ATNF

SEQ ID NO: 188 - skipped

SEQ ID NO: 189 - protein, 10 aa, synthetic construct
GDSTAACDYF

SEQ ID NO: 190 - protein, 13 aa, synthetic construct
LSHCAGYYNTGWT

SEQ ID NO: 191 - protein, 21 aa, synthetic construct
ARFDGEVLVYHDWPKPAWVDL

SEQ ID NO: 192 - protein, 4 aa, synthetic construct
SNRF

SEQ ID NO: 193 - skipped

SEQ ID NO: 194 - protein, 13 aa, synthetic construct
LSHCAGYYNSGWT

SEQ ID NO: 195 - protein, 21 aa, synthetic construct
ARFGGDVLVYHDWPKPAWVDL

SEQ ID NO: 196 - protein, 4 aa, synthetic construct
INNF

SEQ ID NO: 197 - protein, 10 aa, synthetic construct
GDSTAACNSF

SEQ ID NO: 198 - protein, 13 aa, synthetic construct
LSHCASYWNRGWT

SEQ ID NO: 199 - protein, 21 aa, synthetic construct
ARFGGEVLRYTDWPKPAWVDL

SEQ ID NO: 200 - protein, 4 aa, synthetic construct
SNNF

SEQ ID NO: 201 - skipped

SEQ ID NO: 202 - protein, 10 aa, synthetic construct
GDSTAGCDYF

SEQ ID NO: 203 - protein, 13 aa, synthetic construct
LSHCAGYYNTGWT

SEQ ID NO: 204 - protein, 21 aa, synthetic construct
ARFDGEVLVYNDWPKPAWVDL

SEQ ID NO: 205 - protein, 10 aa, synthetic construct
GESINTGHYY

SEQ ID NO: 206 - protein, 7 aa, synthetic construct
IHYTTAV

SEQ ID NO: 207 - protein, 21 aa, synthetic construct
VRSGGDILYYYEWQKPHWFSP

SEQ ID NO: 208 - protein, 9 aa, synthetic construct
SSDIGGWNF

SEQ ID NO: 209 - skipped

SEQ ID NO: 210 - protein, 15 aa, synthetic construct
GGSMRGTDWGENDFH

SEQ ID NO: 211 - protein, 8 aa, synthetic construct
IHWRGRTT

SEQ ID NO: 212 - protein, 20 aa, synthetic construct
ARHKYHDIFRVVPVAGWFDP

SEQ ID NO: 213 - protein, 6 aa, synthetic construct
QNVKNN

SEQ ID NO: 214 - skipped

SEQ ID NO: 215 - protein, 15 aa, synthetic construct
GGSIRGGEWGDSDYH

SEQ ID NO: 216 - protein, 7 aa, synthetic construct
IHWRGTT

SEQ ID NO: 217 - protein, 20 aa, synthetic construct
VKHKYHDIVMVVPIAGWFDP

SEQ ID NO: 218 - protein, 6 aa, synthetic construct
QSVKNN

SEQ ID NO: 219 - skipped

SEQ ID NO: 220 - protein, 15 aa, synthetic construct
GDSIRGGEWGDKDYH

SEQ ID NO: 221 - protein, 20 aa, synthetic construct
ARHRHHDVFMLVPIAGWFDV

SEQ ID NO: 222 - protein, 6 aa, synthetic construct
QNINKN

SEQ ID NO: 223 - skipped

SEQ ID NO: 224 - protein, 9 aa, synthetic construct
QDSRPSDHS

SEQ ID NO: 225 - protein, 7 aa, synthetic construct
IHYNGAT

SEQ ID NO: 226 - protein, 6 aa, synthetic construct
PLTSRF

SEQ ID NO: 227 - skipped

SEQ ID NO: 228 - protein, 9 aa, synthetic construct
NDSRPSDHS

SEQ ID NO: 229 - protein, 9 aa, synthetic construct
GDSRPSDHS

SEQ ID NO: 230 - protein, 7 aa, synthetic construct
VHYNGDN

SEQ ID NO: 231 - protein, 6 aa, synthetic construct
PLASRY

SEQ ID NO: 232 - skipped

SEQ ID NO: 233 - protein, 7 aa, synthetic construct
VHYNGDT

SEQ ID NO: 234 - protein, 6 aa, synthetic construct
ALTSRF

SEQ ID NO: 235 - skipped

SEQ ID NO: 236 - protein, 7 aa, synthetic construct
IHYGGDI

SEQ ID NO: 237 - protein, 6 aa, synthetic construct
PLATRY

SEQ ID NO: 238 - protein, 7 aa, synthetic construct
IHYNGDK

SEQ ID NO: 239 - protein, 8 aa, synthetic construct
GFYFPDYA

SEQ ID NO: 240 - protein, 10 aa, synthetic construct
MRGWAYGGSA

SEQ ID NO: 241 - protein, 6 aa, synthetic construct
HFIANY

SEQ ID NO: 242 - skipped

SEQ ID NO: 243 - protein, 8 aa, synthetic construct
DFYFPDYA

SEQ ID NO: 244 - protein, 10 aa, synthetic construct
IRGWAYGQAA

SEQ ID NO: 245 - skipped

SEQ ID NO: 246 - protein, 10 aa, synthetic construct
IRGWAYGQSA

SEQ ID NO: 247 - protein, 10 aa, synthetic construct
VSGASISDSY

SEQ ID NO: 248 - protein, 16 aa, synthetic construct
VHKSGDTNYSPSLKSR

SEQ ID NO: 249 - protein, 23 aa, synthetic construct
TLHGRRIYGIVAFNEWFTYFYMD

SEQ ID NO: 250 - protein, 8 aa, synthetic construct
EKSLGSRA

SEQ ID NO: 251 - protein, 12 aa, synthetic construct
NNQDRPSGIPER

SEQ ID NO: 252 - protein, 9 aa, synthetic construct
WDSRVPTKW

SEQ ID NO: 253 - protein, 16 aa, synthetic construct
VHKSGDTNYNPSLKSR

SEQ ID NO: 254 - protein, 10 aa, synthetic construct
VSGDSMNNYY

SEQ ID NO: 255 - protein, 16 aa, synthetic construct
ISDRESATYNPSLNSR

SEQ ID NO: 256 - protein, 23 aa, synthetic construct
ARRGQRIYGVVSFGEFFYYYSMD

SEQ ID NO: 257 - protein, 8 aa, synthetic construct
RQALGSRA

SEQ ID NO: 258 - protein, 9 aa, synthetic construct
WDSRSGFSW

SEQ ID NO: 259 - protein, 10 aa, synthetic construct
VSGGSISNYY

SEQ ID NO: 260 - protein, 16 aa, synthetic construct
ISDRETTTYNPSLNSR

SEQ ID NO: 261 - protein, 23 aa, synthetic construct
ARRGQRIYGVVSFGEFFYYYYMD

SEQ ID NO: 262 - protein, 10 aa, synthetic construct
VSNGSVSGRF

SEQ ID NO: 263 - protein, 16 aa, synthetic construct
FSDTDRSEYNPSLRSR

SEQ ID NO: 264 - protein, 23 aa, synthetic construct
AQQGKRIYGIVSFGELFYYYYMD

SEQ ID NO: 265 - protein, 8 aa, synthetic construct
ERSRGSRA

SEQ ID NO: 266 - protein, 12 aa, synthetic construct
NNQDRPAGVSER

SEQ ID NO: 267 - protein, 9 aa, synthetic construct
WDSRSPISW

SEQ ID NO: 268 - protein, 23 aa, synthetic construct
AQQGKRIYGIVSFGEFFYYYYMD

SEQ ID NO: 269 - protein, 10 aa, synthetic construct
VSGASINDAY

SEQ ID NO: 270 - protein, 16 aa, synthetic construct
VHHSGDTNYNPSLKRR

SEQ ID NO: 271 - protein, 23 aa, synthetic construct
ALHGKRIYGIVALGELFTYFYMD

SEQ ID NO: 272 - protein, 8 aa, synthetic construct
KESIGSRA

SEQ ID NO: 273 - protein, 12 aa, synthetic construct
NNQDRPAGVPER

SEQ ID NO: 274 - protein, 9 aa, synthetic construct
YDARGGTNW

SEQ ID NO: 275 - protein, 10 aa, synthetic construct
VSGTLVRDNY

SEQ ID NO: 276 - protein, 16 aa, synthetic construct
VHDSGDTNYNPSLKSR

SEQ ID NO: 277 - protein, 23 aa, synthetic construct
TKHGRRIYGVVAFKEWFTYFYMD

SEQ ID NO: 278 - protein, 8 aa, synthetic construct
EESLGSRS

SEQ ID NO: 279 - protein, 12 aa, synthetic construct
NNNDRPSGIPDR

SEQ ID NO: 280 - protein, 9 aa, synthetic construct
WDSRRPTNW

SEQ ID NO: 281 - protein, 12 aa, synthetic construct
VSGESTGACTYF

SEQ ID NO: 282 - protein, 22 aa, synthetic construct
LSHCQSFWGSGWTFHNPSLKSR

SEQ ID NO: 283 - protein, 18 aa, synthetic construct
FDGEVLVYNHWPKPAWVD

SEQ ID NO: 284 - protein, 6 aa, synthetic construct
GTATNF

SEQ ID NO: 285 - protein, 12 aa, synthetic construct
GVDKRPPGVPDR

SEQ ID NO: 286 - protein, 7 aa, synthetic construct
LVGNWDV

SEQ ID NO: 287 - protein, 12 aa, synthetic construct
VSGDSTAACDYF

SEQ ID NO: 288 - protein, 22 aa, synthetic construct
LSHCAGYYNTGWTYHNPSLKSR

SEQ ID NO: 289 - protein, 18 aa, synthetic construct
FDGEVLVYHDWPKPAWVD

SEQ ID NO: 290 - protein, 6 aa, synthetic construct
GTSNRF

SEQ ID NO: 291 - protein, 12 aa, synthetic construct
GVNKRPSGVPDR

SEQ ID NO: 292 - protein, 22 aa, synthetic construct
LSHCAGYYNSGWTYHNPSLKSR

SEQ ID NO: 293 - protein, 18 aa, synthetic construct
FGGDVLVYHDWPKPAWVD

SEQ ID NO: 294 - protein, 6 aa, synthetic construct
GNINNF

SEQ ID NO: 295 - protein, 7 aa, synthetic construct
LAGNWDV

SEQ ID NO: 296 - protein, 12 aa, synthetic construct
VSGDSTAACNSF

SEQ ID NO: 297 - protein, 22 aa, synthetic construct
LSHCASYWNRGWTYHNPSLKSR

SEQ ID NO: 298 - protein, 18 aa, synthetic construct
FGGEVLRYTDWPKPAWVD

SEQ ID NO: 299 - protein, 6 aa, synthetic construct
GTSNNF

SEQ ID NO: 300 - protein, 12 aa, synthetic construct
DVNKRPSGVPDR

SEQ ID NO: 301 - protein, 12 aa, synthetic construct
VSGDSTAGCDYF

SEQ ID NO: 302 - protein, 18 aa, synthetic construct
FDGEVLVYNDWPKPAWVD

SEQ ID NO: 303 - protein, 12 aa, synthetic construct
VSGESINTGHYY

SEQ ID NO: 304 - protein, 16 aa, synthetic construct
IHYTTAVLHNPSLKSR

SEQ ID NO: 305 - protein, 18 aa, synthetic construct
SGGDILYYYEWQKPHWFS

SEQ ID NO: 306 - protein, 11 aa, synthetic construct
GTSSDIGGWNF

SEQ ID NO: 307 - protein, 12 aa, synthetic construct
EVNKRPSGVPGR

SEQ ID NO: 308 - protein, 7 aa, synthetic construct
LFGRWDV

SEQ ID NO: 309 - protein, 17 aa, synthetic construct
VSGGSMRGTDWGENDFH

SEQ ID NO: 310 - protein, 17 aa, synthetic construct
IHWRGRTTHYKTSFRSR

SEQ ID NO: 311 - protein, 17 aa, synthetic construct
HKYHDIFRVVPVAGWFD

SEQ ID NO: 312 - protein, 8 aa, synthetic construct
ASQNVKNN

SEQ ID NO: 313 - protein, 12 aa, synthetic construct
DASSRAGGIPDR

SEQ ID NO: 314 - protein, 6 aa, synthetic construct
YEEWPR

SEQ ID NO: 315 - protein, 17 aa, synthetic construct
ASGGSIRGGEWGDSDYH

SEQ ID NO: 316 - protein, 16 aa, synthetic construct
IHWRGTTHYNAPFRGR

SEQ ID NO: 317 - protein, 17 aa, synthetic construct
HKYHDIVMVVPIAGWFD

SEQ ID NO: 318 - protein, 8 aa, synthetic construct
ASQSVKNN

SEQ ID NO: 319 - protein, 12 aa, synthetic construct
DTSSRASGIPAR

SEQ ID NO: 320 - protein, 17 aa, synthetic construct
VSGDSIRGGEWGDKDYH

SEQ ID NO: 321 - protein, 16 aa, synthetic construct
IHWRGTTHYKESLRRR

SEQ ID NO: 322 - protein, 17 aa, synthetic construct
HRHHDVFMLVPIAGWFD

SEQ ID NO: 323 - protein, 8 aa, synthetic construct
ASQNINKN

SEQ ID NO: 324 - protein, 12 aa, synthetic construct
ETYSKIAAFPAR

SEQ ID NO: 325 - protein, 11 aa, synthetic construct
VSQDSRPSDHS

SEQ ID NO: 326 - protein, 16 aa, synthetic construct
IHYNGATTYNPSLRSR

SEQ ID NO: 327 - protein, 20 aa, synthetic construct
NAIRIYGVVALGEWFHYGMD

SEQ ID NO: 328 - protein, 8 aa, unidentified
GAPLTSRF

SEQ ID NO: 329 - protein, 12 aa, synthetic construct
RSSQRSSGWSGR

SEQ ID NO: 330 - protein, 8 aa, synthetic construct
SDTSDSYK

SEQ ID NO: 331 - protein, 11 aa, synthetic construct
VSNDSRPSDHS

SEQ ID NO: 332 - protein, 11 aa, synthetic construct
VFGDSRPSDHS

SEQ ID NO: 333 - protein, 16 aa, synthetic construct
VHYNGDNTYNPSLRGR

SEQ ID NO: 334 - protein, 20 aa, synthetic construct
NVIRVFGVISLGEWFHYGMD

SEQ ID NO: 335 - protein, 8 aa, synthetic construct
GPPLASRY

SEQ ID NO: 336 - protein, 12 aa, synthetic construct
RDRQFPSGVSGR

SEQ ID NO: 337 - protein, 16 aa, synthetic construct
VHYNGDTTYNPSLRGR

SEQ ID NO: 338 - protein, 8 aa, synthetic construct
GAALTSRF

SEQ ID NO: 339 - protein, 12 aa, synthetic construct
RTSQRSSGWSGR

SEQ ID NO: 340 - protein, 11 aa, synthetic construct
ISGDSRPSDHS

SEQ ID NO: 341 - protein, 16 aa, synthetic construct
IHYGGDITYNPSLRSR

SEQ ID NO: 342 - protein, 20 aa, synthetic construct
NVIRVFGVIALGEWFHYGMD

SEQ ID NO: 343 - protein, 12 aa, synthetic construct
RDRQFSSGMSGR

SEQ ID NO: 344 - protein, 8 aa, synthetic construct
SDINDSYK

SEQ ID NO: 345 - protein, 12 aa, synthetic construct
RDRQFSSGISGR

SEQ ID NO: 346 - protein, 8 aa, synthetic construct
SDTSDSFK

SEQ ID NO: 347 - protein, 8 aa, synthetic construct
GPPLATRY

SEQ ID NO: 348 - protein, 12 aa, synthetic construct
RDRQFSSGVSGR

SEQ ID NO: 349 - protein, 16 aa, synthetic construct
IHYNGDKTYNPSLRGR

SEQ ID NO: 350 - protein, 8 aa, synthetic construct
SDNSDSFK

SEQ ID NO: 351 - protein, 10 aa, synthetic construct
ASGFYFPDYA

SEQ ID NO: 352 - protein, 19 aa, synthetic construct
MRGWAYGGSAQFAAFAVGK

SEQ ID NO: 353 - protein, 21 aa, synthetic construct
EQRNKDYRYGQEGFGYSYGMD

SEQ ID NO: 354 - protein, 8 aa, synthetic construct
ASHFIANY

SEQ ID NO: 355 - protein, 12 aa, synthetic construct
ESSTLQRGVPSR

SEQ ID NO: 356 - protein, 6 aa, synthetic construct
SHSPPV

SEQ ID NO: 357 - protein, 10 aa, synthetic construct
AFDFYFPDYA

SEQ ID NO: 358 - protein, 19 aa, synthetic construct
IRGWAYGQAAQYGKSASGR

SEQ ID NO: 359 - protein, 21 aa, synthetic construct
EQRGGDGRYSGDGFGYPYGMD

SEQ ID NO: 360 - protein, 12 aa, synthetic construct
QSWTLNRGIPSR

SEQ ID NO: 361 - protein, 6 aa, synthetic construct
SHSPPL

SEQ ID NO: 362 - protein, 19 aa, synthetic construct
IRGWAYGQSAQYGKSASGR

SEQ ID NO: 363 - protein, 21 aa, synthetic construct
EQRGANGRYGGDGFGYSYGMD

SEQ ID NO: 364 - protein, 12 aa, synthetic construct
ESSTLNRGVPSR

SEQ ID NO: 365 - protein, 132 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 366 - protein, 105 aa, synthetic construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 367 - protein, 132 aa, synthetic construct
QMQLQESGPGLVKPSETLSLTCSVSGASISDSYWSWIRRSPGKGLEWIGYVHKSGDTNYNPSLKSRVHLSLDTSKNQVSLSLTGVTAADSGKYYCARTLHGRRIYGIVAFNEWFTYFYMDVWGTGTQVTVSS

SEQ ID NO: 368 - protein, 105 aa, synthetic construct
SDISVAPGETARISCGEKSLGSRAVQWYQHRAGQAPSLIIYNNQDRPSGIPERFSGSPDFRPGTTATLTITSVEAGDEADYYCHIWDSRVPTKWVFGGGTTLTVL

SEQ ID NO: 369 - protein, 132 aa, synthetic construct; identical to SEQ ID NO: 367

SEQ ID NO: 370 - protein, 105 aa, synthetic construct
SDISVAPGETARISCGEKSLGSRAVQWYQHRAGQAPSLIIYNNQDRPSGIPERFSGSPDSRPGTTATLTITSVEAGDEADYYCHIWDSRVPTKWVFGGGTTLTVL

SEQ ID NO: 371 - protein, 132 aa, synthetic construct
QMQLQESGPGLVKPSETLSLTCSVSGASISDSYWSWIRQPPGKGLEWIGYVHKSGDTNYSPSLKSRVNLSLDTSKNQVSLSLSAATAADSGVYYCARTLHGRRIYGIVAFNEWFTYFYMDVWGNGTQVTVSS

SEQ ID NO: 372 - protein, 105 aa, synthetic construct
SDISVAPGETARISCGEKSLGSRAVQWYQQRAGQAPSLIIYNNQDRPSGIPERFSGSPDSGFGTTATLTITSVEAGDEADYYCHIWDSRVPTKWVFGGGTTLTVL

SEQ ID NO: 373 - protein, 132 aa, synthetic construct; identical to SEQ ID NO: 367

SEQ ID NO: 374 - protein, 105 aa, synthetic construct; identical to SEQ ID NO: 370

SEQ ID NO: 375 - protein, 132 aa, synthetic construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 376 - protein, 108 aa, synthetic construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 377 - protein, 132 aa, synthetic construct
QVQLQESGPGLVKPSETLSVTCSVSGDSMNNYYWTWIRQSPGKGLEWIGYISDRESATYNPSLNSRVTISRDTSKNQFSLKLNSVTPADTAVYYCARARRGQRIYGVVSFGEFFYYYSMDVWGKGTTVTVSS

SEQ ID NO: 378 - protein, 108 aa, synthetic construct
SPVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 379 - protein, 132 aa, synthetic construct
QVQLQESGPGLVRPSETLSVTCIVSGGSISNYYWTWIRQSPGKGLEWIGYISDRETTTYNPSLNSRAVISRDTSKNQLSLQLRSVTTADTAIYFCATARRGQRIYGVVSFGEFFYYYYMDVWGKGTAVTVSS

SEQ ID NO: 380 - protein, 107 aa, synthetic construct
SYVSPLSVALGETARISCGRQALGSRAVQWYQHKPGQAPILLIYNNQDRPSGIPERFSGTPDINFGTTATLTISGVEVGDEADYYCHMWDSRSGFSWSFGGATRLTV

SEQ ID NO: 381 - protein, 132 aa, synthetic construct
QVHLQESGPGLVTPSETLSLTCTVSNGSVSGRFWSWIRQSPGRGLEWIGYFSDTDRSEYNPSLRSRLTLSVDRSKNQLSLRLKSVTAADSATYYCARAQQGKRIYGIVSFGEFFYYYYMDAWGKGTPVTVSS

SEQ ID NO: 382 - protein, 107 aa, synthetic construct
SLNPLSLAPGATAKIPCGERSRGSRAVQWYQQKPGQAPTLIIYNNQDRPAGVSERFSGNPDVAIGVTATLTISRVEVGDEADYYCHYWDSRSPISWIFGGGTQLTVL

SEQ ID NO: 383 - protein, 132 aa, synthetic construct
QVHLQESGPGLVTPSETLSLTCTVSNGSVSGRFWSWIRQSPGRGLEWIGYFSDTDRSEYNPSLRSRLTLSVDRSKNQLSLKLKSVTAADSATYYCARAQQGKRIYGIVSFGELFYYYYMDAWGKGTPVTVSS

SEQ ID NO: 384 - protein, 107 aa, synthetic construct
SLNPLSLAPGATAKIPCGERSRGSRAVQWYQQKPGQAPTLIIYNNQDRPAGVSERFSGNPDVAIGVTATLTISRVEVGDEGDYYCHYWDSRSPISWIFAGGTQLTVL

SEQ ID NO: 385 - protein, 132 aa, synthetic construct
QVHLQESGPGLVKPSETLSLTCNVSGTLVRDNYWSWIRQPLGKQPEWIGYVHDSGDTNYNPSLKSRVHLSLDKSKNLVSLRLTGVTAADSAIYYCATTKHGRRIYGVVAFKEWFTYFYMDVWGKGTSVTVSS

SEQ ID NO: 386 - protein, 105 aa, synthetic construct
TFVSVAPGQTARITCGEESLGSRSVIWYQQRPGQAPSLIIYNNNDRPSGIPDRFSGSPGSTFGTTATLTITSVEAGDEADYYCHIWDSRRPTNWVFGEGTTLIVL

SEQ ID NO: 387 - protein, 132 aa, synthetic construct
QLHLQESGPGLVKPPETLSLTCSVSGASINDAYWSWIRQSPGKRPEWVGYVHHSGDTNYNPSLKRRVTFSLDTAKNEVSLKLVDLTAADSATYFCARALHGKRIYGIVALGELFTYFYMDVWGKGTAVTVSS

SEQ ID NO: 388 - protein, 105 aa, synthetic construct
SSMSVSPGETAKISCGKESIGSRAVQWYQQKPGQPPSLIIYNNQDRPAGVPERFSASPDFRPGTTATLTITNVDAEDEADYYCHIYDARGGTNWVFDRGTTLTVL

SEQ ID NO: 389 - protein, 135 aa, synthetic construct
QSQLQESGPRLVEASETLSLTCNVSGESTGACTYFWGWVRQAPGKGLEWIGSLSHCQSFWGSGWTFHNPSLKSRLTISLDTPKNQVFLKLTSLTAADTATYYCARFDGEVLVYNHWPKPAWVDLWGRGIPVTVSS

SEQ ID NO: 390 - protein, 105 aa, synthetic construct
QSALTQPPSASGSPGQSITISCNGTATNFVSWYQQFPDKAPKLIIFGVDKRPPGVPDRFSGSRSGTTASLTVSRLQTDDEAVYYCGSLVGNWDVIFGGGTTLTVL

SEQ ID NO: 391 - protein, 135 aa, synthetic construct
QPQLQESGPGLVEASETLSLTCTVSGDSTAACDYFWGWVRQPPGKGLEWIGGLSHCAGYYNTGWTYHNPSLKSRLTISLDTPKNQVFLKLNSVTAADTAIYYCARFDGEVLVYHDWPKPAWVDLWGRGTLVTVSS

SEQ ID NO: 392 - protein, 105 aa, synthetic construct
QSALTQPPSASGSPGQSISISCTGTSNRFVSWYQQHPGKAPKLVIYGVNKRPSGVPDRFSGSKSGNTASLTVSGLQTDDEAVYYCSSLVGNWDVIFGGGTKLTVL

SEQ ID NO: 393 - protein, 133 aa, synthetic construct [sequence omitted]

SEQ ID NO: 394 - protein, 107 aa, synthetic construct
EIVMTQSPDTLSVSPGETVTLSCRASQNINKNLAWYQYKPGQSPRLVIFETYSKIAAFPARFVASGSGTEFTLTINNMQSEDVAVYYCQQYEEWPRTFGQGTKVDIK

SEQ ID NO: 395 - protein, 134 aa, synthetic construct
QLQLQESGPGLVKPSETLSLTCTVSGGSMRGTDWGENDFHYGWIRQSSAKGLEWIGSIHWRGRTTHYKTSFRSRATLSIDTSNNRFSLTFSFVTAADTAVYYCARHKYHDIFRVVPVAGWFDPWGQGLLVTVSS

SEQ ID NO: 396 - protein, 107 aa, synthetic construct
EIVMTQSPPTLSVSPGETATLSCRASQNVKNNLAWYQLKPGQAPRLLIFDASSRAGGIPDRFSGSGYGTDFTLTVNSVQSEDFGDYFCQQYEEWPRTFGQGTKVDIK

SEQ ID NO: 397 - protein, 133 aa, synthetic construct
EVHLEESGPGLVRPSETLSLTCTASGGSIRGGEWGDSDYHWGWVRHSPEKGLEWIGSIHWRGTTHYNAPFRGRGRLSIDLSRNQFSLRLTSVTAEDTAVYYCVKHKYHDIVMVVPIAGWFDPWGQGLQVTVSS

SEQ ID NO: 398 - protein, 107 aa, synthetic construct
EIMMTQSPAILSVSPGDRATLSCRASQSVKNNLAWYQKRPGQAPRLLIFDTSSRASGIPARFSGGGSGTEFTLTVNSMQSEDFATYYCQQYEEWPRTFGQGTKVEIK

SEQ ID NO: 399 - protein, 135 aa, synthetic construct
QPQLQESGPGLVEASETLSLTCTVSGDSTAACDYFWGWVRQPPGKGLEWIGSLSHCAGYYNSGWTYHNPSLKSRLTISLDTPKNQVFLKLNSVTAADTAIYYCARFGGDVLVYHDWPKPAWVDLWGRGVLVTVSS

SEQ ID NO: 400 - protein, 105 aa, synthetic construct
QSALTQPPSASGSPGQSITISCTGNINNFVSWYQQHPGKAPKLVIYGVNKRPSGVPDRFSGSKSGNAASLTVSGLQTDDEAVYYCGSLAGNWDVVFGGGTKLTVL

SEQ ID NO: 401 - protein, 135 aa, synthetic construct
QPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSS

SEQ ID NO: 402 - protein, 105 aa, synthetic construct
QSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVL

SEQ ID NO: 403 - protein, 135 aa, synthetic construct
QPQLQESGPGLVEASETLSLTCTVSGDSTAGCDYFWGWVRQPPGKGLEWIGGLSHCAGYYNTGWTYHNPSLKSRLTISLDTPKNQVFLKLNSVTAADTAIYYCARFDGEVLVYNDWPKPAWVDLWGRGTLVTVSS

SEQ ID NO: 404 - protein, 105 aa, synthetic construct
QSALTQPPSASGSPGQSITISCTGTSNNFVSWYQQHPAKAPKLVIYGVNKRPSGVPDRFSGSKSGNTASLTVSGLQTDDEAVYYCGSLVGNWDVIFGGGTKLTVL

SEQ ID NO: 405 - protein, 129 aa, synthetic construct
QVQLQESGPGLVKPAETLSLTCSVSGESINTGHYYWGWVRQVPGKGLEWIGHIHYTTAVLHNPSLKSRLTIKIYTLRNQITLRLSNVTAADTAVYHCVRSGGDILYYYEWQKPHWFSPWGPGIHVTVSS

SEQ ID NO: 406 - protein, 110 aa, synthetic construct
QSALTQPPSASGSLGQSVTISCNGTSSDIGGWNFVSWYQQFPGRAPRLIIFEVNKRPSGVPGRFSGSKSGNSASLTVSGLQSDDEGQYFCSSLFGRWDVVFGGGTKLTVL

SEQ ID NO: 407 - protein, 130 aa, synthetic construct
QVQLRESGPGLVKPSETLSLSCTVSQDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSS

SEQ ID NO: 408 - protein, 110 aa, synthetic construct
WASSELTQPPSVSVSPGQTARITCSGAPLTSRFTYWYRQKPGQAPVLIISRSSQRSSGWSGRFSASWSGTTVTLTIRGVQADDEADYYCQSSDTSDSYKMFGGGTKLTVL

SEQ ID NO: 409 - protein, 130 aa, synthetic construct
QVQLRESGPGLVKPSETLSLSCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSS

SEQ ID NO: 410 - protein, 108 aa, synthetic construct
SSELTQPPSVSVSPGQTARITCSGAPLTSRFTYWYRQKPGQAPVLIISRSSQRSSGWSGRFSASWSGTTVTLTIRGVQADDEADYYCQSSDTSDSYKMFGGGTKLTVL

SEQ ID NO: 411 - protein, 130 aa, synthetic construct
EVQLRESGPRLVKPSETLSLSCDVFGDSRPSDHSWTWVRQPPGKALEWIGDVHYNGDNTYNPSLRGRVKIDVDRSTHRFSLTLKSLTAADTGIYFCARNVIRVFGVISLGEWFHYGMDVWGPGTAVIVSS

SEQ ID NO: 412 - protein, 108 aa, synthetic construct
SSELTQAPSVSVSPGQTATIACSGPPLASRYTYWYRQKPGQAPVLIIFRDRQFPSGVSGRFSASKSGTTATLTIRDVQVEDEGDYYCQSSDTSDSYKMFGGGTTLTVL

SEQ ID NO: 413 - protein, 130 aa, synthetic construct
EVQLRESGPGLVKPSETLSLSCDVFGDSRPSDHSWTWVRQPPGKALEWIGDVHYNGDTTYNPSLRGRVKIDVDRSTHRFSLTLNSLTAADTGIYFCARNVIRVFGVISLGEWFHYGMDVWGQGTAVTVSS

SEQ ID NO: 414 - protein, 108 aa, synthetic construct; identical to SEQ ID NO: 412

SEQ ID NO: 415 - protein, 130 aa, synthetic construct
QVQLRESGPGLVKPSETLSLTCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSS

SEQ ID NO: 416 - protein, 108 aa, synthetic construct
SSELTQPPSVSVSPGQTAKITCSGAALTSRFTYWYRQKPGQAPVLIISRTSQRSSGWSGRFSASWSGTTVTLTIRGVQADDEGDYYCQSSDTSDSYKMFGGGTKLTVL

SEQ ID NO: 417 - protein, 130 aa, synthetic construct
EVQLRESGPGLVKPSGNMALTCTISGDSRPSDHSWTWVRQSPGKALEWIGDIHYGGDITYNPSLRSRVKLEVDTSTNRFFLKMTSLTVADTGIYFCARNVIRVFGVIALGEWFHYGMDVWGQGTAITVSP

SEQ ID NO: 418 - protein, 108 aa, synthetic construct
SSELTQTPSVTVSPGETARIACSGPPLASRYCYWYRQKPGQAPVLIIFRDRQFSSGMSGRFASSHSGTTVTLTIRDVRVEDEADYYCQSSDINDSYKMFGGGTKVTVL

SEQ ID NO: 419 - protein, 130 aa, synthetic construct
EVQLRESGPGLVKPSGNMALTCTISGDSRPSDHSWTWVRQSPGKTLEWIGDIHYGGDITYNPSLRSRVKLEVDTSSNRFFLKMTSLTVADTGIYFCARNVIRVFGVIALGEWFHYGMDVWGQGTAITVSP

SEQ ID NO: 420 - protein, 108 aa, synthetic construct
SSELTQTASVTVSPGETARIACSGPPLASRYCYWYRQKPGQAPVLIIFRDRQFSSGISGRFSSSQSGTTVTLTIRDVRVEDEADYYCQSSDTSDSFKMFGGGTKLTVL

SEQ ID NO: 421 - protein, 130 aa, synthetic construct
QVQLRESGPGLVKPSGNMALTCTISGDSRPSDHSWTWVRQSPGKALEWIGDIHYGGDITYNPSLRSRVELEVDRSTNRFFLKMTSLSVADTGMYFCARNVIRVFGVIALGEWFHYGMDVWGQGTAITVSP

SEQ ID NO: 422 - protein, 108 aa, synthetic construct
SSELTQAPSVTVSPGDTARIACSGPPLATRYCYWYRQKSGQAPVLIIFRDRQFSSGVSGRFSSSQSGSTVTLTIRDVRVEDEADYYCQSSDTSDSYKMFGGGTKLTVL

SEQ ID NO: 423 - protein, 128 aa, synthetic construct
QVQLRESGPGLVKPSETLSLSCDVFGDSRPSDHSWTWVRQPPGKALEWIGDIHYNGDKTYNPSLRGRVKIDVDRSTHRFSLTLNSLTAADTGMYFCARNVIRVFGVISLGEWFHYGMDVWGPGTAVTV

SEQ ID NO: 424 - protein, 108 aa, synthetic construct
SSELTQAPSVSVSPGQTARIACSGPPLASRYTYWYRQKPGQAPVLIIFRDRQFPSGVSGRFSASKSGTTGTLTIRDVQAEDEGDYYCQSSDTSDSYKMFGGGTTLTVL

SEQ ID NO: 425 - protein, 130 aa, synthetic construct
QVQLRESGPGLVKPSGNMALTCTISGDSRPSDHSWTWVRQSPGKALEWIGDIHYGGDITYNPSLRSRVKLEVDTSSNRFFLKMTSLTVADTGIYFCARNVIRVFGVIALGEWFHYGMDVWGQGTAITVSP

SEQ ID NO: 426 - protein, 108 aa, synthetic construct
SSELTQAPSVTLSPGETARIACSGPPLASRYCYWYRQKPGQAPVLIIFRDRQFSSGISGRFSSSQSGTTVTLTIRDVRVEDEADYYCQSSDNSDSFKMFGGGTKLTVL

SEQ ID NO: 427 - protein, 133 aa, synthetic construct
AEQLVESGGGLVPPGRSLRLSCSASGFYFPDYAMAWVRQAPGQGLQWVGFMRGWAYGGSAQFAAFAVGKFAISRDDGRNVVYLDVKNPTFEDTGVYFCAREQRNKDYRYGQEGFGYSYGMDVWGRGTTVVVST

SEQ ID NO: 428 - protein, 107 aa, synthetic construct
DIHMTQSPVSLSASVGDRVTITCRASHFIANYVNWYQQKPGKAPTLLIFESSTLQRGVPSRFSAYGDGTEFTLSINTLQPEDFASYICQQSHSPPVTFGAGTRVDQK

SEQ ID NO: 429 - protein, 133 aa, synthetic construct
EERLVESGGGLVPPGRSLRLSCSAFDFYFPDYAMAWVRQAPGKGLEWIGFIRGWAYGQAAQYGKSASGRMTISRDDSRRVVYLDIKSPIEEDTGAYFCAREQRGGDGRYSGDGFGYPYGMDVWGRGTMVTVSA

SEQ ID NO: 430 - protein, 107 aa, synthetic construct
DILMTQSPVSLSASIGERITITCRASHFIANYVNWYQQRPGKAPKLLIFQSWTLNRGIPSRFSGYGDGTEFTLSISALQSEDFGTYICQQSHSPPLSFGGGTRVDQT

SEQ ID NO: 431 - protein, 133 aa, synthetic construct
EERLVESGGGLVPPGRSLRLSCSAFDFYFPDYAMAWVRQAPGRALEWIGFIRGWAYGQSAQYGKSASGRMTISRDDSRRVVYLDIKSPTHEDTGVYFCAREQRGANGRYGGDGFGYSYGMDVWGRGTMVSVSA

SEQ ID NO: 432 - protein, 107 aa, synthetic construct
DIQMTQSPFTLSASVGERVTITCRASHFIANYVNWYQQRPGRAPKLLIFESSTLNRGVPSRFSGSGDGTEFTLSISALQSEDFATYICQQSHSPPVSFGGGTRVDQT

SEQ ID NO: 433 - protein, 461 aa, synthetic construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 434 - protein, 214 aa, synthetic construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 435 - protein, 511 aa, Human immunodeficiency virus 1; identical to SEQ ID NO: 4

SEQ ID NO: 436 - protein, 856 aa, Human immunodeficiency virus 1; identical to SEQ ID NO: 3

SEQ ID NO: 437 - protein, 511 aa, Human immunodeficiency virus 1; identical to SEQ ID NO: 4

SEQ ID NO: 438 - protein, 475 aa, Human immunodeficiency virus 1; identical to SEQ ID NO: 5

SEQ ID NO: 439 - protein, 481 aa, synthetic construct; identical to SEQ ID NO: 6

SEQ ID NO: 440-441 - skipped (2 entries)

SEQ ID NO: 442 - protein, 5 aa, synthetic construct
DYFIH

SEQ ID NO: 443 - protein, 17 aa, synthetic construct
WINPKTGQPNNPRQFQG

SEQ ID NO: 444 - protein, 10 aa, synthetic construct
QRSDYWDFDV

SEQ ID NO: 445 - protein, 7 aa, synthetic construct
QANGYLN

SEQ ID NO: 446 - protein, 7 aa, synthetic construct
DGSKLER

SEQ ID NO: 447 - protein, 5 aa, synthetic construct
QVYEF

SEQ ID NO: 448 - protein, 5 aa, synthetic construct
DHFIH

SEQ ID NO: 449 - protein, 10 aa, synthetic construct
QRSDFWDFDV

SEQ ID NO: 450 - protein, 5 aa, synthetic construct
NCPIN

SEQ ID NO: 451 - protein, 17 aa, synthetic construct
WMKPRGGAVSYARQLQG

SEQ ID NO: 452 - protein, 16 aa, synthetic construct
GKYCTARDYYNWDFEH

SEQ ID NO: 453 - protein, 9 aa, synthetic construct
RTSQYGSLA

SEQ ID NO: 454 - protein, 7 aa, synthetic construct
SGSTRAA

SEQ ID NO: 455 - protein, 5 aa, synthetic construct
QQYEF

SEQ ID NO: 456 - protein, 17 aa, synthetic construct
WMKPRHGAVSYARQLQG

SEQ ID NO: 457 - protein, 5 aa, synthetic construct
DCTLN

SEQ ID NO: 458 - protein, 16 aa, synthetic construct
WLKPRGGAVNYARPLQ

SEQ ID NO: 459 - protein, 12 aa, synthetic construct
GKNCDYNWDFEH

SEQ ID NO: 460 - protein, 5 aa, synthetic construct
AHILF

SEQ ID NO: 461 - protein, 17 aa, synthetic construct
WIKPQYGAVNFGGGFRD

SEQ ID NO: 462 - protein, 13 aa, synthetic construct
DRSYGDSSWALDA

SEQ ID NO: 463 - protein, 11 aa, synthetic construct
QTSQGVGSDLH

SEQ ID NO: 464 - protein, 7 aa, synthetic construct
HTSSVED

SEQ ID NO: 465 - protein, 5 aa, synthetic construct
QVLQF

SEQ ID NO: 466 - protein, 11 aa, synthetic construct
DDDTFTKYWTH

SEQ ID NO: 467 - protein, 17 aa, synthetic construct
VISPHFARPIYSYKFRD

SEQ ID NO: 468 - protein, 16 aa, synthetic construct
DPFGDRAPHYNYHMDV

SEQ ID NO: 469 - protein, 12 aa, synthetic construct
RASQGLDSSHLA

SEQ ID NO: 470 - protein, 7 aa, synthetic construct
GTSNRAR

SEQ ID NO: 471 - protein, 9 aa, synthetic construct
QRYGGTPIT

SEQ ID NO: 472 - protein, 6 aa, synthetic construct
RTELIH

SEQ ID NO: 473 - protein, 17 aa, synthetic construct
WVKTVTGAVNFGSPDFR

SEQ ID NO: 474 - protein, 14 aa, synthetic construct
QKFYTGGQGWYFDL

SEQ ID NO: 475 - protein, 9 aa, synthetic construct
TAASYGHMT

SEQ ID NO: 476 - protein, 7 aa, synthetic construct
ATSKRAS

SEQ ID NO: 477 - protein, 5 aa, synthetic construct
QQLEF

SEQ ID NO: 478 - protein, 7 aa, synthetic construct
GYNIRDY

SEQ ID NO: 479 - protein, 4 aa, synthetic construct
PKTG

SEQ ID NO: 480 - protein, 8 aa, synthetic construct
RSDYWDFD

SEQ ID NO: 481-483 - skipped (3 entries)

SEQ ID NO: 484 - protein, 7 aa, synthetic construct
GYKISDH

SEQ ID NO: 485 - protein, 8 aa, synthetic construct
RSDFWDFD

SEQ ID NO: 486 - protein, 7 aa, synthetic construct
GYEFINC

SEQ ID NO: 487 - protein, 4 aa, synthetic construct
PRGG

SEQ ID NO: 488 - protein, 14 aa, synthetic construct
KYCTARDYYNWDFE

SEQ ID NO: 489 - protein, 5 aa, synthetic construct
SQYGS

SEQ ID NO: 490 - skipped

SEQ ID NO: 491 - protein, 4 aa, synthetic construct
PRHG

SEQ ID NO: 492 - protein, 7 aa, synthetic construct
GYEFIDC

SEQ ID NO: 493 - protein, 10 aa, synthetic construct
KNCDYNWDFE

SEQ ID NO: 494 - protein, 7 aa, synthetic construct
GYTFTAH

SEQ ID NO: 495 - protein, 4 aa, synthetic construct
PQYG

SEQ ID NO: 496 - protein, 11 aa, synthetic construct
RSYGDSSWALD

SEQ ID NO: 497 - protein, 7 aa, synthetic construct
SQGVGSD

SEQ ID NO: 498-499 - skipped (2 entries)

SEQ ID NO: 500 - protein, 13 aa, synthetic construct
DDPYTDDDTFTKY

SEQ ID NO: 501 - protein, 4 aa, synthetic construct
PHFA

SEQ ID NO: 502 - protein, 14 aa, synthetic construct
PFGDRAPHYNYHMD

SEQ ID NO: 503 - protein, 8 aa, synthetic construct
SQGLDSSH

SEQ ID NO: 504 - skipped

SEQ ID NO: 505 - protein, 6 aa, synthetic construct
YGGTPI

SEQ ID NO: 506 - protein, 8 aa, synthetic construct
EDIFERTE

SEQ ID NO: 507 - protein, 4 aa, synthetic construct
TVTG

SEQ ID NO: 508 - protein, 12 aa, synthetic construct
KFYTGGQGWYFD

SEQ ID NO: 509 - protein, 5 aa, synthetic construct
ASYGH

SEQ ID NO: 510-511 - skipped (2 entries)

SEQ ID NO: 512 - protein, 8 aa, synthetic construct
GYNIRDYF

SEQ ID NO: 513 - protein, 8 aa, synthetic construct
INPKTGQP

SEQ ID NO: 514 - protein, 12 aa, synthetic construct
ARQRSDYWDFDV

SEQ ID NO: 515 - protein, 8 aa, synthetic construct
GYKISDHF

SEQ ID NO: 516 - protein, 12 aa, synthetic construct
ARQRSDFWDFDV

SEQ ID NO: 517 - protein, 8 aa, synthetic construct
GYEFINCP

SEQ ID NO: 518 - protein, 8 aa, synthetic construct
MKPRGGAV

SEQ ID NO: 519 - protein, 18 aa, synthetic construct
TRGKYCTARDYYNWDFEH

SEQ ID NO: 520 - protein, 4 aa, synthetic construct
QYGS

SEQ ID NO: 521 - protein, 8 aa, synthetic construct
MKPRHGAV

SEQ ID NO: 522 - protein, 8 aa, synthetic construct
GYEFIDCT

SEQ ID NO: 523 - protein, 8 aa, synthetic construct
LKPRGGAV

SEQ ID NO: 524 - protein, 14 aa, synthetic construct
TRGKNCDYNWDFEH

SEQ ID NO: 525 - protein, 8 aa, synthetic construct
GYTFTAHI

SEQ ID NO: 526 - protein, 8 aa, synthetic construct
IKPQYGAV

SEQ ID NO: 527 - protein, 15 aa, synthetic construct
ARDRSYGDSSWALDA

SEQ ID NO: 528 - protein, 6 aa, synthetic construct
QGVGSD

SEQ ID NO: 529 - protein, 14 aa, synthetic construct
DDPYTDDDTFTKYW

SEQ ID NO: 530 - protein, 8 aa, synthetic construct
ISPHFARP

SEQ ID NO: 531 - protein, 18 aa, synthetic construct
ARDPFGDRAPHYNYHMDV

SEQ ID NO: 532 - protein, 7 aa, synthetic construct
QGLDSSH

SEQ ID NO: 533 - protein, 9 aa, synthetic construct
EDIFERTEL

SEQ ID NO: 534 - protein, 8 aa, synthetic construct
VKTVTGAV

SEQ ID NO: 535 - protein, 16 aa, synthetic construct
ARQKFYTGGQGWYFDL

SEQ ID NO: 536 - protein, 4 aa, synthetic construct
SYGH

SEQ ID NO: 537 - skipped

SEQ ID NO: 538 - protein, 10 aa, synthetic construct
ASGYNIRDYF

SEQ ID NO: 539 - protein, 17 aa, synthetic construct
INPKTGQPNNPRQFQGR

SEQ ID NO: 540 - protein, 9 aa, synthetic construct
QRSDYWDFD

SEQ ID NO: 541 - protein, 4 aa, synthetic construct
ANGY

SEQ ID NO: 542 - protein, 13 aa, synthetic construct
DGSKLERGVPSRF

SEQ ID NO: 543 - protein, 10 aa, synthetic construct
ASGYKISDHF

SEQ ID NO: 544 - protein, 9 aa, synthetic construct
QRSDFWDFD

SEQ ID NO: 545 - protein, 12 aa, synthetic construct
DGSKLERGVPAR

SEQ ID NO: 546 - protein, 10 aa, synthetic construct
ASGYEFINCP

SEQ ID NO: 547 - protein, 17 aa, synthetic construct
MKPRGGAVSYARQLQGR

SEQ ID NO: 548 - protein, 15 aa, synthetic construct
GKYCTARDYYNWDFE

SEQ ID NO: 549 - protein, 6 aa, synthetic construct
TSQYGS

SEQ ID NO: 550 - protein, 12 aa, synthetic construct
SGSTRAAGIPDR

SEQ ID NO: 551 - protein, 17 aa, synthetic construct
MKPRHGAVSYARQLQGR

SEQ ID NO: 552 - protein, 10 aa, synthetic construct
ASGYEFIDCT

SEQ ID NO: 553 - protein, 17 aa, synthetic construct
LKPRGGAVNYARPLQGR

SEQ ID NO: 554 - protein, 11 aa, synthetic construct
GKNCDYNWDFE

SEQ ID NO: 555 - protein, 10 aa, synthetic construct
TSGYTFTAHI

SEQ ID NO: 556 - protein, 17 aa, synthetic construct
IKPQYGAVNFGGGFRDR

SEQ ID NO: 557 - protein, 12 aa, synthetic construct
DRSYGDSSWALD

SEQ ID NO: 558 - protein, 8 aa, synthetic construct
TSQGVGSD

SEQ ID NO: 559 - protein, 12 aa, synthetic construct
HTSSVEDGVPSR

SEQ ID NO: 560 - protein, 16 aa, synthetic construct
ADDDPYTDDDTFTKYW

SEQ ID NO: 561 - protein, 17 aa, synthetic construct
ISPHFARPIYSYKFRDR

SEQ ID NO: 562 - protein, 15 aa, synthetic construct
DPFGDRAPHYNYHMD

SEQ ID NO: 563 - protein, 9 aa, synthetic construct
ASQGLDSSH

SEQ ID NO: 564 - protein, 12 aa, synthetic construct
GTSNRARGTPDR

SEQ ID NO: 565 - protein, 11 aa, synthetic construct
TSEDIFERTEL

SEQ ID NO: 566 - protein, 17 aa, synthetic construct
VKTVTGAVNFGSPDFRQ

SEQ ID NO: 567 - protein, 13 aa, synthetic construct
QKFYTGGQGWYFD

SEQ ID NO: 568 - protein, 6 aa, synthetic construct
AASYGH

SEQ ID NO: 569 - protein, 12 aa, synthetic construct
ATSKRASGIPDR

SEQ ID NO: 570 - skipped

SEQ ID NO: 571 - protein, 123 aa, synthetic construct
QVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSS

SEQ ID NO: 572 - protein, 99 aa, synthetic construct
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLK

SEQ ID NO: 573 - protein, 123 aa, synthetic construct
QVQLLQSGAAVTKPGASVRVSCEASGYNIRDYFIHWWRQAPGQGLQWVGWINPKTGQPNNPRQFQGRVSLTRHASFDFDTFSFYMDLKALRSDDTAVYFCARQRSDYWDFDVWGSGTQVTVSS

SEQ ID NO: 574 - protein, 99 aa, synthetic construct
DIQMTQSPSSLSASVGDTATITCQANGYLNWYQQRRGKAPKLLIYDGSKLERGVPSRFSGRRWGQEYNLTINNLQPEDIATYFCQVYEFVVPGTRLDLK

SEQ ID NO: 575 - protein, 123 aa, synthetic construct
QVHLSQSGAAVTKPGASVRVSCEASGYKISDHFIHWWRQAPGQGLQWVGWINPKTGQPNNPRQFQGRVSLTRQASWDFDTYSFYMDLKAVRSDDTAIYFCARQRSDFWDFDVWGSGTQVTVSS

SEQ ID NO: 576 - protein, 99 aa, synthetic construct
DIQMTQSPSSLSARVGDTVTITCQANGYLNWYQQRRGKAPKLLIYDGSKLERGVPARFSGRRWGQEYNLTINNLQPEDVATYFCQVYEFIVPGTRLDLK

SEQ ID NO: 577 - protein, 125 aa, synthetic construct
QVRLSQSGGQMKKPGDSMRISCRASGYEFINCPINWIRLAPGKRPEWMGWMKPRGGAVSYARQLQGRVTMTRDMYSETAFLELRSLTSDDTAVYFCTRGKYCTARDYYNWDFEHWGQGTPVTVSS

SEQ ID NO: 578 - protein, 103 aa, synthetic construct
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 579 - protein, 125 aa, synthetic construct
QVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSS

SEQ ID NO: 580 - protein, 101 aa, synthetic construct
SLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 581 - protein, 121 aa, synthetic construct
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSS

SEQ ID NO: 582 - protein, 103 aa, synthetic construct; identical to SEQ ID NO: 578

SEQ ID NO: 583 - protein, 122 aa, synthetic construct [sequence omitted]

SEQ ID NO: 584 - protein, 103 aa, synthetic construct [sequence omitted]

SEQ ID NO: 585 - protein, 131 aa, synthetic construct
QGRLFQSGAEVKRPGASVRISCRADDDPYTDDDTFTKYWTHWIRQAPGQRPEWLGVISPHFARPIYSYKFRDRLTLTRDSSLTAVYLELKGLQPDDSGIYFCARDPFGDRAPHYNYHMDVWGGGTAVIVSS

SEQ ID NO: 586 - protein, 112 aa, synthetic construct
EVVLTQSPAILSVSPGDRVILSCRASQGLDSSHLAWYRFKRGQIPTLVIFGTSNRARGTPDRFSGSGSGADFTLTISRVEPEDFATYYCQRYGGTPITFGGGTTLDKKRTVA

SEQ ID NO: 587 - protein, 125 aa, synthetic construct
QVQLVQSGSGVKKPGASVRVSCWTSEDIFERTELIHWVRQAPGQGLEWIGWVKTVTGAVNFGSPDFRQRVSLTRDRDLFTAHMDIRGLTQGDTATYFCARQKFYTGGQGWYFDLWGRGTLIVVSS

SEQ ID NO: 588 - protein, 105 aa, synthetic construct
EIVLTQSPGTLSLSPGETASLSCTAASYGHMTWYQKKPGQPPKLLIFATSKRASGIPDRFSGSQFGKQYTLTITRMEPEDFARYYCQQLEFFGQGTRLEIRRTVA

SEQ ID NO: 589 - protein, 452 aa, synthetic construct
QVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 590 - protein, 206 aa, synthetic construct
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0083984 A1
(43) Pub. Date: Mar. 14, 2024

(54) DOSING AND SCHEDULING REGIMEN FOR BROADLY NEUTRALIZING ANTIBODIES

(71) Applicant: Gilead Sciences, Inc., Foster City, CA (US)

(72) Inventors: Yanan Zheng, Cupertino, CA (US); Tomas Cihlar, Burlingame, CA (US); Diana M. Brainard, Lexington, MA (US)

(21) Appl. No.: 18/448,721

(22) Filed: Aug. 11, 2023

Related U.S. Application Data
(60) Provisional application No. 63/514,711, filed on Jul. 20, 2023.
(60) Provisional application No. 63/373,597, filed on Aug. 26, 2022.

(51) Int. Cl. C07K 16/10; A61K 31/416; A61K 45/06; A61P 31/18

(52) U.S. Cl. CPC C07K 16/1063; A61K 31/416; A61K 45/06; A61P 31/18; A61K 2039/507

(57) ABSTRACT

Provided are methods for administering long-acting anti-HIV broadly neutralizing antibodies twice annually, e.g., Q6M, Q24W, Q25W or Q26W.

### CROSS-REFERENCE TO RELATED APPLICATIONS

This application claims the benefit under 35 U.S.C. § 119(e) of U.S. Provisional Application No. 63/373,597, filed on Aug. 26, 2022 and U.S. Provisional Application No. 63/514,711, filed on Jul. 20, 2023, which are hereby incorporated herein by reference in their entireties for all purposes.

### SEQUENCE LISTING

The instant application contains a Sequence Listing which has been submitted electronically in .XML format and is hereby incorporated by reference in its entirety. Said .XML copy, created on Jul. 20, 2023, is named 1445-WO-PCT_sequencelisting.XML and is 512,134 bytes in size.

### BACKGROUND

Human immunodeficiency virus type 1 (HIV-1) infection causes a serious life-threatening disease and remains one of the leading causes of morbidity and mortality worldwide. In the United States (US), there are approximately 1 million people with HIV (PWH) infection, and globally there are over 38 million (UNAIDS. Fact Sheet—Global HIV Statistics 2021). Advances in antiretroviral (ARV) therapy (ART) for HIV have led to significant improvements in morbidity and mortality by suppressing viral replication, preserving immunologic function, and averting disease progression to AIDS. However, current therapeutic strategies have been unable to eliminate the virus and cure HIV-1 infection.

While current combination ART for the treatment of HIV-1 infection is efficacious and well tolerated, these agents need to be taken every day and require near-perfect adherence to minimize the emergence of drug-resistant variants. As a result, “treatment fatigue” can occur, defined as “decreased desire and motivation to maintain vigilance in adhering to a treatment regimen” among patients prescribed chronic or lifelong treatment (Claborn, et al., Psychol Health Med (2015) 20(3):255-65), which can lead to nonadherence and treatment failure. As such, there remains a significant medical need for ARVs that can be administered less frequently (i.e, long-acting drug products), thereby providing an alternative treatment option for HIV-1 infected individuals.

Lenacapavir is a novel, first-in-class, multistage, selective inhibitor of HIV-1 capsid function targeted for the treatment of HIV-1 infection. Lenacapavir has potent antiviral activity with no overlapping resistance with any approved products. It has a low human clearance and is being developed as a long-acting ARV for treatment and for the prevention of HIV-1. Lenacapavir has the potential to meet the high unmet medical need in PWH who could benefit from long-acting treatment or a novel mechanism of action.

Monoclonal antibodies (mAbs) with neutralizing activity against HIV-1 envelope glycoproteins of increasing potency and breadth have been identified (Burton and Mascola, Nat Immunol (2015) 16(6):571-6) and the parenteral administration of broadly neutralizing mAbs produce significant reductions in plasma viremia in untreated PWH and have maintained virologic suppression in virologically suppressed PWH who have received broadly neutralizing antibodies (bNAbs) prior to undergoing analytic treatment interruption (Caskey, et al., Nature (2015) 522 (7557):487-91; Caskey, et al., Nat Med (2017) 23 (2):185-91; Mendoza, et al., Nature (2018) 561:479-84). Antibodies can be long acting and have the potential to mitigate the challenges or lifelong adherence to daily therapy. Antibodies also engage the immune system which may contribute to a beneficial HIV specific immune response (Niessl, et al., Nat Med (2020) 26 (2):222-7), including the potential clearance of latently infected cells (Gaebler, et al., Nature (2022) 606(7913):368-374), that is not achieved by ARV drugs. As biologics, bNAbs may spare patients from adverse effects associated with chronic ARV therapy. HIV-1, however, is a diverse virus whose variants have varying levels of sensitivity for any bNAb. Therefore, bNAbs identified to date have incomplete breadth when measured for their ability to neutralize a diversity of HIV-1 isolates (Nishimura, et al., Nature (2017) 543(7646):559-63). 3BNC117 and 10-1074 are two of the most potent bNAbs that have been identified and clinically tested (Mouquet, et al., Proc Natl Acad Sci USA (2012) 109 (47):E3268-77; Scheid, et al., Science (2011) 333(6049):1633-7). However, viral resistance to bNAbs can occur after antibody titer wanes (Bar-On, et al., Nat Med (2018) 24:1701-7).

### SUMMARY

In one aspect, provided are methods of treating or preventing HIV in a human subject in need thereof. In some embodiments, the methods comprise: (a) Co-administering at a first time point (i) an effective amount of a first antibody that competes with or comprises VH and VL regions that bind to an epitope of gp120 within the third variable loop (V3) and/or high mannose patch comprising a N332 oligomannose glycan and (ii) an effective amount of a second antibody that competes with or comprises VH and VL regions that bind to an epitope of gp120 comprising the CD4 binding site (CD4bs), wherein the first antibody and the second antibody both comprise Fe amino acid substitutions to extend serum half-life; and (b) Co-administering at a second time point at least about 24 weeks, e.g., at least about 25 weeks, e.g., at least about 26 weeks, after the first time point an effective amount of the first antibody and an effective amount of the second antibody. In some embodiments, the first antibody and the second antibody comprise an Fc region comprising the following amino acids at the indicated positions (EU index numbering): (i) Tyrosine at position 252, threonine at position 254 and glutamic acid at position 256 (YTE); (ii) Leucine at position 428 and serine at position 434 (LS); (iii) Lysine at position 433 and phenylalanine at position 434; (iv) Glutamine at position 250 and leucine at position 428 (QL); (v) Glutamine at position 307, valine at position 311 and valine at position 378 (DF215); (vi) Aspartic acid at position 256, aspartic acid at position 286, arginine at position 307, valine at position 311 and valine at position 378 (DF228); or (vii) aspartic acid at position 309, histidine at position 311 and serine at position 434 (DHS). In some embodiments, the first antibody competes with or comprises VH and VL regions of an antibody selected from GS-2872 (a.k.a., zinlirvimab), 10-1074, 10-1074-J, GS-9722, GS-9721, PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03; and the second antibody competes with or comprises VH and VL regions of an antibody selected from GS-5423, 3BNC117, GS-9723, 3BNC60, b12, F105, VRC01, VRC07, VRC07-523, VRC03, VRC06, VRC06b01 VRC08, VRC0801, NIH45-46, PGV04 (VRC-PG04); CH103, 44-VRC13.01, 1NC9, 12A12, N6, 1-18, N49-P7, NC-Cow1, IOMA, CH235 and CH235.12, N49P6, N49P7, N49P11, N49P9 and N60P25. In some embodiments, the first antibody competes with or comprises VH and VL regions of 10-1074 and the second antibody competes with or comprises VH and VL regions of 3BNC117. In some embodiments, the first antibody comprises 10-1074-LS (a.k.a., zinlirvimab; GS-2872) and the second antibody comprises 3BNC117-LS (a.k.a., teropavimab; GS-5423). In some embodiments, the first antibody and the second antibody are co-administered every 6 months (Q6M). In some embodiments, the first antibody and the second antibody are co-administered every 24 weeks (Q24W). In some embodiments, the first antibody and the second antibody are co-administered every 25 weeks (Q25W). In some embodiments, the first antibody and the second antibody are co-administered every 26 weeks (Q26W). In some embodiments, the first antibody and the second antibody are independently administered intravenously at a dose in the range of from about 500 mg to about 3000 mg, e.g., from about 550 mg to about 2900 mg, e.g., from about 600 mg to about 2800 mg, e.g., from about 650 mg to about 2700 mg, e.g., from about 700 mg to about 2600 mg, e.g., from about 850 mg to about 2550 mg. In some embodiments, the first antibody is administered intravenously at a dose of 2550 mg and the second antibody is administered intravenously at a dose of 2550 mg. In some embodiments, the first antibody is administered intravenously at a dose of 850 mg and the second antibody is administered intravenously at a dose of 1275 mg. In some embodiments, the first antibody is administered intravenously at a dose of 850 mg and the second antibody is administered intravenously at a dose of 1700 mg. In some embodiments, the first antibody is administered intravenously at a dose of 850 mg and the second antibody is administered intravenously at a dose of 2550 mg. In some embodiments, the methods further comprise co-administering one or more long-acting HIV drugs. In some embodiments, the one or more long-acting HIV drugs are selected from a long-acting capsid inhibitor, a long-acting integrase strand transfer inhibitor (INSTI), a long-acting non-nucleoside reverse transcriptase inhibitor (NNRTI), a long-acting nucleoside reverse transcriptase inhibitors (NRTI), and a long-acting protease inhibitor (PI). In some embodiments, the one or more long-acting HIV drugs comprises a long-acting capsid inhibitor. In some embodiments, the long-acting capsid inhibitor is selected from lenacapavir, VH4004280 and VH4011499. In some embodiments, the long-acting capsid inhibitor comprises lenacapavir. In some embodiments, the lenacapavir is administered at a dose in the range of 300 mg to 1000 mg. In some embodiments, the lenacapavir is administered orally or subcutaneously. In some embodiments, the long-acting INSTI is selected from bictegravir, raltegravir, elvitegravir, dolutegravir, cabotegravir, GS-1720, GS-6212, GS-1219, GS-3242 and VH4524184. In some embodiments, the long-acting NNRTI is selected from rilpivirine, elsulfavirine, doravirine and GS-5894. In some embodiments, the long-acting NRTI is selected from islatravir and prodrugs thereof, tenofovir alafenamide (TAF) and prodrugs of tenofovir, rovafovir etalafenamide and GS-1614. In some embodiments, the long-acting protease inhibitor is selected from atazanavir, ritonavir, darunavir, GS-1156 and prodrugs of GS-1156, and combinations thereof. In some embodiments, the methods further comprise determining the sensitivity of the HIV in the subject to one or both of the first antibody and the second antibody. In some embodiments, the subject is viremic (i.e., HIV-1 RNA >50 copies/mL). In some embodiments, the subject is virologically suppressed (i.e., HIV-1 RNA <50 copies/mL). In some embodiments, the subject is receiving antiretroviral therapy (ART). In some embodiments, antiretroviral therapy (ART) is discontinued before administration of the first and second antibody, e.g., before the first time point. In some embodiments, the subject is acutely infected with HIV. In some embodiments, subject has an HIV infection of Fiebig stage IV or earlier. In some embodiments, the subject has not seroconverted. In some embodiments, the subject is recently infected with HIV. In some embodiments, the antibody is administered to a subject having an HIV infection of Fiebig stage V or Fiebig stage VI. In some embodiments, the subject is chronically infected with HIV. In some embodiments, the subject is infected with HIV clade B viruses.

In another aspect, provided are methods of treating or preventing HIV in a human subject in need thereof. In some embodiments, the methods comprise: (a) Co-administering at a first time point (i) an effective amount of 10-1074-LS (zinlirvimab; GS-2872) and (ii) an effective amount of 3BNC117-LS (teropavimab; GS-5423); and (b) Co-administering at a second time point at least about 24 weeks, e.g., at least about 25 weeks, e.g., at least about 26 weeks, after the first time point an effective amount of 10-1074-LS and an effective amount of 3BNC117-LS. In some embodiments, the 10-1074-LS and the 3BNC117-LS are co-administered every 6 months (Q6M). In some embodiments, the 10-1074-LS and the 3BNC117-LS are co-administered every 24 weeks (Q24W). In some embodiments, the 10-1074-LS and the 3BNC117-LS are co-administered every 25 weeks (Q25W). In some embodiments, the 10-1074-LS and the 3BNC117-LS are co-administered every 26 weeks (Q26W). In some embodiments, the 10-1074-LS and the 3BNC117-LS are co-administered 2 times over 1 year. In some embodiments, the 10-1074-LS and the 3BNC117-LS are co-administered 4 times over 2 years. In some embodiments, the 10-1074-LS and the 3BNC117-LS are co-administered 6 times over 3 years. In some embodiments, the 10-1074-LS and the 3BNC117-LS are co-administered 8 times over 4 years. In some embodiments, the 10-1074-LS is administered intravenously at a dose of 30 mg/kg and the 3BNC117-LS is administered intravenously at a dose of 30 mg/kg. In some embodiments, the 10-1074-LS is administered intravenously at a dose of 10 mg/kg and the 3BNC117-LS is administered intravenously at a dose of 30 mg/kg. In some embodiments, the 10-1074-LS and the 3BNC117 are independently administered intravenously at a dose in the range of from about 500 mg to about 3000 mg, e.g., from about 550 mg to about 2900 mg, e.g., from about 600 mg to about 2800 mg, e.g., from about 650 mg to about 2700 mg, e.g., from about 700 mg to about 2600 mg, e.g., from about 850 mg to about 2550 mg. In some embodiments, the 10-1074-LS is administered intravenously at a dose of 2550 mg and the 3BNC117-LS is administered intravenously at a dose of 2550 mg. In some embodiments, the 10-1074-LS is administered intravenously at a dose of 850 mg and the 3BNC117-LS is administered intravenously at a dose of 1275 mg. In some embodiments, the 10-1074-LS is administered intravenously at a dose of 850 mg and the 3BNC117-LS is administered intravenously at a dose of 1700 mg. In some embodiments, the 10-1074-LS is administered intravenously at a dose of 850 mg and the 3BNC117-LS is administered intravenously at a dose of 2550 mg. In some embodiments, the serum concentration of the 10-1074-LS and the 3BNC117-LS are at least 10 μg/mL at 26 weeks after the first time point. In some embodiments, the plasma or serum concentration of HIV RNA is less than 50 copies/mL at 26 weeks after the first time point. In some embodiments, the methods further comprise co-administering one or more long-acting HIV drugs. In some embodiments, the one or more long-acting HIV drugs are selected from a long-acting capsid inhibitor, a long-acting integrase strand transfer inhibitor (INSTI), a long-acting non-nucleoside reverse transcriptase inhibitor (NNRTI), a long-acting nucleoside reverse transcriptase inhibitors (NRTI), and a long-acting protease inhibitor (PI). In some embodiments, the long-acting capsid inhibitor is selected from lenacapavir, VH4004280 and VH4011499. In some embodiments, the long-acting capsid inhibitor comprises lenacapavir. In some embodiments, the lenacapavir is administered at a dose in the range of 300 mg to 1000 mg. In some embodiments, the lenacapavir is administered orally or subcutaneously. In some embodiments, the long-acting INSTI is selected from bictegravir, raltegravir, elvitegravir, dolutegravir, cabotegravir, GS-1720, GS-6212, GS-1219, GS-3242 and VH4524184. In some embodiments, the long-acting NNRTI is selected from rilpivirine, elsulfavirine, doravirine and GS-5894. In some embodiments, the long-acting NRTI is selected from islatravir and prodrugs thereof, tenofovir alafenamide (TAF) and prodrugs of tenofovir, rovafovir etalafenamide and GS-1614. In some embodiments, the long-acting protease inhibitor is selected from atazanavir, ritonavir, darunavir, GS-1156 and prodrugs of GS-1156, and combinations thereof. In some embodiments, the methods further comprises determining the sensitivity of the HIV in the subject to one or both of 10-1074-LS and 3BNC117-LS. In some embodiments, the subject is viremic. In some embodiments, the subject is virologically suppressed. In some embodiments, the subject is receiving antiretroviral therapy (ART). In some embodiments, antiretroviral therapy (ART) has been discontinued before administration of 10-1074-LS and 3BNC117-LS. In some embodiments, the subject is acutely infected with HIV. In some embodiments, the subject has an HIV infection of Fiebig stage IV or earlier. In some embodiments, the subject has not seroconverted. In some embodiments, the subject is recently infected with HIV. In some embodiments, the antibody is administered to a subject having an HIV infection of Fiebig stage V or Fiebig stage VI. In some embodiments, the subject is chronically infected with HIV. In some embodiments, the subject is infected with HIV clade B viruses.

In a further aspect, provided are kits. In some embodiments, the kits comprise one or more unitary doses of a first antibody that binds HIV gp120 V3 glycan and a second antibody that binds HIV gp120 CD4bs, wherein the first antibody and the second antibody have serum half-life extending amino acid substitutions, and wherein the first antibody and the second antibody are formulated for administration twice annually (e.g., every 6 months (Q6M), every 26 weeks (Q26W), every 25 weeks (Q25W), or every 24 weeks (Q24W)). In some embodiments, the one or more the unitary doses of the first antibody and the second antibody independently are in the range of from about 500 mg to about 3000 mg, e.g., from about 550 mg to about 2900 mg, e.g., from about 600 mg to about 2800 mg, e.g., from about 650 mg to about 2700 mg, e.g., from about 700 mg to about 2600 mg, e.g., from about 850 mg to about 2550 mg. As appropriate. the unitary doses can be the same or different. In some embodiments, the kits comprise one or more unitary doses of 3BNC117-LS (teropavimab; GS-5423) and 10-1074-LS (zinlirvimab; GS-2872), wherein the 3BNC117-LS (teropavimab) and the 10-1074-LS (zinlirvimab) are formulated for administration twice annually (e.g., every 6 months (Q6M), every 26 weeks (Q26W), every 25 weeks (Q25W), or every 24 weeks (Q24W)). In some embodiments, the unitary doses of 10-1074-LS and 3BNC117-LS are independently in the range of from about 500 mg to about 3000 mg, e.g., from about 550 mg to about 2900 mg, e.g., from about 600 mg to about 2800 mg, e.g., from about 650 mg to about 2700 mg, e.g., from about 700 mg to about 2600 mg, e.g., from about 850 mg to about 2550 mg. In some embodiments, the one or more unitary doses of 10-1074-LS are 2550 mg and the one or more unitary doses of 3BNC117-LS are 2550 mg. In some embodiments, the one or more unitary doses of 10-1074-LS are 850 mg and the one or more unitary doses of 3BNC117-LS are 1275 mg. In some embodiments, the one or more unitary doses of 10-1074-LS are 850 mg and the one or more unitary doses of 3BNC117-LS are 1700 mg. In some embodiments, the one or more unitary doses of 10-1074-LS are 850 mg and the one or more unitary doses of 3BNC117-LS are 2550 mg. In some embodiments, the 10-1074-LS and the 3BNC117-LS are formulated for intravenous administration. In some embodiments, the one or more unitary doses are comprised in one or more containers. In some embodiments, the one or more containers are selected from vials, ampules and preloaded syringes. In some embodiments, the kits further comprise one or more unitary doses of one or more long-acting HIV drugs. In some embodiments, the one or more unitary doses of one or more long-acting HIV drugs are selected from a long-acting capsid inhibitor, a long-acting integrase strand transfer inhibitor (INSTI), a long-acting non-nucleoside reverse transcriptase inhibitor (NNRTI), a long-acting nucleoside reverse transcriptase inhibitors (NRTI), and a long-acting protease inhibitor (PI). In some embodiments, the long-acting capsid inhibitor is selected from lenacapavir, VH4004280 and VH4011499. In some embodiments, the long-acting capsid inhibitor comprises lenacapavir. In some embodiments, the unitary dose of lenacapavir is in the range of 300 mg to 1000 mg. In some embodiments, the lenacapavir is formulated for oral or subcutaneous administration. In some embodiments, the long-acting INSTI is selected from bictegravir, raltegravir, elvitegravir, dolutegravir, cabotegravir, GS-1720, GS-6212, GS-1219, GS-3242 and VH4524184. In some embodiments, the long-acting NNRTI is selected from rilpivirine, elsulfavirine, doravirine and GS-5894. In some embodiments, the long-acting NRTI is selected from islatravir and prodrugs thereof, tenofovir alafenamide (TAF) and prodrugs of tenofovir, rovafovir etalafenamide and GS-1614. In some embodiments, the long-acting protease inhibitor is selected from atazanavir, ritonavir, darunavir, GS-1156 and prodrugs of GS-1156, and combinations thereof.

### BRIEF DESCRIPTION OF THE DRAWINGS

FIGS. 1A-1C. FIG. 1A illustrates a study schema of the Phase 1b study GS-US-536-5816 (NCT04811040 on ClinicalTrials.gov). FIG. 1B illustrates the participant disposition. All randomized participants were included in the safety analysis (N=21); those who received the complete study regimens (oral LEN, SC LEN, and bNAbs) are included in the efficacy analyses (N=20). FIG. 1C illustrates virologic efficacy outcomes at Week 26 of the Phase 1b study by FDA snapshot algorithm. 18 of 20 participants maintained viral suppression on study regimen through Week 26. One participant withdrew at Week 12 with HIV-1 RNA <50 copies/mL. One participant had a confirmed virologic rebound at Week 16 and was resuppressed on baseline oral ART.

FIG. 2 illustrates pharmacokinetics of teropavimab (TAB), zinlirvimab (ZAB) and lenacapavir (LEN) in the Phase 1b study.

FIGS. 3A-3D illustrates simulated Cₘₐₓ (Figs. A and B) and Cₘᵢₙ (Figs. C and D) at Week 26 after IV administration of 30 mg/kg or 2550 mg GS-5423 (Figs. A and C) and 10 mg/kg, 30 mg/kg, 850 mg, or 2550 mg GS-2872 (Figs. B and D) every 6 months. Box: interquartile range, horizontal line: median, whisker: 1.5 times interquartile range, not exceeding the minimum and maximum values, dots: outliers.

FIGS. 4A-4B illustrates simulated median (line) and 5th-95th percentiles (shaded area) GS-5423 (teropavimab) (FIG. 3A) and GS-2872 (zinlirvimab) (FIG. 3B) concentration-time profiles at different doses given every 6 months.

FIG. 5 illustrates a schema of a PK-PD viral dynamic model for evaluation of GS-5423 (3BNC117-LS; teropavimab; TAB) and GS-2872 (10-1074-LS; zinlirvimab; ZAB) concentrations and prediction of washout duration. C₁ and C₂, serum concentration of 3BNC117/TAB and 10-1074/ZAB, respectively; EC_{50,drug,1} and EC_{50,drug2}, concentration that leads to 50% maximum effect of 3BNC117/TAB and 10-1074/ZAB, respectively; fi, initial fraction of i^{th} viral compartment; k_{g}, maximal viral replication rate constant; k_{del,drug1} and k_{del,drug2}, viral elimination rate constant for 3BNC117/TAB and 10-1074/ZAB, respectively; r_{d,i}, viral elimination rate for i^{th} viral compartment; r_{g,i}, viral replication rate for i^{th} viral compartment; TAB, teropavimab; VL₁, copies of viruses sensitive to both 3BNC117/TAB and 10-1074/ZAB; VL₂, copies of viruses sensitive to 3BNC117/TAB and resistant to 10-1074/ZAB; VL₃, copies of viruses sensitive to 10-1074/ZAB and resistant to 3BNC117/TAB; VL₄, copies of viruses resistant to both 3BNC117/TAB and 10-1074/ZAB (assumed to be 0); VLₜₒₜₐₗ, total viral load; VLₛₛ, steady state viral load; ZAB, zinlirvimab.

FIG. 6 illustrates observed vs predicted bNAb serum concentrations from the PK models. bNAb, broadly neutralizing antibody; PK, pharmacokinetic; TAB, teropavimab; ZAB, zinlirvimab. Circles represent individual data. Solid lines represent LOESS (locally estimated scatterplot smoothing) fit. Dashed lines represent the line of identity.

FIG. 7 illustrates model-predicted PK profiles after single-dose 30 mg/kg IV infusion. IV, intravenous; PWH, people with HIV. 1000 virtual subjects were simulated using the population PK models of 3BNC117, 10-1074, TAB, and ZAB. Solid and dashed lines represent model-predicted medians for mono and combination therapy, respectively, and shaded areas represent the 90% prediction intervals of the population.

FIG. 8 illustrates model-predicted vs observed viral dynamics after bNAb treatment in viremic people with HIV. Q5, 5th percentile; Q50, 50th percentile; Q95, 95th percentile. 100 trial simulations were performed with the same number of subjects as the original dataset used for modeling fitting. The predicted quantiles were calculated from the median of the quantiles across all trial replicates. Arrows represent bNAb(s) dosing.

FIG. 9 illustrates model-predicted vs observed time to viral rebound during ATI after bNAb treatment. ATI, analytical treatment interruption; bNAb, broadly neutralizing antibody; CI, confidence interval. Doses in the ATI studies: NCT02446847, 2 doses of 30 mg/kg 3BNC117 every 3 weeks or up to 4 doses of 30 mg/kg 3BNC117 every 2 weeks; NCT02825797, up to 3 doses of 30 mg/kg 3BNC117 and 30 mg/kg 10-1074 every 3 weeks; NCT03526848, 30 mg/kg 3BNC117 and 30 mg/kg 10-1074 every 2 weeks for 3 doses followed by every 4 weeks for up to 4 doses (group 1, ATI started on day 2; group 2, ATI started at week 26 [1 participant started at week 21]). 100 trial simulations were performed with the same number of subjects as the original dataset used for model fitting. Solid blue lines (shaded region) represent the medians (2.5th to 97.5th percentiles) across all trial replicates. Arrows represent bNAb(s) dosing. Red dotted lines indicate start of ATI.

FIG. 10 illustrates model-predicted viral rebound dynamics after single dose TAB/ZAB combination treatment with different ATI start times. PD, pharmacodynamic. Dotted horizontal lines indicate the threshold for viral rebound (200 cp/mL). 1000 virtual subjects were simulated using the population PK-PD model. Solid lines represent model-predicted median, and shaded areas represent the 90% prediction intervals of the population. Arrows represent bNAb(s) dosing. Red dashed lines indicate start of ATI.

FIG. 11 illustrates simulated bNAb serum concentrations and their ratios over in vivo EC₅₀ over time after single-dose TAB 30 mg/kg and ZAB 10 mg/kg IV administration. EC₅₀, concentration that leads to 50% maximum drug effect. 1000 virtual subjects were simulated using the population PK models. Solid lines represent model-predicted medians, and shaded areas represent the 90% prediction intervals of the population. Ratios were calculated based on the estimated EC₅₀ values from the PK-PD model (25.4 μg/mL for TAB, 32.2 μg/mL for ZAB). Black dashed lines indicate the proposed earliest start time of ATI.

FIG. 12 illustrates a study schema of the Phase 2 study GS-US-539-5939.

### DETAILED DESCRIPTION

1. Introduction

Accordingly, the present methods are based, in part, on the discovery that co-administration of a first anti-HIV broadly neutralizing antibody (bNAb) that binds to an epitope of gp120 within the third variable loop (V3) and/or high mannose patch comprising a N332 oligomannose glycan and a second bNAb that binds to an epitope of gp120 comprising the CD4 binding site (CD4bs) having Fc amino acid substitutions that extend serum half-life can be administered twice annually (e.g., Q6M, Q24W, Q25W, Q26W), and achieve therapeutic efficacy. To date, bNAbs, even having serum half-life extending Fc amino acid substitutions have been administered every 3 months or more often.

Generally, the methods entail co-administering at a first time point (i) an effective amount of a first antibody that competes with or comprises VH and VL regions that bind to an epitope of gp120 within the third variable loop (V3) and/or high mannose patch comprising a N332 oligomannose glycan and (ii) an effective amount of a second antibody that competes with or comprises VH and VL regions that bind to an epitope of gp120 comprising the CD4 binding site (CD4bs), wherein the first antibody and the second antibody both comprise Fc amino acid substitutions to extend serum half-life; and then co-administering at a second time point at least about 24 weeks, e.g., at least about 25 weeks, e.g., at least about 26 weeks, after the first time point an effective amount of the first antibody and an effective amount of the second antibody.

3BNC117 and 10-1074 have undergone modifications to increase the half-lives, resulting in GS 5423 (teropavimab; 3BNC117-LS) and GS-2872 (zinlirvimab; 10-1074-LS) and allowing for the maintenance of high bNAb concentrations over long durations. Combination therapy consisting of long-acting bNAbs with an ARV drug may overcome the limitations of bNAbs alone and enable a safe long-acting treatment option for PWH. The modified LS versions contain two amino acid substitutions in the Fc: methionine to leucine at Fc position 428 (M428L), and asparagine to serine at Fc position 434 (N434S) (EU numbering). These substitutions enhance the antibody binding affinity to the neonatal Fc receptor (FcRn), prolonging the bNAbs' half-life in vivo. Affinity binding to other Fc receptors remains unchanged. These modifications do not alter the fragment antigen-binding domain (Fab) of the bNAbs and therefore do not alter their interaction with antigen or safety profile.

2. Co-Administered Broadly Neutralizing Antibodies

a. Broadly Neutralizing Antibodies, Generally

HIV-1 is the main family of HIV and accounts for 95% of all infections worldwide. HIV-2 is mainly seen in a few West African countries.

HIV viruses are divided into specific groups, M, N, O and P, of which M is the “major” group and responsible for majority of HIV/AIDS globally. Based on their genetic sequence, Group M is further subdivided into subtypes (also called clades) with prevalence in distinct geographical locations.

A Group M “subtype” or “clade” is a subtype of HIV-1 group M defined by genetic sequence data. Examples of Group M subtypes include Subtypes A-K. Some of the subtypes are known to be more virulent or are resistant to different medications. There are also “circulating recombinant forms” or CRFs derived from recombination between viruses of different subtypes, which are each given a number. CRF12_BF, for example, is a recombination between subtypes B and F. Subtype A is common in West Africa. Subtype B is the dominant form in Europe, the Americas, Japan, Thailand, and Australia. Subtype C is the dominant form in Southern Africa, Eastern Africa, India, Nepal, and parts of China. Subtype D is generally only seen in Eastern and central Africa. Subtype E has never been identified as a nonrecombinant, only recombined with subtype A as CRF01_AE. Subtype F has been found in central Africa, South America and Eastern Europe. Subtype G (and the CRF02_AG) have been found in Africa and central Europe. Subtype H is limited to central Africa. Subtype I was originally used to describe a strain that is now accounted for as CRF04_cpx, with the cpx for a “complex” recombination of several subtypes. Subtype J is primarily found in North, Central and West Africa, and the Caribbean Subtype K is limited to the Democratic Republic of Congo and Cameroon. These subtypes are sometimes further split into sub-subtypes such as A1 and A2 or F1 and F2. In 2015, the strain CRF19, a recombinant of subtype A, subtype D, and subtype G, with a subtype D protease was found to be strongly associated with rapid progression to AIDS in Cuba.

This disclosure provides, inter alia, methods entailing administration of human anti-HIV neutralizing antibodies (e.g., broadly neutralizing Abs) that target the gp120 polypeptide on the surface of HIV-infected cells. Neutralizing antibodies against viral envelope proteins provide adaptive immune defense against HIV-1 exposure by blocking the infection of susceptible cells. Broad neutralization indicates that the antibodies can neutralize HIV-1 isolates from different clades. Thus, the anti-HIV gp120 binding antibodies described herein have cross-clade binding activity.

In certain embodiments, the administered antibody is or is derived from human neutralizing antibodies (e.g., monoclonal) that target HIV-1. A “neutralizing antibody” is one that can neutralize the ability of HIV to initiate and/or perpetuate an infection in a host and/or in target cells in vitro. The disclosure provides neutralizing monoclonal human antibodies, wherein the antibody recognizes an antigen from HIV, e.g., a gp120 polypeptide. In certain embodiments, a “neutralizing antibody” may inhibit the entry of HIV-1 virus, e.g., SF162 and/or JR-CSF, with a neutralization index >1.5 or >2.0 (Kostrikis L G et al., J. Virol., 70(1): 445-458 (1996)).

In some embodiments, the administered antibody is or is derived from human broadly neutralizing antibodies (e.g., monoclonal) that target HIV-1. By “broadly neutralizing antibodies” are meant antibodies that neutralize more than one HIV-1 virus species (from diverse clades and different strains within a clade) in a neutralization assay. A broadly neutralizing antibody may neutralize at least 2, 3, 4, 5, 6, 7, 8, 9 or more different strains of HIV-1, the strains belonging to the same or different clades. In particular embodiments, a broad neutralizing antibody may neutralize multiple HIV-1 species belonging to at least 2, 3, 4, 5, or 6 different clades. In certain embodiments, the inhibitory concentration of the anti-HIV gp120 V3 glycan binding antibody or antigen-binding fragment may be less than about 0.0001 μg/ml, less than about 0.001 μg/ml, less than about 0.01 μg/ml, less than about 0.1 μg/ml, less than about 0.5 μg/ml, less than about 1.0 μg/ml, less than about 5 μg/ml, less than about 10 μg/ml, less than about 25 μg/ml, less than about 50 μg/ml, or less than about 100 μg/ml to neutralize about 50% of the input virus in the neutralization assay.

gp120

Envelope glycoprotein gp120 (or gp120) is a 120 kDa glycoprotein that is part of the outer layer of HIV. It presents itself as viral membrane spikes consisting of three molecules of gp120 linked together and anchored to the membrane by gp41 protein. Gp120 is essential for viral infection as it facilitates HIV entry into the host cell through its interaction with cell surface receptors. These receptors include DC-SIGN, Heparan Sulfate Proteoglycan, and the CD4 receptor. Binding to CD4 on helper T-cells induces the start of a cascade of conformational changes in gp120 and gp41 that lead to the fusion of the virus with the host cell membrane.

Gp120 is encoded by the HIV env gene. The env gene encodes a gene product of around 850 amino acids. The primary env product is the protein gp160, which gets cleaved to gp120 (about 480 amino acids) and gp41 (about 345 amino acids) in the endoplasmic reticulum by the cellular protease furin.

Broadly neutralizing antibodies are reviewed, e.g., in Walsh and Seaman, Front Immunol. (2021) 12:712122; Julg and Barouch, Semin Immunol. (2021) 51:101475; Hsu, et al., Front Immunol. (2021) 12:710044; Karuna and Corey, Annu Rev Med. (2020) 71:329-346; Haynes, et al., Sci Transl Med. (2019) 11(516):eaaz2686; Dashti, et al., Trends Mol Med. (2019) 25(3):228-240; McCoy, Retrovirology (2018) 15:70; Sok and Burton, Nat Immunol. 2018 19(11):1179-1188; Possas, et al., Expert Opin Ther Pat. 2018 July; 28(7):551-560; and Stephenson and Barouch, Curr HIV/AIDS Rep (2016) 13:31-37, which are hereby incorporated herein by reference in their entirety for all purposes.

b. Antibodies Directed to the V3 Glycan Region of HIV gp120

The V3 glycan site on gp120 is formed partly by a section of the CCR5 co-receptor site and partly by the surrounding camouflaging glycans (so-called “high mannose patch”) (Sok, et al., Immunity (2016) 45, 31-45). Broadly neutralizing antibodies (bnAbs) to the V3 glycan site are the most common of all Abs found in HIV infection (Walker, et al., PLoS Pathog. (2010) 6:e1001028 (2010); Landais, et al., PLoS Pathog. (2016) 12:e1005369; Georgiev, et al. Science (2013) 340:751-756). A consensus sequence of the V3 region of gp120 (Milich et al., J Virol., 67(9):5623-5634 (1993) is provided below:

(SEQ ID NO: 1)
CTRPNNNTRKSIHIGPGRAFYTTGEIIGDIRQAHC.

The amino acid sequence of an exemplary gp160 polypeptide of HIV clone WITO is provided below (the V3 hypervariable loop is boldened and the N332 potential N-linked glycosylation site is boldened and underlined):

(SEQ ID NO: 2)
MKVMGTKKNYQHLWRWGIMLLGMLMMSSAAEQLWVTVYYGVPVWREANTTLFCASDAKAYDTEV
HNVWATHACVPTDPNPQEVVMGNVTEDFNMWKNNMVEQMHEDIISLWDQSLKPCVKLTPLCVTL
HCTNVIISSINGSTANVIMREEMKNCSENTTIVIRDKIQKEYALFYKLDIVPIEGKNINTSYRL
INCNTSVITQACPKVSFEPIPIHYCAPAGFAILKCNNKTENGKGPCRNVSTVQCTHGIKPVVST
QLLLNGSLAEEDIIIRSENFINNGKNIIVQLKEPVKINCTRPGNNTRRSINIGPGRAFYATGAI
IGDIRKAHCNISTEQWNNTLTQIVDKLREQFGNKTIIFNQSSGGDPEVVMHTENCGGEFFYCNS
TQLFNSTWENNGISTWNSTADNITLPCRIKQVINMWQEVGKAMYAPPIRGQIDCSSNITGLILT
RDGGSNSSQNETFRPGGGNMKDNWRSELYKYKVVKIEPLGIAPTRAKRRVVQREKRAVTLGAVE
LGFLGAAGSTMGAASLILTVQARLLLSGIVQQQSNLLRAIEAQQHMLQLTVWGIKQLQARVLAI
ERYLKDQQLLGIWGCSGKLICITTVPWNISWSNKSYDYIWNNMTWMQWEREIDNYTGFIYTLIE
ESQNQQEKNELELLELDKWASLWNWFNITNWLWYIKLFIMIIGGLVGLRIVCAVLSIVNRVRQG
YSPLSFQTRLPNPRGPDRPEETEGEGGERDRDRSARLVNGFLAIIWDDLRSLCLFSYHRLRDLL
LIVARVVEILGRRGWEILKYWWNLLKYWSQELKNSAVSLLNVTAIAVAEGTDRVIEIVQRAVRA
ILHIPTRIRQGFERALL

The amino acid sequence of an exemplary gp160 polypeptide of HIV clone identified in NCBI Ref Seq No. NP_057856.1 is provided below (the V3 hypervariable loop is boldened and the N332 potential N-linked glycosylation site is boldened and underlined):

(SEQ ID NO: 3)
MRVKEKYQHLWRWGWRWGTMLLGMLMICSATEKLWVTVYYGVPVWKEATTTLFCASDAKAYDTE
VHNVWATHACVPTDPNPQEVVLVNVTENFNMWKNDMVEQMHEDIISLWDQSLKPCVKLTPLCVS
LKCTDLKNDTNTNSSSGRMIMEKGEIKNCSFNISTSIRGKVQKEYAFFYKLDIIPIDNDTTSYK
LTSCNTSVITQACPKVSFEPIPIHYCAPAGFAILKCNNKTENGTGPCTNVSTVQCTHGIRPVVS
TQLLLNGSLAEEEVVIRSVNFTDNAKTIIVQLNISVEINCTRPNNNTRKRIRIQRGPGRAFVTI
GKIGNMRQAHCNISRAKWNNTLKQIASKLREQFGNNKTIIFKQSSGGDPEIVTHSENCGGEFFY
CNSTQLENSTWENSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKRA
VGIGALFLGFLGAAGSTMGAASMILTVQARQLLSGIVQQQNNLLRAIEAQQHLLQLTVWGIKQL
QARILAVERYLKDQQLLGIWGCSGKLICITAVPWNASWSNKSLEQIWNHITWMEWDREINNYTS
LIHSLIEESQNQQEKNEQELLELDKWASLWNWENIINWLWYIKLFIMIVGGLVGLRIVFAVLSI
VNRVRQGYSPLSFQTHLPTPRGPDRPEGIEEEGGERDRDRSIRLVNGSLALIWDDLRSLCLFSY
HRLRDLLLIVTRIVELLGRRGWEALKYWWNLLQYWSQELKNSAVSLLNATAIAVAEGTDRVIEV
VQGACRAIRHIPRRIRQGLERILL

The amino acid sequence of an exemplary gp120 polypeptide of HXB2 subtype B HIV-1 isolate (GenBank Accession No. K0345; corresponding to residues 1-511 of NCBI Ref Seq No. NP_057856.1) is provided below (the V3 hypervariable loop is boldened and the N332 potential N-linked glycosylation site is boldened and underlined; signal peptide is underlined):

(SEQ ID NO: 4)
MRVKEKYQHLWRWGWRWGTMLLGMLMICSATEKLWVTVYYGVPVWKEATTTLFCASDAKAYDTE
VHNVWATHACVPTDPNPQEVVLVNVTENFNMWKNDMVEQMHEDIISLWDQSLKPCVKLTPLCVS
LKCTDLKNDTNINSSSGRMIMEKGEIKNCSFNISTSIRGKVQKEYAFFYKLDIIPIDNDTTSYK
LISCNISVITQACPKVSFEPIPIHYCAPAGFAILKCNNKTENGTGPCTNVSTVQCTHGIRPVVS
TQLLLNGSLAEEEVVIRSVNFTDNAKTIIVQLNISVEINCTRPNNNTRKRIRIQRGPGRAFVTI
GKIGNMRQAHCNISRAKWNNTLKQIASKLREQFGNNKTIIFKQSSGGDPEIVTHSENCGGEFFY
CNSTQLFNSTWENSTWSTEGSNNTEGSDIITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR

The amino acid sequence of an exemplary gp120 polypeptide is provided below:

(SEQ ID NO: 5)
AEQLWVTVYYGVPVWREANTTLFCASDAKAYDTEVHNVWATHACVPTDPNPQEVVMGNVTEDEN
MWKNNMVEQMHEDIISLWDQSLKPCVKLTPLCVTLHCTNVTISSINGSTANVTMREEMKNCSEN
TTIVIRDKIQKEYALFYKLDIVPIEGKNINTSYRLINCNTSVITQACPKVSFEPIPIHYCAPAG
FAILKCNNKTFNGKGPCRNVSTVQCTHGIKPVVSTQLLINGSLAEEDIIIRSENFINNGKNIIV
QLKEPVKINCTRPGNNTRRSINIGPGRAFYATGAIIGDIRKAHCNISTEQWNNTLIQIVDKLRE
QFGNKTIIFNQSSGGDPEVVMHTFNCGGEFFYCNSTQLENSTWENNGTSTWNSTADNITLPCRI
KQVINMWQEVGKAMYAPPIRGQIDCSSNITGLILTRDGGSNSSQNETFRPGGGNMKDNWRSELY
KYKVVKIEPLGIAPTRAKRRVVQREKR.

The amino acid sequence of another exemplary gp120 polypeptide (see, bioafrica.net/proteomics/ENV-GP120prot.html) is provided below:

(SEQ ID NO: 6)
TEKLWVTVYY GVPVWKEATT TLFCASDAKA YDTEVHNVWA
THACVPTDPN PQEVVLVNVT ENFNMWKNDM VEQMHEDIIS
LWDQSLKPCV KLTPLCVSLK CTDLKNDTNT NSSSGRMIME
KGEIKNCSEN ISTSIRGKVQ KEYAFFYKLD IIPIDNDTTS
YKLTSCNTSV ITQACPKVSF EPIPIHYCAP AGFAILKCNN
KTFNGIGPCT NVSTVQCTHG IRPVVSTQLL LNGSLAEEEV
VIRSVNETDN AKTIIVQLNT SVEINCTRPN NNTRKRIRIQ
RGPGRAFVTI GKIGNMRQAH CNISRAKWNN TLKQIASKLR
EQFGNNKTII FKQSSGGDPE IVTHSENCGG EFFYCNSTQL
FNSTWENSTW STEGSNNTEG SDTITLPCRI KQIINMWQKV
GKAMYAPPIS GQIRCSSNIT GLLLTRDGGN SNNESEIFRP
GGGDMRDNWR SELYKYKVVK IEPLGVAPTK AKRRVVQREK R

Genomic diversity among independent human immunodeficiency virus type 1 (HIV-1) isolates, to a lesser degree among sequential isolates from the same patients, and even within a single patient isolate is a well-known feature of HIV-1. Although this sequence heterogeneity is distributed throughout the genome, most of the heterogeneity is located in the env gene. Comparison of predicted amino acid sequences from several different isolates has shown that sequence heterogeneity is clustered in five variable regions (designated V1 through V5) of the surface glycoprotein, gp120. The V3 region, although only 35 amino acids long, exhibits considerable sequence variability. Interestingly, despite this variability, the V3 region includes determinants that mediate interactions with CD4⁺ cells. The increase in gp120 variability results in higher levels of viral replication, suggesting an increase in viral fitness in individuals infected by diverse HIV-1 variants. Variability in potential N-linked glycosylation sites (PNGSs) also result in increased viral fitness. PNGSs allow for the binding of long-chain carbohydrates to the high variable regions of gp120. Thus, the number of PNGSs in env might affect the fitness of the virus by providing more or less sensitivity to neutralizing antibodies.

Illustrative broadly neutralizing antibodies that bind to gp120 in the third variable loop (V3) and/or high mannose patch comprising a N332 oligomannose glycan and which can be used in the herein described methods include without limitation GS-9722 (elipovimab), GS-9721, PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-LS (zinlirvimab; GS-2872), 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. Additional broadly neutralizing antibodies that bind to gp120 in the third variable loop (V3) and/or high mannose patch comprising a N332 oligomannose glycan and which can be used in the herein described methods are described, e.g., in WO 2012/030904; WO 2014/063059; WO 2016/149698; WO 2017/106346; WO 2018/075564, WO 2018/125813; WO 2018/237148, WO 2019/226829, WO 2020/023827, WO2020/056145 and Kerwin, et al., J Pharm Sci. 2020 January; 109(1):233-246, which are hereby incorporated herein by reference in their entireties for all purposes.

Illustrative sequences of complementarity determining regions (CDRs) of the antibody targeting HIV gp120 V3 glycan region, are provided in Tables A1-A4. Illustrative sequences of the VH and VL of the antibody targeting HIV gp120 V3 glycan region, are provided in Table B.

TABLE A1
CDRs (Kabat) for Anti-HIV gp120 V3 Glycan-Binding antibodies

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 1 | DSYWS | YVHKSGDTNYSPSLKS | TLHGRRIYGIVAFNEW | GEKSLGSRAVQ | NNQDRPS | HIWDSRVPTKWV |
| | SEQ ID | SEQ ID NO: 8 | FTYFYMDV | SEQ ID | SEQ ID | SEQ ID NO: 12 |
| | NO: 7 | | SEQ ID NO: 9 | NO: 10 | NO: 11 | |
| 2 | DSYWS | YVHKSGDTNYNPSLKS | TLHGPRIYGIVAFNEW | GEKSLGSRAVQ | NNQDPPS | HIWDSRVPTKWV |
| | SEQ ID | SEQ ID NO: 13 | FTYFYMDV | SEQ ID | SEQ ID | SEQ ID NO: 12 |
| | NO: 7 | | SEQ ID NO: 9 | NO: 10 | NO: 11 | |
| 3 | NYYWT | YISDRESATYNPSLNS | ARRGQRIYGVVSFGEF | GRQALGSRAVQ | NNQDRPS | HMWDSRSGFSWS |
| | SEQ ID | SEQ ID NO: 15 | FYYYSMDV | SEQ ID | SEQ ID | SEQ ID NO: 18 |
| | NO: 14 | | SEQ ID NO: 16 | NO: 17 | NO: 11 | |
| 4 | NYYWT | YISDRETTTYNPSLNS | ARRGQRIYGVVSFGEE | GRQALGSRAVQ | NNQDRPS | AMWDSRSGESWS |
| | SEQ ID | SEQ ID NO: 19 | FYYYYMDV | SEQ ID | SEQ ID | SEQ ID NO: 18 |
| | NO: 14 | | SEQ ID NO: 20 | NO: 17 | NO: 11 | |
| 5 | GREWS | YESDTDRSEYNPSLRS | AQQGKRIYGIVSEGEF | GERSRGSRAVQ | NNQDRPA | HYWDSRSPISWI |
| | SEQ ID | SEQ ID NO: 22 | FYYYYMDA | SEQ ID | SEQ ID | SEQ ID NO: 26 |
| | NO: 21 | | SEQ ID NO: 23 | NO: 24 | NO: 25 | |
| 6 | GRFWS | YESDTDRSEYNPSLRS | AQQGKRIYGIVSFGEL | GERSRGSRAVQ | NNQDRPA | HYWDSRSPISWI |
| | SEQ ID | SEQ ID NO: 22 | FYYYYMDA | SEQ ID | SEQ ID | SEQ ID NO: 26 |
| | NO: 21 | | SEQ ID NO: 27 | NO: 24 | NO: 25 | |
| 7 | DNYWS | YVHDSGDTNYNPSLKS | TKHGRRIYGVVAFKEW | GEESLGSRSVI | NNNDRPS | HIWDSRRPTNWV |
| | SEQ ID | SEQ ID NO: 29 | FTYFYMDV | SEQ ID | SEQ ID | SEQ ID NO: 33 |
| | NO: 28 | | SEQ ID NO: 30 | NO: 31 | NO: 32 | |
| 8 | DAYWS | YVHHSGDTNYNPSLKR | ALAGKRIYGIVALGEL | GKESIGSRAVQ | NNQDRPA | HIYDARGGINWV |
| | SEQ ID | SEQ ID NO: 35 | FTYFYNDV | SEQ ID | SEQ ID | SEQ ID NO: 38 |
| | NO: 34 | | SEQ ID NO: 36 | NO: 37 | NO: 25 | |
| 9 | ACTYFWG | SLSHCQSFWGSGWTFH | EDGEVLVYNHWPKPAW | NGTATNEVS | GVDKRPP | GSLVGNWDVI |
| | SEQ ID | NPSLKS | VDE | SEQ ID | SEQ ID | SEQ ID NO: 44 |
| | NO: 39 | SEQ ID NO: 40 | SEQ ID NO: 41 | NO: 42 | NO: 43 | |
| 10 | ACDYFWG | GLSHCAGYYNTGWTYH | FDGEVLVYHDWPKPAW | TGTSNREVS | GVNKRPS | SSLVGNWDVI |
| | SEQ ID | NPSLKS | VDL | SEQ ID | SEQ ID | SEQ ID NO: 50 |
| | NO: 45 | SEQ ID NO: 46 | SEQ ID NO: 47 | NO: 48 | NO: 49 | |
| 11 | ACDYFWG | SLSHCAGYYNSGWTYH | FGGDVLVYHDWPKPAW | TGNINNEVS | GVNKRPS | GSLAGNWDVV |
| | SEQ ID | NPSLKS | VDL | SEQ ID | SEQ ID | SEQ ID NO: 54 |
| | NO: 45 | SEQ ID NO: 51 | SEQ ID NO: 52 | NO: 53 | NO: 49 | |
| 12 | ACNSFWG | SLSHCASYWNRGWTYH | FGGEVLRYTDWPKPAW | TGTSNNEVS | DVNKRPS | GSLVGNWDVI |
| | SEQ ID | NPSLKS | VDL | SEQ ID | SEQ ID | SEQ ID NO: 44 |
| | NO: 55 | SEQ ID NO: 56 | SEQ ID NO: 57 | NO: 58 | NO: 59 | |
| 13 | GCDYFWG | GLSHCAGYYNTGWTYH | FDGEVLVYNDWPKPAW | TGTSNNFVS | GVNKRPS | GSLVGNWDVI |
| | SEQ ID | NPSLKS | VDL | SEQ ID | SEQ ID | SEQ ID NO: 44 |
| | NO: 60 | SEQ ID NO: 46 | SEQ ID NO: 61 | NO: 58 | NO: 49 | |
| 14 | TGHYYWG | HIHYTTAVLANPSLKS | SGGDILYYYEWOKPHW | NGTSSDIGGWN | EVNERPS | SSLFGRWDVV |
| | SEQ ID | SEQ ID NO: 63 | FSP | EVS | SEQ ID | SEQ ID NO: 67 |
| | NO: 62 | | SEQ ID NO: 64 | SEQ ID | NO: 66 | |
| | | | | NO: 65 | | |
| 15 | GTDWGENDFHY | SIHWRGRTTHYKTSFR | HKYHDIFRVVPVAGWE | PASQNVKNNLA | DASSRAG | QQYEEWPRT |
| | G | S | DP | SEQ ID | SEQ ID | SEQ ID NO: 73 |
| | SEQ ID | SEQ ID NO: 69 | SEQ ID NO: 70 | NO: 71 | NO: 72 | |
| | NO: 68 | | | | | |
| 16 | GGEWGDSDYHW | SIHWRGTTHYNAPERG | HKYHDIVMVVPIACWE | RASQSVKNNLA | DTSSRAS | QQYEEWPRT |
| | G | SEQ ID NO: 75 | DP | SEQ ID | SEQ ID | SEQ ID NO: 73 |
| | SEQ ID | | SEQ ID NO: 76 | NO: 77 | NO: 78 | |
| | NO: 74 | | | | | |
| 17 | GGEWGDKDYHW | SIHWRGTTHYKESLRR | HRHHDVEMLVPIAGWE | RASQNINKNLA | ETYSKIA | QQYEEWPRT |
| | G | SEQ ID NO: 80 | DV | SEQ ID | SEQ ID | SEQ ID NO: 73 |
| | SEQ ID | | SEQ ID NO: 81 | NO: 82 | NO: 83 | |
| | NO: 79 | | | | | |
| 18 | SDHSWT | DIHYNGATTYNPSLRS | NAIRIYGVVALGEWFH | SGAPLTSRETY | RSSQRSS | QSSDTSDSYKM |
| | SEQ ID | SEQ ID NO: 85 | YGMDV | SEQ ID | SEQ ID | SEQ ID NO: 89 |
| | NO: 84 | | SEQ ID NO: 86 | NO: 87 | NO: 88 | |
| 19 | SDHSWT | DVHYNGDNTYNPSLRG | NVIRVFGVISLGEWFH | SGPPLASRYTY | RDRQFPS | QSSDTSDSYKM |
| | SEQ ID | SEQ ID NO: 90 | YGMDV | SEQ ID | SEQ ID | SEQ ID NO: 89 |
| | NO: 84 | | SEQ ID NO: 91 | NO: 92 | NO: 93 | |
| 20 | SDHSWT | DVHYNGDTTYNPSLRG | NVIRVFGVISIGEWFH | SGPPLASRYTY | RDRQFPS | QSSDTSDSYKM |
| | SEQ ID | SEQ ID NO: 94 | YGMDV | SEQ ID NO: | SEQ ID | SEQ ID NO: 89 |
| | NO: 84 | | SEQ ID NO: 91 | 92 | NO: 93 | |
| 21 | SDHSWT | DIHYNGATTYNPSLRS | NAIRIYGVVALGEWFH | SGAALTSRFTY | RTSQRSS | QSSDTSDSYKM |
| | SEQ ID | SEQ ID NO: 85 | YGMDV | SEQ ID | SEQ ID | SEQ ID NO: 89 |
| | NO: 84 | | SEQ ID NO: 86 | NO: 95 | NO: 96 | |
| 22 | SDHSWT | DIHYGGDITYNPSLRS | NVIRVEGVIALGEWFH | SGPPLASRYCY | RDRQFSS | QSSDINDSYKM |
| | SEQ ID | SEQ ID NO: 97 | YGMDV | SEQ ID | SEQ ID | SEQ ID |
| | NO: 84 | | SEQ ID NO: 98 | NO: 99 | NO: 100 | NO: 101 |
| 23 | SDHSWT | DIHYGGDITYNPSLRS | NVIRVFGVIALGEWFH | SGPPLASRYCY | RDRQFSS | QSSDTSDSFKM |
| | SEQ ID | SEQ ID NO: 97 | YGMDV | SEQ ID | SEQ ID | SEQ ID |
| | NO: 84 | | SEQ ID NO: 98 | NO: 99 | NO: 100 | NO: 102 |
| 24 | SDHSWT | DIHYGGDITYNPSLRS | NVIRVEGVIALGEWFH | SGPPLATRYCY | RDRQFSS | QSSDTSDSYKM |
| | SEQ ID | SEQ ID NO: 97 | YGMDV | SEQ ID | SEQ ID | SEQ ID NO: 89 |
| | NO: 84 | | SEQ ID NO: 98 | NO: 103 | NO: 100 | |
| 25 | SDHSWT | DIHYNGDKTYNPSLRG | NVIRVFGVISLGEWFH | SGPPLASRYTY | RDRQFPS | QSSDTSDSYKM |
| | SEQ ID | SEQ ID NO: 104 | YGMDV | SEQ ID | SEQ ID | SEQ ID NO: 89 |
| | NO: 84 | | SEQ ID NO: 91 | NO: 92 | NO: 93 | |
| 26 | SDHSWT | DIHYGGDITYNPSLRS | NVIRVEGVIALGEWFH | SGPPLASRYCY | RDRQFSS | QSSDNSDSFKM |
| | SEQ ID | SEQ ID NO: 97 | YCMDV | SEQ ID | SEQ ID | SEQ ID |
| | NO: 84 | | SEQ ID NO: 98 | NO: 99 | NO: 100 | NO: 105 |
| 27 | DYAMA | FMRGWAYGGSAQFAAF | EQRNKDYRYGQEGFGY | RASHFIANYVN | ESSTLQR | QQSHSPPVT |
| | SEQ ID | AVG | SYGMDV | SEQ ID | SEQ ID | SEQ ID |
| | NO: 106 | SEQ ID NO: 107 | SEQ ID NO: 108 | NO: 109 | NO: 110 | NO: 111 |
| 28 | DYAMA | FIRGWAYGQAAQYGKS | EQRGGDGRYSGDGFGY | RASHFIANYVN | QSWTLNR | QQSASPPLS |
| | SEQ ID | ASG | PYGMDV | SEQ ID | SEQ ID | SEQ ID |
| | NO: 106 | SEQ ID NO: 112 | SEQ ID NO: 113 | NO: 109 | NO: 114 | NO: 115 |
| 29 | DYAMA | FIRGWAYGQSAQYGKS | EQRGANGRYGGDGFGY | RASHFIANYVN | ESSTLNR | QQSHSPPVS |
| | SEQ ID | ASG | SYGMDV | SEQ ID | SEQ ID | SEQ ID |
| | NO: 106 | SEQ ID NO: 116 | SEQ ID NO: 117 | NO: 109 | NO: 118 | NO: 119 |

TABLE AZ
CDRs (Chothia) for Anti-HIV gp120 V3 Glycan-Binding antibodies

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 30 | GASISD | YVHKSGDTN | TLHGRRIYGIVAFNEWFTYFYM | GEKSLGSRAVQ | NNQDRPS | HIWDSRVPTKWV |
| | SEQ ID | SEQ ID | DV | SEQ ID NO: 10 | SEQ ID | SEQ ID NO: 12 |
| | NO: 120 | NO: 121 | SEQ ID NO: 9 | | NO: 11 | |
| 31 | GDSMNN | YISDRESAT | ARRGQRIYGVVSFGEFFYYYSM | GRQALGSRAVQ | NNQDRPS | HMWDSRSGFSWS |
| | SEQ ID | SEQ ID | DV | SEQ ID NO: 17 | SEQ ID | SEQ ID NO: 18 |
| | NO: 122 | NO: 123 | SEQ ID NO: 16 | | NO: 11 | |
| 32 | GGSISN | YISDRETTT | ARRGQRIYGVVSFGEFFYYYYM | GRQALGSRAVQ | NNQDRPS | HMWDSRSGFSWS |
| | SEQ ID | SEQ ID | DV | SEQ ID NO: 17 | SEQ ID | SEQ ID NO: 18 |
| | NO: 124 | NO: 125 | SEQ ID NO: 20 | | NO: 11 | |
| 33 | NGSVSG | YFSDTDRSE | AQQGKRIYGIVSFGEFFYYYYM | GERSRGSRAVQ | NNQDRPA | HYWDSRSPISWI |
| | SEQ ID | SEQ ID | DA | SEQ ID NO: 24 | SEQ ID | SEQ ID NO: 26 |
| | NO: 126 | NO: 127 | SEQ ID NO: 23 | | NO: 25 | |
| 34 | NGSVSG | YESDTDRSE | AQQGKRIYGIVSFGELFYYYYM | GERSRGSRAVQ | NNQDRPA | HYWDSRSPISWI |
| | SEQ ID | SEQ ID | DA | SEQ ID NO: 24 | SEQ ID | SEQ ID NO: 26 |
| | NO: 126 | NO: 127 | SEQ ID NO: 27 | | NO: 25 | |
| 35 | GTLVRD | YVHDSGDTN | TKHGRRIYGVVAFKEWFTYFYM | GEESLGSRSVI | NNNDRPS | HIWDSRRPTNWV |
| | SEQ ID | SEQ ID | DV | SEQ ID NO: 31 | SEQ ID | SEQ ID NO: 33 |
| | NO: 128 | NO: 129 | SEQ ID NO: 30 | | NO: 32 | |
| 36 | GASIND | YVHHSGDTN | ALHGKRIYGIVALGELFTYFYM | GKESIGSRAVQ | NNQDRPA | HIYDARGGTNWV |
| | SEQ ID | SEQ ID | DV | SEQ ID NO: 37 | SEQ ID | SEQ ID NO: 38 |
| | NO: 130 | NO: 131 | SEQ ID NO: 36 | | NO: 25 | |
| 37 | GESTGACT | SLSHCQSFW | FDGEVLVYNHWPKPAWVDL | NGTATNFVS | GVDKREP | GSLVGNWDVI |
| | SEQ ID | GSGWTF | SEQ ID NO: 41 | SEQ ID NO: 42 | SEQ ID | SEQ ID NO: 44 |
| | NO: 132 | SEQ ID | | | NO: 43 | |
| | | NO: 133 | | | | |
| 38 | GDSTAACD | GLSHCAGYY | FDGEVLVYHDWPKPAWVDL | TGTSNRFVS | GVNKRPS | SSLVGNWDVI |
| | SEQ ID | NTGWTY | SEQ ID NO: 47 | SEQ ID NO: 48 | SEQ ID | SEQ ID NO: 50 |
| | NO: 134 | SEQ ID | | | NO: 49 | |
| | | NO: 135 | | | | |
| 39 | GDSTAACD | SLSHCAGYY | FGGDVLVYHDWPKPAWVDL | TGNINNFVS | GVNKRPS | GSLAGNWDVV |
| | SEQ ID | NSGWTY | SEQ ID NO: 52 | SEQ ID NO: 53 | SEQ ID | SEQ ID NO: 54 |
| | NO: 134 | SEQ ID | | | NO: 49 | |
| | | NO: 136 | | | | |
| 40 | GDSTAACN | SLSHCASYW | FGGEVLRYTDWPKPAWVDL | TGTSNNFVS | DVNKRPS | GSLVGNWDVI |
| | SEQ ID | NRGWTYHNP | SEQ ID NO: 57 | SEQ ID NO: 58 | SEQ ID | SEQ ID NO: 44 |
| | NO: 137 | SLKS | | | NO: 59 | |
| | | SEQ ID | | | | |
| | | NO: 56 | | | | |
| 41 | GDSTAGCD | GLSHCAGYY | EDGEVLVYNDWPKPAWVDL | TGTSNNFVS | GVNKRPS | GSLVGNWDVI |
| | SEQ ID | NTGWTY | SEQ ID NO: 61 | SEQ ID NO: 58 | SEQ ID | SEQ ID NO: 44 |
| | NO: 138 | SEQ ID | | | NO: 49 | |
| | | NO: 135 | | | | |
| 42 | GESINTGH | HIHYTTAVE | SGGDILYYYEWQKPHWESP | NGTSSDIGGWNE | EVNKRPS | SSLFGRWDVV |
| | SEQ ID | SEQ ID | SEQ ID NO: 64 | VS | SEQ ID | SEQ ID NO: 67 |
| | NO: 139 | NO: 140 | | SEQ ID NO: 65 | NO: 66 | |
| 43 | GGSMRGTDWG | SIHWRGRITH | HKYHDIFRVVPVAGWEDP | RASQNVKNNLA | DASSRAG | QQYEEWPRT |
| | END | SEQ ID | SEQ ID NO: 70 | SEQ ID NO: 71 | SEQ ID | SEQ ID NO: 73 |
| | SEQ ID | NO: 142 | | | NO: 72 | |
| | NO: 141 | | | | | |
| 44 | GGSIRGGEWG | SIHWRGTTH | HKYHDIVMVVPIAGWEDP | RASQSVKNNLA | DTSSRAS | QQYEEWPRT |
| | DSD | SEQ ID | SEQ ID NO: 76 | SEQ ID NO: 77 | SEQ ID | SEQ ID NO: 73 |
| | SEQ ID | NO: 144 | | | NO: 78 | |
| | NO: 143 | | | | | |
| 45 | GDSIRGGEWG | SIHWRGTTH | HRHHDVEMLVPIAGWEDV | RASQNINKNLA | ETYSKIA | QQYEEWPRT |
| | DKD | SEQ ID | SEQ ID NO: 81 | SEQ ID NO: 82 | SEQ ID | SEQ ID NO: 73 |
| | SEQ ID | NO: 144 | | | NO: 83 | |
| | NO: 145 | | | | | |
| 46 | QDSRPSDH | HYNGA | NAIRIYGVVALGEWFHYGMDV | SGAPLTSRFTY | RSSQRSS | QSSDTSDSYKM |
| | SEQ ID | SEQ ID | SEQ ID NO: 86 | SEQ ID NO: 87 | SEQ ID | SEQ ID NO: 89 |
| | NO: 146 | NO: 147 | | | NO: 88 | |
| 47 | NDSRPSDH | HYNGA | NAIRIYGVVALGEWFHYGMDV | SGAPLTSRFTY | RSSQRSS | QSSDTSDSYKM |
| | SEQ ID | SEQ ID | SEQ ID NO: 86 | SEQ ID NO: 87 | SEQ ID | SEQ ID NO: 89 |
| | NO: 148 | NO: 147 | | | NO: 88 | |
| 48 | GDSRPSDH | HYNGD | NVIRVEGVISLGEWEHYGMDV | SGPPLASRYTY | RDRQFPS | QSSDTSDSYKM |
| | SEQ ID | SEQ ID | SEQ ID NO: 91 | SEQ ID NO: 92 | SEQ ID | SEQ ID NO: 89 |
| | NO: 149 | NO: 150 | | | NO: 93 | |
| 49 | NDSRPSDH | HYNGA | NAIRIYGVVALGEWFHYGMDV | SGAALTSRFTY | RTSQRSS | QSSDTSDSYKM |
| | SEQ ID | SEQ ID | SEQ ID NO: 86 | SEQ ID NO: 95 | SEQ ID | SEQ ID NO: 89 |
| | NO: 148 | NO: 147 | | | NO: 96 | |
| 50 | GDSRPSDH | HYGGD | NVIRVFGVIALGEWEHYGMDV | SGPPLASRYCY | RDRQFSS | QSSDINDSYKM |
| | SEQ ID | SEQ ID | SEQ ID NO: 98 | SEQ ID NO: 99 | SEQ ID | SEQ ID |
| | NO: 149 | NO: 151 | | | NO: 100 | NO: 101 |
| 51 | GDSRPSDH | HYGGD | NVIRVEGVIALGEWFHYGMDV | SGPPLASRYCY | RDRQFSS | QSSDTSDSFKM |
| | SEQ ID | SEQ ID | SEQ ID NO: 98 | SEQ ID NO: 99 | SEQ ID | SEQ ID |
| | NO: 149 | NO: 151 | | | NO: 100 | NO: 102 |
| 52 | GDSRPSDH | HYGGD | NVIRVFGVIALGEWEHYGMDV | SGPPLATRYCY | RDRQFSS | QSSDTSDSYKM |
| | SEQ ID | SEQ ID | SEQ ID NO: 98 | SEQ ID | SEQ ID | SEQ ID NO: 89 |
| | NO: 149 | NO: 151 | | NO: 103 | NO: 100 | |
| 53 | GDSRPSDH | HYGGD | NVIRVEGVIALGEWFHYGMDV | SGPPLASRYCY | RDRQFSS | QSSDNSDSFKM |
| | SEQ ID | SEQ ID | SEQ ID NO: 98 | SEQ ID NO: 99 | SEQ ID | SEQ ID |
| | NO: 149 | NO: 151 | | | NO: 100 | NO: 105 |
| 54 | GFYFPDY | RGWAYGGS | EQRNKDYRYGQEGFGYSYGMDV | RASHFIANYVN | ESSTLQR | QQSHSPPVT |
| | SEQ ID | SEQ ID | SEQ ID NO: 108 | SEQ ID | SEQ ID | SEQ ID |
| | NO: 152 | NO: 153 | | NO: 109 | NO: 110 | NO: 111 |
| 55 | DFYFPDY | RGWAYGQA | EQRGGDGRYSGDGFGYPYGMDV | RASHFIANYVN | QSWTLNR | QQSHSPPLS |
| | SEQ ID | SEQ ID | SEQ ID NO: 113 | SEQ ID | SEQ ID | SEQ ID |
| | NO: 154 | NO: 155 | | NO: 109 | NO: 114 | NO: 115 |
| 56 | DFYFPDY | RGWAYGQS | EQRGANGRYGGDGFGYSYGMDV | RASHFIANYVN | ESSTLNR | QQSHSPPVS |
| | SEQ ID | SEQ ID | SEQ ID NO: 117 | SEQ ID | SEQ ID | SEQ ID |
| | NO: 154 | NO: 156 | | NO: 109 | NO: 118 | NO: 119 |

TABLE A3
CDRs (IMGT) for Anti-HIV gp120 V3 Glycan-Binding antibodies

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 57 | GASISDSY | VHKSGDT | ARTLHGRRIYGIVAFNEWETYFYMDV | SLGSRA | NNQ | HIWDSRVPTKWV |
| | SEQ ID | SEQ ID | SEQ ID NO: 159 | SEQ ID | SEQ ID | SEQ ID NO: 12 |
| | NO: 157 | NO: 158 | | NO: 160 | NO: 161 | |
| 58 | GDSMNNYY | ISDRESA | ATARRGQRIYGVVSFGEFFYYYSMDV | ALGSRA | NNQ | HMWDSRSGFSWS |
| | SEQ ID | SEQ ID | SEQ ID NO: 164 | SEQ ID | SEQ ID | SEQ ID NO: 18 |
| | NO: 162 | NO: 163 | | NO: 165 | NO: 161 | |
| 59 | GDSMNNYY | ISDRESA | ARARRGQRIYGVVSFGEFFYYYSMDV | ALGSRA | NNQ | HMWDSRSGFSWS |
| | SEQ ID | SEQ ID | SEQ ID NO: 166 | SEQ ID | SEQ ID | SEQ ID NO: 18 |
| | NO: 162 | NO: 163 | | NO: 165 | NO: 161 | |
| 60 | GGSISNYY | ISDRETT | ATARRGQRIYGVVSFGEFFYYYYMDV | ALGSRA | NNO | HMWDSRSGFSWS |
| | SEQ ID | SEQ ID | SEQ ID NO: 169 | SEQ ID | SEQ ID | SEQ ID NO: 18 |
| | NO: 167 | NO: 168 | | NO: 165 | NO: 161 | |
| 61 | NGSVSGRF | FSDTDRS | ARAQQGKRIYGIVSFGELFYYYYMDA | SRGSRA | NNQ | HYWDSRSPISWI |
| | SEQ ID | SEQ ID | SEQ ID NO: 172 | SEQ ID | SEQ ID | SEQ ID NO: 26 |
| | NO: 170 | NO: 171 | | NO: 173 | NO: 161 | |
| 62 | NGSVSGRF | ESDTDRS | ARAQQGKRIYGIVSFGEFFYYYYMDA | SRGSRA | NNQ | HYWDSRSPISWI |
| | SEQ ID | SEQ ID | SEQ ID NO: 174 | SEQ ID | SEQ ID | SEQ ID NO: 26 |
| | NO: 170 | NO: 171 | | NO: 173 | NO: 161 | |
| 63 | GTLVRDNY | VHDSGDT | ATTKHGRRIYGVVAFKEWFTYFYMDV | SIGSRA | NNQ | HIYDARGGTNWV |
| | SEQ ID | SEQ ID | SEQ ID NO: 177 | SEQ ID | SEQ ID | SEQ ID NO: 38 |
| | NO: 175 | NO: 176 | | NO: 178 | NO: 161 | |
| 64 | GASINDAY | VHHSGDT | ARALHGKRIYGIVALGELFTYFYMDV | SLGSRS | NNN | HIWDSRRPTNWV |
| | SEQ ID | SEQ ID | SEQ ID NO: 181 | SEQ ID | SEQ ID | SEQ ID NO: 33 |
| | NO: 179 | NO: 180 | | NO: 182 | NO: 183 | |
| 65 | GESTGACTYF | LSHCQSFWG | ARFDGEVLVYNHWPKPAWVDL | ATNF | GVD | GSLVGNWDVI |
| | SEQ ID | SGWT | SEQ ID NO: 186 | SEQ ID | SEQ ID | SEQ ID NO: 44 |
| | NO: 184 | SEQ ID | | NO: 187 | NO: 188 | |
| | | NO: 185 | | | | |
| 66 | GDSTAACDYF | ISHCAGYYN | ARFDGEVLVYHDWPKPAWVDL | SNRF | GVN | SSLVGNWDVI |
| | SEQ ID | TGWT | SEQ ID NO: 191 | SEQ ID | SEQ ID | SEQ ID NO: 50 |
| | NO: 189 | SEQ ID | | NO: 192 | NO: 193 | |
| | | NO: 190 | | | | |
| 67 | GDSTAACDYF | ISHCAGYYN | ARFGGDVLVYHDWPKPAWVDL | INNF | GVN | GSLAGNWDVV |
| | SEQ ID | SGWT | SEQ ID NO: 195 | SEQ ID | SEQ ID | SEQ ID NO: 54 |
| | NO: 189 | SEQ ID | | NO: 196 | NO: 193 | |
| | | NO: 194 | | | | |
| 68 | GDSTAANSF | ISHCASYWN | ARFGGEVLRYTDWPKPAWVDL | SNNF | DVN | GSLVGNWDVI |
| | SEQ ID | RGWT | SEQ ID NO: 199 | SEQ ID | SEQ ID | SEQ ID NO: 44 |
| | NO: 197 | SEQ ID | | NO: 200 | NO: 201 | |
| | | NO: 198 | | | | |
| 69 | GDSTAGCDYF | LSHCAGYYN | ARFDGEVLVYNDWPKPAWVDL | SNNF | GVN | GSLVGNWDVI |
| | SEQ ID | TGWT | SEQ ID NO: 204 | SEQ ID | SEQ ID | SEQ ID NO: 44 |
| | NO: 202 | SEQ ID | | NO: 200 | NO: 193 | |
| | | NO: 203 | | | | |
| 70 | GESINTGHYY | IHYTTAV | VRSGGDILYYYEWQKPHWFSP | SSDIGGWNF | EVN | SSLFGRWDVV |
| | SEQ ID | SEQ ID | SEQ ID NO: 207 | SEQ ID | SEQ ID | SEO ID NO: 67 |
| | NO: 205 | NO: 206 | | NO: 208 | NO: 209 | |
| 71 | GGSMRGTDWG | IHWRGRTT | ARHKYHDIFRVVPVAGWEDP | QNVKNN | DAS | QQYEEWPRT |
| | ENDFH | SEQ ID | SEQ ID NO: 212 | SEQ ID | SEQ ID | SEQ ID NO: 73 |
| | SEQ ID | NO: 211 | | NO: 213 | NO: 214 | |
| | NO: 210 | | | | | |
| 72 | GGSIRGGEWG | IHWRGTT | VKHKYHDIVMVVPIAGWFDP | QSVKNN | DTS | QQYEEWPRT |
| | DSDYH | SEQ ID | SEQ ID NO: 217 | SEQ ID | SEQ ID | SEQ ID NO: 73 |
| | SEQ ID | NO: 216 | | NO: 218 | NO: 219 | |
| | NO: 215 | | | | | |
| 73 | GDSIRGGEWG | IHWRGTT | ARHRHHDVFMLVPIAGWFDV | QNINKN | ETY | QQYEEWPRT |
| | DKDYH | SEQ ID | SEQ ID NO: 221 | SEQ ID | SEQ ID | SEQ ID NO: 73 |
| | SEQ ID | NO: 216 | | NO: 222 | NO: 223 | |
| | NO: 220 | | | | | |
| 74 | QDSRPSDHS | IHYNGAT | NAIRIYGVVALGEWFHYGMDV | PLTSRF | RSS | QSSDTSDSYKM |
| | SEQ ID | SEQ ID | SEQ ID NO: 86 | SEQ ID | SEQ ID | SEQ ID NO: 89 |
| | NO: 224 | NO: 225 | | NO: 226 | NO: 227 | |
| 75 | NDSRPSDHS | IHYNGAT | NAIRIYGVVALGEWFHYGMDV | PLTSRF | RSS | QSSDTSDSYKM |
| | SEQ ID | SEQ ID | SEQ ID NO: 86 | SEQ ID | SEQ ID | SEQ ID NO: 89 |
| | NO: 228 | NO: 225 | | NO: 226 | NO: 227 | |
| 76 | GDSRPSDHS | VHYNGDN | NVIRVFGVISLGEWFHYGMDV | PLASRY | RDR | QSSDTSDSYKM |
| | SEQ ID | SEQ ID | SEQ ID NO: 91 | SEQ ID | SEQ ID | SEQ ID NO: 89 |
| | NO: 229 | NO: 230 | | NO: 231 | NO: 232 | |
| 77 | GDSRPSDHS | VHYNGDT | NVIRVFGVISLGEWFHYGMDV | PLASRY | RDR | QSSDTSDSYKM |
| | SEQ ID | SEQ ID | SEQ ID NO: 91 | SEQ ID | SEQ ID | SEQ ID NO: 89 |
| | NO: 229 | NO: 233 | | NO: 231 | NO: 232 | |
| 78 | NDSRPSDHS | IHYNGAT | NAIRIYGVVALGEWFHYGMDV | ALTSRF | RTS | QSSDTSDSYKM |
| | SEQ ID | SEQ ID | SEQ ID NO: 86 | SEQ ID | SEQ ID | SEQ ID NO: 89 |
| | NO: 228 | NO: 225 | | NO: 234 | NO: 235 | |
| 79 | GDSRPSDHS | IHYGGDI | NVIRVFGVIALGEWFHYGMDV | PLASRY | RDR | QSSDINDSYKM |
| | SEQ ID | SEQ ID | SEQ ID NO: 98 | SEQ ID | SEQ ID | SEQ ID |
| | NO: 229 | NO: 236 | | NO: 231 | NO: 232 | NO: 101 |
| 80 | GDSRPSDHS | IHYGGDI | NVIRVFGVIALGEWFHYGMDV | PLASRY | RDR | QSSDTSDSFKM |
| | SEQ ID | SEQ ID | SEQ ID NO: 98 | SEQ ID | SEQ ID | SEQ ID |
| | NO: 229 | NO: 236 | | NO: 231 | NO: 232 | NO: 102 |
| 81 | GDSRPSDHS | IHYGGDI | NVIRVFGVIALGEWFHYGMDV | PLATRY | RDR | QSSDTSDSYKM |
| | SEQ ID | SEQ ID | SEQ ID NO: 98 | SEQ ID | SEQ ID | SEQ ID NO: 89 |
| | NO: 229 | NO: 236 | | NO: 237 | NO: 232 | |
| 82 | GDSRPSDHS | THYNGDK | NVIRVFGVISLGEWFHYGMDV | PLASRY | RDR | QSSDTSDSYKM |
| | SEQ ID | SEQ ID | SEQ ID NO: 91 | SEQ ID | SEQ ID | SEQ ID NO: 89 |
| | NO: 229 | NO: 238 | | NO: 231 | NO: 232 | |
| 83 | GDSRPSDHS | IHYGGDI | NVIRVFGVIALGEWFHYGMDV | PLASRY | RDR | QSSDNSDSFKM |
| | SEQ ID | SEQ ID | SEQ ID NO: 98 | SEQ ID | SEQ ID | SEQ ID |
| | NO: 229 | NO: 236 | | NO: 231 | NO: 232 | NO: 105 |
| 84 | GFYFPDYA | MRGWAYGGSA | FQRNKDYRYGQEGFGYSYGMDV | HFIANY | ESS | QQSHSPPVT |
| | SEQ ID | SEQ ID | SEQ ID NO: 108 | SEQ ID | SEQ ID | SEQ ID |
| | NO: 239 | NO: 240 | | NO: 241 | NO: 242 | NO: 111 |
| 85 | DFYFPDYA | IRGWAYGQAA | EQRGGDGRYSGDGFGYPYGMDV | HEIANY | QSW | QQSHSPPLS |
| | SEQ ID | SEQ ID | SEQ ID NO: 113 | SEQ ID | SEQ ID | SEQ ID |
| | NO: 243 | NO: 244 | | NO: 241 | NO: 245 | NO: 115 |
| 86 | DFYFPDYA | IRGWAYGQSA | KQRGANGRYGGDGFGYSYGMDV | AFIANY | ESS | QQSHSPPVS |
| | SEQ ID | SEQ ID | SEQ ID NO: 117 | SEQ ID | SEQ ID | SEQ ID |
| | NO: 243 | NO: 246 | | NO: 241 | NO: 242 | NO: 119 |

TABLE A4
CDRs (Honegger) for Anti-HIV gp120 V3 Glycan-Binding antibodies

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 87 | VSGASISDSY | VHKSGDTNYSPSLKSR | TLHGRRIYGIVAFN | EKSLGSRA | NNQDRPSGIPER | WDSRVPTKW |
| | SEQ ID NO: 247 | SEQ ID NO: 248 | EWFTYFYMD | SEQ ID | SEQ ID | SEQ ID |
| | | | SEQ ID NO: 249 | NO: 250 | NO: 251 | NO: 252 |
| 88 | VSGASISDSY | VHKSGDTNYNPSLKSR | TLHGRRIYGIVAFN | EKSLGSRA | NNQDRPSGIPER | WDSRVPTKW |
| | SEQ ID NO: 247 | SEQ ID NO: 253 | EWFTYFYND | SEQ ID | SEQ ID | SEQ ID |
| | | | SEQ ID NO: 249 | NO: 250 | NO: 251 | NO: 252 |
| 89 | VSGDSMNNYY | ISDRESATYNPSLNSR | ARRGQRIYGVVSFG | RQALGSRA | NNODRPSGIPER | WDSRSGFSW |
| | SEQ ID NO: 254 | SEQ ID NO: 255 | EFFYYYSMD | SEQ ID | SEQ ID | SEQ ID |
| | | | SEQ ID NO: 256 | NO: 257 | NO: 251 | NO: 258 |
| 90 | VSGGSISNYY | ISDRETTTYNPSLNSR | ARRGQRIYGVVSFG | RQALGSRA | NNQDRPSGIPER | WDSRSGFSW |
| | SEQ ID NO: 259 | SEQ ID NO: 260 | EFFYYYYMD | SEQ ID | SEQ ID | SEQ ID |
| | | | SEQ ID NO: 261 | NO: 257 | NO: 251 | NO: 258 |
| 91 | VSNGSVSGRF | FSDTDRSEYNPSLRSR | AQQGKRIYGIVSFG | ERSRGSRA | NNQDRPAGVSER | WDSRSPISW |
| | SEQ ID NO: 262 | SEQ ID NO: 263 | ELFYYYYMD | SEQ ID | SEQ ID | SEQ ID |
| | | | SEQ ID NO: 264 | NO: 265 | NO: 266 | NO: 267 |
| 92 | VSNGSVSGRF | FSDTDRSEYNPSLRSR | AQQGKRIYGIVSFG | ERSRGSRA | NNQDRPAGVSER | WDSRSPISW |
| | SEQ ID NO: 262 | SEQ ID NO: 263 | EFFYYYYMD | SEQ ID | SEQ ID | SEQ ID |
| | | | SEQ ID NO: 268 | NO: 265 | NO: 266 | NO: 267 |
| 93 | VSGASINDAY | VHHSGDTNYNPSLKRR | ALHGKRIYGIVALG | KESIGSRA | NNQDRPAGVPER | YDARGGTNW |
| | SEQ ID NO: 269 | SEQ ID NO: 270 | ELFTYFYMD | SEQ ID | SEQ ID | SEQ ID |
| | | | SEQ ID NO: 271 | NO: 272 | NO: 273 | NO: 274 |
| 94 | VSGTLVRDNY | VHDSGDTNYNPSLKSR | TKHGRRIYGVVAFK | EESLGSRS | NNNDRPSGIPDR | WDSRRPTNW |
| | SEQ ID NO: 275 | SEQ ID NO: 276 | EWFTYFYMD | SEQ ID | SEQ ID | SEQ ID |
| | | | SEQ ID NO: 277 | NO: 278 | NO: 279 | NO: 280 |
| 95 | VSGESTGACTYF | LSHCQSFWGSGWTFHNP | FDGEVLVYNHWPKP | GTATNF | GVDKRPPGVPDR | LVGNWDV |
| | SEQ ID NO: 281 | SLKSR | AWVD | SEQ ID | SEQ ID | SEQ ID |
| | | SEQ ID NO: 282 | SEQ ID NO: 283 | NO: 284 | NO: 285 | NO: 286 |
| 96 | VSGDSTAACDYF | LSHCAGYYNTGWTYHNP | FDGEVLVYHDWPKP | GTSNRF | GVNKRPSGVPDR | LVGNWDV |
| | SEQ ID NO: 287 | SLKSR | AWVD | SEQ ID | SEQ ID | SEQ ID |
| | | SEQ ID NO: 288 | SEQ ID NO: 289 | NO: 290 | NO: 291 | NO: 286 |
| 97 | VSGDSTAACDYF | LSHCAGYYNSGWTYHNP | FGGDVLVYHDWPKP | GNINNF | GVNKRPSGVPDR | LAGNWDV |
| | SEQ ID NO: 287 | SLKSR | AWVD | SEQ ID | SEQ ID | SEQ ID |
| | | SEQ ID NO: 292 | SEQ ID NO: 293 | NO: 294 | NO: 291 | NO: 295 |
| 98 | VSGDSTAAGNSF | ISHCASYWNRGWTYHNP | FGGEVLRYTDWPKP | GTSNNF | DVNKRPSGVPDR | LVGNWDV |
| | SEQ ID NO: 296 | SLKSR | AWVD | SEQ ID | SEQ ID | SEQ ID |
| | | SEQ ID NO: 297 | SEQ ID NO: 298 | NO: 299 | NO: 300 | NO: 286 |
| 99 | VSGDSTAGCDYF | LSHCAGYYNTGWTYHNP | FDGEVLVYNDWPKP | GTSNNF | GVNKRPSGVPDR | LVGNWDV |
| | SEQ ID NO: 301 | SLKSR | AWVD | SEQ ID | SEQ ID | SEQ ID |
| | | SEQ ID NO: 288 | SEQ ID NO: 302 | NO: 299 | NO: 291 | NO: 286 |
| 100 | VSGESINTGHYY | IHYTTAVLHNPSLKSR | SGGDILYYYEWQKP | GTSSDIGGWNF | EVNKRPSGVPGR | LFGRWDV |
| | SEQ ID NO: 303 | SEQ ID NO: 304 | HWFS | SEQ ID | SEQ ID | SEQ ID |
| | | | SEQ ID NO: 305 | NO: 306 | NO: 307 | NO: 308 |
| 101 | VSGGSMRGTDWGE | IHWRGRTTHYKTSFRSR | HKYHDIFRVVPVAG | ASQNVKNN | DASSRAGGIPDR | YEEWPR |
| | NDFH | SEQ ID NO: 310 | WED | SEQ ID | SEO ID | SEQ ID |
| | SEQ ID NO: 309 | | SEQ ID NO: 311 | NO: 312 | NO: 313 | NO: 314 |
| 102 | ASGGSIRGGEWGD | IHWRGTTHYNAPFRGR | HKYHDIVMVVPIAG | ASQSVKMN | DTSSRASGIPAR | YEEWPR |
| | SDYH | SEQ ID NO: 316 | WFD | SEQ ID | SEQ ID | SEQ ID |
| | SEQ ID NO: 315 | | SEQ ID NO: 317 | NO: 318 | NO: 319 | NO: 314 |
| 103 | VSGDSIRGGEWGD | IHWRGTTHYKESLRRR | HRHRDVFMLVPIAG | ASQNINKN | ETYSKIAAFPAR | YEEWPR |
| | KDYH | SEQ ID NO: 321 | WFD | SEQ ID | SEQ ID | SEQ ID |
| | SEQ ID NO: 320 | | SEQ ID NO: 322 | NO: 323 | NO: 324 | NO: 314 |
| 104 | VSQDSRPSDHS | IHYNGATTYNPSLRSR | NAIRIYGVVALGEW | GAPLTSRF | RSSQRSSGWSGR | SDTSDSYK |
| | SEQ ID NO: 325 | SEQ ID NO: 326 | FRYGMD | SEQ ID | SEQ ID | SEQ ID |
| | | | SEQ ID NO: 327 | NO: 328 | NO: 329 | NO: 330 |
| 105 | VSNDSRPSDHS | IHYNGATTYNPSLRSR | NAIRIYGVVALGEW | GAPLTSRF | RSSQRSSGWSGR | SDTSDSYK |
| | SEQ ID NO: 331 | SEQ ID NO: 326 | FHYGMD | SEQ ID | SEQ ID | SEQ ID |
| | | | SEQ ID NO: 327 | NO: 328 | NO: 329 | NO: 330 |
| 106 | VFGDSRPSDHS | VHYNGDNTYNPSLRGR | NVIRVFGVISLGEW | GPPLASRY | RDRQFPSGVSGR | SDTSDSYK |
| | SEQ ID NO: 332 | SEQ ID NO: 333 | FHYGMD | SEQ ID | SEQ ID | SEQ ID |
| | | | SEQ ID NO: 334 | NO: 335 | NO: 336 | NO: 330 |
| 107 | VFGDSRPSDHS | VHYNGDTTYNPSLRGR | NVIRVFGVISLGEW | GPPLASRY | RDRQEPSGVSGR | SDTSDSYK |
| | SEQ ID NO: 332 | SEQ ID NO: 337 | FRYGMD | SEQ ID | SEQ ID | SEQ ID |
| | | | SEQ ID NO: 334 | NO: 335 | NO: 336 | NO: 330 |
| 108 | VSNDSRPSDHS | IHYNGATTYNPSERSR | NAIRIYGVVALGEW | GAALTSRF | RTSQRSSGWSGR | SDTSDSYK |
| | SEQ ID NO: 331 | SEQ ID NO: 326 | FHYGMD | SEQ ID | SEQ ID | SEQ ID |
| | | | SEQ ID NO: 327 | NO: 338 | NO: 339 | NO: 330 |
| 109 | ISGDSRPSDHS | IHYGGDITYNPSLRSR | NVIRVFGVIALGEW | GPPLASRY | RDRQFSSGMSGR | SDINDSYK |
| | SEQ ID NO: 340 | SEQ ID NO: 341 | FHYGMD | SEQ ID | SEQ ID | SEQ ID |
| | | | SEQ ID NO: 342 | NO: 335 | NO: 343 | NO: 344 |
| 110 | ISGDSRPSDHS | IHYGGDITYNPSLRSR | NVIRVFGVIALGEW | GPPLASRY | RDRQFSSGISGR | SDTSDSFK |
| | SEQ ID NO: 340 | SEQ ID NO: 341 | FHYGMD | SEQ ID | SEQ ID | SEQ ID |
| | | | SEQ ID NO: 342 | NO: 335 | NO: 345 | NO: 346 |
| 111 | ISGDSRPSDHS | IHYGGDITYNPSLRSR | NVIRVEGVIALGEW | GPPLATRY | RDRQFSSGVSGR | SDTSDSYK |
| | SEQ ID NO: 340 | SEQ ID NO: 341 | FHYGMD | SEQ ID | SEQ ID | SEQ ID |
| | | | SEQ ID NO: 342 | NO: 347 | NO: 348 | NO: 330 |
| 112 | VFGDSRPSDHS | IHYNGDKTYNPSLRGR | NVIRVEGVISLGEW | GPPLASRY | RDRQFPSGVSGR | SDTSDSYK |
| | SEQ ID NO: 332 | SEQ ID NO: 349 | FHYGMD | SEQ ID | SEQ ID | SEQ ID |
| | | | SEQ ID NO: 334 | NO: 335 | NO: 336 | NO: 330 |
| 113 | ISGDSRPSDHS | IHYGGDITYNPSLRSR | NVIRVFGVIALGEW | GPPLASRY | RDRQFSSGISGR | SDNSDSFK |
| | SEQ ID NO: 340 | SEQ ID NO: 341 | WHYGMD | SEQ ID | SEQ ID | SEQ ID |
| | | | SEQ ID NO: 342 | NO: 335 | NO: 345 | NO: 350 |
| 114 | ASGFYFPDYA | MRGWAYGGSAQFAAFAV | EQRNKDYRYGQEGF | ASHFIANY | ESSTLQRGVPSR | SHSPPV |
| | SEQ ID NO: 351 | GK | GYSYGMD | SEQ ID | SEQ ID | SEQ ID |
| | | SEQ ID NO: 352 | SEQ ID NO: 353 | NO: 354 | NO: 355 | NO: 356 |
| 115 | AFDFYFPDYA | IRGWAYGQAAQYGKSAS | EQRGGDGRYSGDGF | ASHFIANY | QSWTLNRGIPSR | SHSPPL |
| | SEQ ID NO: 357 | GR | GYPYGMD | SEQ ID | SEQ ID | SEQ ID |
| | | SEQ ID NO: 358 | SEQ ID NO: 359 | NO: 354 | NO: 360 | NO: 361 |
| 116 | AFDFYEPDYA | IRGWAYGQSAQYGKSAS | EQRGANGRYGGDGF | ASHFIANY | ESSTLNRGVPSR | SHSPPV |
| | SEQ ID NO: 357 | GR | GYSYGMD | SEQ ID | SEQ ID | SEQ ID |
| | | SEQ ID NO: 362 | SEQ ID NO: 363 | NO: 354 | NO: 364 | NO: 356 |

TABLE B
VH/VL for Anti-HIV gp120 V3 Glycan-Binding antibodies

| | SEO | | SEQ | |
|---|---|---|---|---|
| Ab | ID | | ID | |
| Name | NO | VH | NO | VL |
| 117 | 365 | QMQLQESGPGLVKPSETLSLTCSVSGASISDSYWSWI | 366 | SDISVAPGETARISCGEKSLGSRAVQWYQHRA |
| | | RRSPGKGLEWIGYVHKSGDTNYSPSLKSRVNLSLDTS | | GQAPSLIIYNNQDRPSGIPERFSGSPDSPEGT |
| | | KNQVSLSLVAATAADSGKYYCARTLHGRRIYGIVAFN | | TATLTITSVEAGDEADYYCHIWDSRVPTKWVE |
| | | EWFTYFYMDVWENGTQVTVSS | | GGGTTLTVL |
| 118 | 367 | QMQLQESGPGLVKPSETLSLTCSVSGASISDSYWSWI | 368 | SDISVAPGETARISCGEKSLGSRAVQWYQHRA |
| | | RRSPGKGLEWIGYVHKSGDTNYNPSLKSRVHLSLDTS | | GQAPSLIIYNNQDRPSGIPERFSGSPDFRPGT |
| | | KNQVSLSLTGVTAADSGKYYCARTLHGRRIYGIVAFN | | TATLTITSVEAGDEADYYCHIWDSRVPTKWVE |
| | | EWFTYFYNDVWGIGTQVTVSS | | GGGTTLTVE |
| 119 | 369 | QMQLQESGPGLVKPSETLSLTCSVSGASISDSYWSWI | 370 | SDISVAPGETARISCGEKSLGSRAVQWYQHRA |
| | | RRSPGKGLEWIGYVHKSGDTNYNPSLKSRVALSLDTS | | GQAPSLIIYNNQDRPSGIPERFSGSPDSRPGT |
| | | KNQVSLSLTGVTAADSGKYYCARTLHGRRIYGIVAFN | | TATLTITSVEAGDEADYYCHIWDSRVPTKWVE |
| | | EWFTYFYMDVWGTGTQVTVSS | | GGGTTLTVL |
| 120 | 371 | QMQLQESGPGLVKPSETLSLTCSVSGASISDSYWSWI | 372 | SDISVAPGETARISCGEKSLGSRAVQWYQQRA |
| | | RQPPGKGLEWIGYVHKSGDTNYSPSLKSRVNLSLDTS | | GQAPSLIIYNNQDRPSGIPERFSGSPDSGEGT |
| | | KNQVSLSLSAATAADSGVYYCARTLAGRRIYGIVAFN | | TATLTITSVEAGDEADYYCHIWDSRVPTKWVE |
| | | EWFTYFYMDVWENGTQVTVSS | | GGGTTLTVE |
| 121 | 373 | QMQLQESGPGLVKPSETLSLTCSVSGASISDSYWSWI | 374 | SDISVAPGETARISCGEKSLGSRAVQWYQHRA |
| | | RRSPGKGLEWIGYVHKSGDTNYNPSLKSRVALSLDTS | | GQAPSLIIYNNQDRPSGIPERFSGSPDSREGT |
| | | KNQVSLSLTGVTAADSGKYYCARTLHGRRIYGIVAEN | | TATLTITSVEAGDEADYYCHIWDSRVPTKWVE |
| | | EWFTYFYMDVWGTGTQVTVSS | | GGGTTLTVL |
| 122 | 375 | QVQLQESGPGLVKPSETLSVTCSVSGDSMNNYYWTWI | 376 | SYVRPLSVALGETARISCGRQALGSRAVQWYQ |
| | | RQSPGKGLEWIGYISDRESATYNPSLNSRVVISRDTS | | HRPCQAPILLIYNNQDRPSGIPERFSGTPDIN |
| | | KNQLSLKLNSVTPADTAVYYCATARRGQRIYGVVSFG | | FGTRATLTISGVEAGDEADYYCHMWDSRSGFS |
| | | EFFYYYSMDVAGKGTTVTVSS | | WSEGGATRLTVL |
| 123 | 377 | QVQLQESGPGLVKPSETLSVTCSVSGDSMNNYYWTWI | 378 | SPVRPLSVALGETARISCGRQALGSRAVQWYQ |
| | | RQSPGKGLEWIGYISDRESATYNPSLNSRVTISRDTS | | HRPGQAPILLIYNNQDRPSGIPERFSGTPDIN |
| | | KNQFSLKLNSVTPADTAVYYCARARRGQRIYGVVSFG | | FGTRATLTISGVEAGDEADYYCHMWDSRSGES |
| | | EFFYYYSMDVWGKGTTVTVSS | | WSEGGATRLTVL |
| 124 | 379 | QVQLQESGPGLVRPSETLSVTCIVSGGSISNYYWTWI | 380 | SYVSPLSVALGETARISQGRQALGSRAVQWYQ |
| | | RQSPGKGLEWIGYISDRETTTYNPSLNSRAVISRDTS | | HKPGQAPILLIYNNQDRPSGIPERFSGTPDIN |
| | | KNQLSLQLRSVTTADTAIYFCATARRGQRIYGVVSEG | | EGTTATLTISGVEVGDEADYYCHMWDSRSGES |
| | | EFFYYYYMDVWGKGTAVTVSS | | WSEGGATRLTV |
| 125 | 381 | QVHLQESGPGLVTPSETLSLTCTVSNGSVSGREWSWI | 382 | SLNPLSLAPGATAKIPCGERSRGSRAVQWYQQ |
| | | RQSPGRGLEWIGYFSDTDRSEYNPSLRSRLTLSVDRS | | KPGQAPTLIIYNNQDRPAGVSERFSGNPDVAI |
| | | KNQLSLRLKSVTAADSATYYCARAQQGKRIYGIVSFG | | GVTATLTISRVEVGDEADYYCHYWDSRSPISW |
| | | EFFYYYYMDAWGKGTPVTVSS | | IFGGGTQLTVL |
| 126 | 383 | QVHLQESGPGLVTPSETLSLTCTVSNGSVSGREWSWI | 384 | SLNPLSLAPGATAKIPCGERSRGSRAVQWYQQ |
| | | RQSPGRGLEWIGYFSDTDRSEYNPSLRSRLTLSVDRS | | KPGQAPTLIIYNNQDRPAGVSERFSGNPDVAI |
| | | KNQLSLKLKSVTAADSATYYCARAQQGKRIYGIVSFG | | GVTATLTISRVEVGDEGDYYCHYWDSRSPISW |
| | | ELFYYYYMDAWGKGTPVTVSS | | IFAGGTQLTVL |
| 127 | 385 | QVHLQESGPGLVKPSETLSLTCNVSGTLVRDNYWSWI | 386 | TEVSVAPGQTARITCGEESLGSRSVIWYQQRP |
| | | RQPLGKQPEWIGYVHDSGDTNYNPSLKSRVHLSLDKS | | GQAPSLIIYNNNDRPSGIPDRFSGSPGSTFGT |
| | | KNIVSLRLTGVTAADSAIYYCATTKAGRRIYGVVAFK | | TATLTITSVEAGDEADYYCHIWDSRRPINWVF |
| | | EWFTYFYMDVWGKGTSVTVSS | | GEGTTLIVL |
| 128 | 387 | QLHLQESGPGLVKPPETLSLTCSVSGASINDAYWSWI | 388 | SSMSVSPGETAKISCGKESIGSRAVQWYQQKP |
| | | RQSPGKRPEWVGYVHHSGDTNYNPSLKRRVIFSLDTA | | GQPPSLIIYNNQDRPAGVPERFSASPDERPGT |
| | | KNEVSLKLVDLTAADSATYFCARALHGKRIYGIVALG | | TATLTITNVDAEDEADYYCHIYDARGGTNWVF |
| | | ELFTYFYMDVWGKGTAVTVSS | | DRGTTLTVL |
| 129 | 389 | QSQLQESGPRLVEASETLSLTCNVSGESTGACTYFWG | 390 | QSALTQPPSASGSPGQSITISCNGTATNFVSW |
| | | WVRQAPGKGLEWIGSLSHCQSFWGSGWTFHNPSLKSR | | YQQFPDKAPKLIIFGVDKRPPGVPDRFSGSRS |
| | | LTISLDTPKNQVFLKLTSLTAADTATYYCARFDGEVL | | GTTASLTVSRLQTDDEAVYYCGSLVGNWDVIF |
| | | VYNHWPKPAWVDLWGRGIPVTVSS | | GGGTTLTVL |
| 130 | 391 | QPQLQESGPGLVEASETLSLTCTVSGDSTAACDYFWG | 392 | QSALTQPPSASGSPGQSISISCTGTSNRFVSW |
| | | WVRQPPGKGLEWIGGLSHCAGYYNTGWTYANPSLKSR | | YQQHPGKAPKLVIYGVNKRPSGVPDRFSGSKS |
| | | LTISLDTPKNQVELKLNSVTAADTAIYYCARFDGEVL | | GNTASLTVSGLQTDDEAVYYCSSLVGNWDVIF |
| | | VYHDWPKPAWVDLWGRGTLVTVSS | | GGGTKLTVL |
| 131 | 393 | QLQMQESGPGLVKPSETLSLSCTVSGDSIRGGEWGDK | 394 | EIVMTQSPDTLSVSPGETVTLSCRASQNINKN |
| | | DYHWGWVRHSAGKGLEWIGSIHWRGTTHYKESLRRRV | | LANYQYKPGQSPRLVIFETYSKIAAFPAREVA |
| | | SMSIDTSRNWFSERLASVTAADTAVYFCARHRHADVE | | SGSGTEFTLTINNMQSEDVAVYYCQQYEEWPR |
| | | MLVPIAGWEDVWGPGVQVTVSS | | TEGQGTKVDIK |
| 132 | 395 | QLQLQESGPGLVKPSETLSLTCTVSGGSMRGTDWGEN | 396 | EIVMTQSPPTLSVSPGETATLSCRASQNVKNN |
| | | DFHYGWIRQSSAKGLEWIGSIHWRGRTTHYKTSERSR | | LAWYQLKPGQAPRLLIFDASSRAGGIPDRFSG |
| | | ATLSIDTSNNRESLTFSFVTAADTAVYYCARHKYHDI | | SGYGTDFTLTVNSVQSEDEGDYFCQQYEEWPR |
| | | FRVVPVAGNFDPWGQGLLVTVSS | | TFGQGTKVDIK |
| 133 | 397 | EVHLEESGPGLVEPSETLSLTCTASGGSIRGGEWGDS | 398 | EIMMTQSPAILSVSPGDRATESCRASQSVKNN |
| | | DYHWGWVRHSPEKGLEWIGSIHWRGTTHYNAPFRGRG | | LAWYQKRPGQAPRLLIFDTSSRASGIPARFSG |
| | | RLSIDLSRNQFSERLTSVTAEDTAVYYCVKHKYADIV | | GGSGTEFTLTVNSMQSEDFATYYCQQYEEWPR |
| | | MVVPIAGWFDPWGQGLQVTVSS | | TFGQGTKVEIK |
| 134 | 399 | QPQLQESGPGLVEASETLSLTCTVSGDSTAACDYFWG | 400 | QSALTQPPSASGSPGQSITISCTGNINNEVSW |
| | | WVRQPPGKGLEWIGSLSHCAGYYNSGWTYHNPSLKSR | | YQQHPGKAPKLVIYGVNKRPSGVPDRFSGSKS |
| | | LTISLDTPKNQVFLKLNSVTAADTAIYYCARFGGDVL | | GNAASLTVSGLQTDDEAVYYCGSLAGNWDVVF |
| | | VYHDWPKPAWVDLWGRGVLVTVSS | | GGGTKLTVL |
| 135 | 401 | QPQLQESGPTLVEASETLSLTCAVSGDSTAACNSFWG | 402 | QSALTQPPSASGSPGQSITISCTGTSNNEVSW |
| | | WVRQPPGKGLEWVGSLSHCASYWNRGWTYHNPSLKSR | | YQQHAGKAPKLVIYDVNKRPSGVPDRFSGSKS |
| | | ITLALDTPKNEVELKLNSVTAADTATYYCARFGGEVL | | GNTASLTVSGLQTDDEAVYYCGSLVGNWDVIF |
| | | RYTDWPKPAWVDLWGRGTLVTVSS | | GGGTKLTVL |
| 136 | 403 | QPQLQESGPGLVEASETLSLTCTVSGDSTAGCDYFWG | 404 | QSALTQPPSASGSPGQSITISCTGTSNNFVSW |
| | | WVRQPPGKGLEWIGGLSHCAGYYNTGWTYHNPSLKSR | | YQQHPAKAPKLVIYGVNKRPSGVPDRFSGSKS |
| | | ITISLDTPKNQVFLKLNSVTAADTAIVYCARFDGEVL | | GNTASLIVSGLQTDDEAVYYCGSLVENWDVIF |
| | | VYNDWPKPAWVDLWGRGTLVTVSS | | GGGTKLTVL |
| 137 | 405 | QVQLQESGPGLVKPAETLSLTCSVSGESINTGHYYWG | 406 | QSALTQPPSASGSLGQSVTISCNGTSSDIGGW |
| | | WVRQVPGKGLEWIGHIHYTTAVLHNPSLKSRLTIKIY | | NEVSWYQQFPGRAPRLIIFEVNKRPSGVPGRF |
| | | TLRNQITLRLSNVTAADTAVYHCVRSGGDILYYYEWQ | | SGSKSGNSASZTVSGLQSDDEGQYECSSLFGR |
| | | KPHWESPWGPGIHVTVSS | | WDVVEGGGTKLTVL |
| 138 | 407 | QVQLRESGPGLVKPSETLSLSCTVSQDSRPSDHSWTW | 408 | WASSELTQPPSVSVSPGQTARITCSGAPLTSR |
| | | VRQSPGKALEWIGDIHYNGATTYNPSLRSRVRIELDQ | | FTYWYRQKPGQAPVLIISRSSQRSSGWSGRFS |
| | | SIPRESLKMTSTAADTGMYYCARNAIRIYGVVALGE | | ASWEGTTVTLTIRGVQADDEADYYCQSSDTSD |
| | | WFHYGNDVWGQGTAVTVSS | | SYKMEGGGTKLTVL |
| 139 | 409 | QVQLRESGPGLVKPSETLSLSCTVSNDSRPSDHSWTW | 410 | SSELTQPPSVSVSPGQTARITCSGAPLTSRFT |
| | | VRQSPGKALEWIGDIHYNGATTYNPSLRSRVRIELDQ | | YWYRQKPGQAPVLIISRSSQRSSGWSGRFSAS |
| | | SIPRFSLKMTSMTAADTGMYYCARNAIRIYGVVALGE | | WSGTTVTLTIRGVQADDEADYYCQSSDTSDSY |
| | | WFHYGMDVWGQGTAVTVSS | | KMEGGGTKLTVL |
| 140 | 411 | EVQLRESGPRLVKPSETLSLSCDVEGDSRPSDHSWTW | 412 | SSELTQAPSVSVSPGQTATIACSEPPLASRYT |
| | | VRQPPGKALEWIGDVHYNGDNTYNPSLRGRVKIDVDR | | YWYRQKPGQAPVLIIFRDRQEPSGVSGRFSAS |
| | | STHRFSLTIKSLTAADTGIYFCARNVIRVEGVISLGE | | KSGTTATLTIRDVQVEDEGDYYCQSSDTSDSY |
| | | WFHYGNDVWGPGTAVIVSS | | KMFGGGTTLTVL |
| 141 | 413 | EVQLRESGPGLVKPSETLSLSCDVEGDSRPSDHSWTW | 414 | SSELTQAPSVSVSPGQTATIACSGPPLASRYT |
| | | VRQPPCKALEWIGDVHYNGDTTYNPSLRGRVKIDVDR | | YWYRQKPGQAPVLIIFRDRQEPSGVSGRFSAS |
| | | STHRFSLTINSLTAADIGIYFCARNVIRVFGVISLGE | | KSGTTATLTIRDVQVEDEGDYYCQSSDTSDSY |
| | | WHYGMDVWGQGTAVTVSS | | KMFGGGTTLTVL |
| 142 | 415 | QVQLRESEPGLVKPSETLSLTCTVSNDSRPSDASWTW | 416 | SSELTQPPSVSVSPGQTAKITCSGAALTSRET |
| | | VRQSPGKALEWIGDIHYNGATTYNPSLRSRVRIELDQ | | YWYRQKPGQAPVLIISRTSQRSSGWSGRFSAS |
| | | SIPRFSLKMTSMTAADIGMYYCARNAIRIYGVVALGE | | WSGTTVTLTIRGVQADDEGDYYCQSSDTSDSY |
| | | WFHYGMDVWGQGTAVTVSS | | KMEGGGTKLTVL |
| 143 | 417 | EVQLRESGPGLVKPSGNMALTCTISGDSRPSDHSWTW | 418 | SSELTQTPSVTVSPGETARIACSGPPLASRYC |
| | | VRQSPGKALEWIGDIHYGGDITYNPSLRSRVKLEVDT | | YWYRQKPGQAPVLIIFRDRQESSCMSGRFASS |
| | | STNRFFLKMTSLTVADTGIYFCARNVIRVEGVIALGE | | HSGTTVTLTIRDVRVEDEADYYCQSSDINDSY |
| | | WFHYGMDVWGQGTAITVSP | | KMFGGGTKVTVL |
| 144 | 419 | EVQLRESGPGLVKPSGNMALTCTISGDSRPSDHSWTW | 420 | SSELTQTASVTVSPGETARIACSGPPLASRYC |
| | | VRQSPGKTLEWIGDIHYGGDITYNPSERSRVKLEVDT | | YWYRQKPGQAPVLIIFRDRQESSGISGRFSSS |
| | | SSNRFFLKMTSLIVADIGIYFCARNVIRVFGVIALGE | | QSGTTVTLTIRDVRVEDEADYYCQSSDTSDSE |
| | | WFHYGMDVWGQGTAITVSP | | KMEGGGTKLTVL |
| 145 | 421 | QVQLRESGPGLVKPSENMALTCTISGDSRPSDHSWTW | 422 | SSELTQAPSVTVSPGDTARIACSGPPLATRYC |
| | | VRQSPGKALEWIGDIHYGGDITYNPSLRSRVELEVDR | | YWYRQKSGQAPVLIIFRDRQESSGVSGRFSSS |
| | | STNRFFLKMTSLSVADTGMYFCARNVIRVEGVIALGE | | QSGSTVTLTIRDVRVEDEADYYCQSSDTSDSY |
| | | WFHYGMDVWGQGTAITVSP | | KMFGGGTKLTVL |
| 146 | 423 | QVQLRESGPGLVKPSETLSLSCDVEGDSRPSDHSWTW | 424 | SSELTQAPSVSVSPGQTARIACSGPPLASRYT |
| | | VRQPPGKALEWIGDIHYNGDKTYNPSLRGRVKIDVDR | | YWYRQKPGQAPVLIIFRDRQFPSGVSGRFSAS |
| | | STHRFSLTENSLTAADTGMYFCARNVIRVEGVISLGE | | KSGTIGTLTIRDVQAEDEGDYYCQSSDTSDSY |
| | | WFHYGMDVWGPGTAVTV | | KMEGGGTTLTVL |
| 147 | 425 | QVQLRESGPGLVKPSGNMALTCTISGDSRPSDHSWTW | 426 | SSELTQAPSVTLSPGETARIACSGPPLASRYC |
| | | VRQSPGKALEWIGDIHYGGDITYNPSLRSRVKLEVDT | | YWYRQKPGQAPVLIIFRDRQESSGISGRFSSS |
| | | SSNRFFLKMISLIVADTGIYECARNVIRVEGVIALGE | | QSGTTVTLTIRDVRVEDEADYYCQSSDNSDSF |
| | | WFHYGMDVWGQGTAITVSP | | KMFGGGTKLTVL |
| 148 | 427 | AEQLVESGGGLVPPGRSLRLSCSASGFYEPDYAMAWV | 428 | DIHMTQSPVSLSASVGDRVTITCRASHFIANY |
| | | RQAPGQGLQWVGFMRGWAYGGSAQFAAFAVGKFAISR | | VNWYQQKPGKAPTLLIFESSTLQRGVPSRFSA |
| | | DDGRNVVYLDVKNPTFEDTGVYFCAREQRNKDYRYGQ | | YGDGTEFTLSINTLQPEDEASYICQQSHSPPV |
| | | EGFGYSYGMDVWGRGTTVVVST | | TFGAGTRVDQK |
| 149 | 429 | EERLVESGGGZVPPGRSLRLSCSAFDFYFPDYAMAWV | 430 | DILMTQSPVSLSASIGERITITCRASHFIANY |
| | | RQAPGKGLEWIGFIRGWAYGQAAQYGKSASGRMTISR | | VNWYQQRPGKAPKLLIFQSWTLNRGIPSRFSG |
| | | DDSRRVVYLDIKSPIEEDTGAYFCAREQRGGDGRYSG | | YGDGTEFTLSISALQSEDEGTYICQQSHSPPL |
| | | DGEGYPYGMDVWGRGTMVTVSA | | SEGGGTRVDQT |
| 150 | 431 | EERLVESGGGLVPPGRSLRLSCSAFDFYEPDYAMAWV | 432 | DIQMTQSPFTLSASVGERVTITCRASHFIANY |
| | | RQAPGRALEWIGFIRGWAYGQSAQYGKSASGRMTISR | | VNWYQQRPGRAPKLLIFESSTLNRGVPSRFSG |
| | | DDSRRVVYLDIKSPTHEDTGVYFCAREQRGANGRYGG | | SGDGTEFTLSISALQSEDEATYICQQSHSPPV |
| | | DGFGYSYGMDVWGRGTMVSVSA | | SFGGGTRVDQT |

In some embodiments, the anti-HIV gp120 V3 glycan-binding antibody comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 7, 8, 9, 10, 11 and 12; SEQ ID NOs.: 7, 13, 9, 10, 11 and 12; SEQ ID NOs.: 14, 15, 16, 17, 11 and 18; SEQ ID NOs.: 14, 19, 20, 17, 11 and 18; SEQ ID NOs.: 21, 22, 23, 24, 25 and 26; SEQ ID NOs.: 21, 22, 27, 24, 25 and 26; SEQ ID NOs.: 28, 29, 30, 31, 32 and 33; SEQ ID NOs.: 34, 35, 36, 37, 25 and 38; SEQ ID NOs.: 39, 40, 41, 42, 43 and 44; SEQ ID NOs.: 45, 46, 47, 48, 49 and 50; SEQ ID NOs.: 45, 51, 52, 53, 49 and 54; SEQ ID NOs.: 55, 56, 57, 58, 59 and 44; SEQ ID NOs.: 60, 46, 61, 58, 49 and 44; SEQ ID NOs: 62, 63, 64, 65, 66 and 67; SEQ ID NOs: 68, 69, 70, 71, 72 and 73; SEQ ID NOs: 74, 75, 76, 77, 78 and 73; SEQ ID NOs: 79, 80, 81, 82, 83 and 73; SEQ ID NOs: 84, 85, 86, 87, 88 and 89; SEQ ID NOs: 84, 90, 91, 92, 93 and 89; SEQ ID NOs: 84, 85, 86, 95, 96 and 89; SEQ ID NOs: 84, 97, 98, 99, 100 and 101; SEQ ID NOs: 84, 97, 98, 99, 100 and 102; SEQ ID NOs: 84, 97, 98, 103, 100 and 89; SEQ ID NOs: 84, 104, 91, 92, 93 and 89; SEQ ID NOs: 84, 97, 98, 99, 100 and 105; SEQ ID NOs: 106, 107, 108, 109, 110 and 111; SEQ ID NOs: 106, 112, 113, 109, 114 and 115 or SEQ ID NOs: 106, 116, 117, 109, 118 and 119 (CDRs according to Kabat).

In some embodiments, the anti-HIV gp120 V3 glycan-binding antibody comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 120, 121, 9, 10, 11 and 12; SEQ ID NOs: 122, 123, 16, 17, 11 and 18; SEQ ID NOs: 124, 125, 20, 17, 11 and 18; SEQ ID NOs: 126, 127, 23, 24, 25 and 26; SEQ ID NOs: 126, 127, 27, 24, 25 and 26; SEQ ID NOs: 128, 192, 30, 31, 32 and 33; SEQ ID NOs: 130, 131, 36, 37, 25 and 38; SEQ ID NOs: 132, 133, 41, 42, 43 and 44: SEQ ID NOs: 134, 135, 47, 48, 49 and 50; SEQ ID NOs: 134, 136, 52, 53, 49 and 54; SEQ ID NOs: 137, 56, 57, 58, 59 and 44; SEQ ID NOs: 138, 135, 61, 58, 49 and 44; SEQ ID NOs: 139, 140, 64, 65, 66 and 67; SEQ ID NOs: 141, 142, 70, 71, 72 and 71; SEQ ID NOs: 143, 144, 76, 77, 78 and 73; SEQ ID NOs: 145, 144, 81, 82, 83 and 73; SEQ ID NOs: 146, 147, 86, 87, 88 and 89; SEQ ID NOs: 148, 147, 86, 87, 88 and 89; SEQ ID NOs: 149, 150, 91, 92, 93 and 89; SEQ ID NOs: 148, 147, 86, 95, 96 and 89; SEQ ID NOs: 149, 151, 98, 99, 100 and 101; SEQ ID NOs: 149, 151, 98, 99, 100 and 102; SEQ ID NOs: 149, 151, 98, 103, 100 and 89; SEQ ID NOs: 149, 151, 98, 99, 100 and 105; SEQ ID NOs: 152, 153, 108, 109, 110 and 111; SEQ ID NOs: 154, 155, 113, 109, 114 and 115; or SEQ ID NOs: 154, 156, 117, 109, 118 and 119 (CDRs according to Chothia).

In some embodiments, the anti-HIV gp120 V3 glycan-binding antibody comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 157, 158, 159, 160, 161 and 12; SEQ ID NOs: 162, 163, 164, 165, 161 and 18; SEQ ID NOs: 162, 163, 166, 165, 161 and 18; SEQ ID NOs: 167, 168, 169, 165, 161 and 18; SEQ ID NOs: 170, 171, 172, 173, 161 and 26; SEQ ID NOs: 170, 171, 174, 173, 161 and 26; SEQ ID NOs: 175, 176, 177, 178, 161 and 38; SEQ ID NOs: 179, 180, 181, 182, 183 and 33; SEQ ID NOs: 184, 185, 186, 187, 188 and 44; SEQ ID NOs: 189, 190, 191, 192, 193 and 50; SEQ ID NOs: 189, 194, 195, 196, 193 and 54; SEQ ID NOs: 197, 198, 199, 200, 201 and 44; SEQ ID NOs: 202, 203, 204, 200, 193 and 44; SEQ ID NOs: 205, 206, 207, 208, 209 and 67; SEQ ID NOs: 210, 211, 212, 213, 214 and 73; SEQ ID NOs: 215, 216, 217, 218, 219 and 73; SEQ ID NOs: 220, 216, 221, 222, 223 and 73; SEQ ID NOs: 224, 225, 86, 226, 227 and 89; SEQ ID NOs: 228, 225, 86, 226, 227 and 89; SEQ ID NOs: 229, 230, 91, 231, 232 and 89; SEQ ID NOs: 229; 233, 91, 231, 232 and 89; SEQ ID NOs: 228, 225, 86, 234, 235 and 89; SEQ ID NOs: 229, 236, 98, 231, 232 and 101; SEQ ID NOs: 229, 236, 98, 231, 232 and 102; SEQ ID NOs: 229, 236, 98, 237, 232 and 89; SEQ ID NOs: 229, 238, 91, 231, 232 and 89; SEQ ID NOs: 229, 236, 98, 231, 232 and 105; SEQ ID NOs: 239, 240, 108, 241, 242 and 111; SEQ ID NOs: 243, 244, 113, 241, 245 and 115; or SEQ ID NOs: 243, 246, 117, 241, 242 and 119 (CDRs according to IMGT).

In some embodiments, the anti-HIV gp120 V3 glycan-binding antibody comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 247, 248, 249, 250, 251 and 252; SEQ ID NOs: 247, 253, 249, 250, 251 and 252; SEQ ID NOs: 254, 255, 256, 257, 251 and 258; SEQ ID NOs: 259, 260, 261, 257, 251 and 258; SEQ ID NOs: 262, 263, 264, 265, 266 and 267; SEQ ID NOs: 262, 263, 268, 265, 266 and 267; SEQ ID NOs: 269, 270, 271, 272, 273 and 274; SEQ ID NOs: 275, 276, 277, 278, 279 and 280; SEQ ID NOs: 281, 282, 283, 284, 285 and 286; SEQ ID NOs: 287, 288, 289, 290, 291 and 286; SEQ ID NOs: 287, 292, 293, 294, 291 and 295; SEQ ID NOs: 296, 297, 298, 299, 300 and 286; SEQ ID NOs: 301, 288, 302, 299, 291 and 286; SEQ ID NOs: 303, 304, 305, 306, 307 and 308; SEQ ID NOs: 309, 310, 311, 312, 313 and 314; SEQ ID NOs: 315, 316, 317, 318, 319 and 314; SEQ ID NOs: 320, 321, 322, 323, 324 and 314; SEQ ID NOs: 325, 326, 327, 328, 329 and 330; SEQ ID NOs: 331, 326, 327, 328, 329 and 330; SEQ ID NOs: 332, 333, 334, 335, 336 and 330; SEQ ID NOs: 332, 337, 334, 335, 336 and 330; SEQ ID NOs: 331, 326, 327, 338, 339 and 330; SEQ ID NOs: 340, 341, 342, 335, 343 and 344; SEQ ID NOs: 340, 341, 342, 335, 345, 346; SEQ ID NOs: 340, 341, 342, 347, 348 and 330; SEQ ID NOs: 332, 349, 334, 335, 336 and 330; SEQ ID NOs: 340, 341, 342, 335, 345 and 350; SEQ ID NOs: 351, 352, 353, 354, 355 and 356 and SEQ ID NOs: 357, 358, 359, 354, 360 and 361; or SEQ ID NOs: 357, 362, 363, 354, 364 and 356 (CDRs according to Honegger).

Illustrative embodiments of CDR sequences of an anti-HIV gp120 V3 glycan-binding antibody, useful in the methods described herein, are provided in Tables A1-A4.

In some embodiments, the anti-HIV gp120 V3 glycan-binding antibody comprises VH and VL comprising amino acid sequences that are at least 80%, at least 85%, at least 90%, at least 91%, at least 92%, at least 93%, at least 94%, at least 95%, at least 96%, at least 97%, at least 98%, at least 99%, or 100%, identical to the amino acid sequences set forth, respectively, as selected from: SEQ ID NOs.: 365 and 366; SEQ ID NOs.: 367 and 368; SEQ ID NOs.: 369 and 370; SEQ ID NOs.: 371 and 372; SEQ ID NOs.: 373 and 374; SEQ ID NOs.: 375 and 376; SEQ ID NOs.: 377 and 378; SEQ ID NOs.: 379 and 380; SEQ ID NOs.: 381 and 382; SEQ ID NOs.: 383 and 384; SEQ ID NOs.: 385 and 386; SEQ ID NOs.: 387 and 388; SEQ ID NOs.: 389 and 390; SEQ ID NOs.: 391 and 392; SEQ ID NOs.: 393 and 394; SEQ ID NOs.: 395 and 396; SEQ ID NOs.: 397 and 398; SEQ ID NOs.: 399 and 400; SEQ ID NOs.: 401 and 402; SEQ ID NOs.: 403 and 404; SEQ ID NOs.: 405 and 406; SEQ ID NOs.: 407 and 408; SEQ ID NOs.: 409 and 410; SEQ ID NOs.: 411 and 412; SEQ ID NOs.: 413 and 414; SEQ ID NOs.: 415 and 416; SEQ ID NOs.: 417 and 418; SEQ ID NOs.: 419 and 420; SEQ ID NOs.: 421 and 422; SEQ ID NOs.: 423 and 424; SEQ ID NOs.: 425 and 426; SEQ ID NOs.: 427 and 428; SEQ ID NOs.: 429 and 430; or SEQ ID NOs.: 431 and 432. Illustrative embodiments of variable domain VH and VL sequences of an anti-HIV gp120 V3 glycan-binding antibody, useful in the methods described herein, are provided in Table B.

In some embodiments, the anti-HIV gp120 V3 glycan-binding antibody is 10-1074-LS. The heavy and light chain amino acid sequences of 10-1074-LS are provided below as SEQ ID NOs: 433 and 434:

Heavy chain:
(SEQ ID NO: 433)
QVQLQESGPGLVKPSETLSVTCSVSGDSMNNYYWTWIRQSPGKGLEWIGYISDRESATYNPSLN
SRVVISRDISKNQLSLKLNSVTPADTAVYYCATARRGQRIYGVVSFGEFFYYYSMDVWGKGTTV
TVSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKENWYVDGVEVHNAKTKPREEQYNSTYRVVSV
LTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVLHEALH
SHYTQKSLSLSPG
Light chain:
(SEQ ID NO: 434)
SYVRPLSVALGETARISCGRQALGSRAVQWYQHRPGQAPILLIYNNQDRPSGIPERFSGTPDIN
FGTRAILTISGVEAGDEADYYCHMWDSRSGFSWSFGGATRLTVLGQPKAAPSVTLFPPSSEELQ
ANKATLVCLISDFYPGAVTVAWKADSSPVKAGVETTTPSKQSNNKYAASSYLSLTPEQWKSHRS
YSCQVTHEGSTVEKTVAPTECS

c. Antibodies Directed to the CD4bs Region of HIV gp120

The CD4 binding site (CD4bs) involves structurally conserved sites located within the β1-α1, loop D, β20-β21 (bridging sheet) and β24-α5 of gp120, which determine the CD4 binding and are involved in the epitopes of CD4bs-binding antibodies (Qiao, et al., Antiviral Res. 2016 August; 132:252-61). The CD4bs of gp120 forms conformational epitopes recognized by anti-CD4bs antibodies involving one or more amino acid residues selected from Thr278, Asp279, Ala281, Thr283, Asp368, Trp427, Glu460, Ser461, Glu462, Leu452, Leu453 and Arg476. The amino acid residues and position numbering is with reference to HXB2 subtype B HIV-1 isolate, which corresponds to residues 1-511 of NCBI Ref Seq No. NP_057856.1, provided below. Residues Thr278, Asp279, Asn280, Ala281, Thr283, Asp368, Trp427, Leu452, Leu453, Gly459, Glu464, Ser465, Glu466, Ile467, Gly472, Gly473 and Arg476, which can contribute to the gp120 CD4bs, are boldened and underlined:

(SEQ ID NO: 435)
MRVKEKYQHLWRWGWRWGTMLLGMLMICSATEKLWVTVYYGVPVWKEATTTLFCASDAKAYDTE
VHNVWATHACVPTDPNPQEVVLVNVTENFNMWKNDMVEQMHEDIISLWDQSLKPCVKLIPLCVS
LKCTDLKNDTNTNSSSGRMIMEKGEIKNCSFNISTSIRGKVQKEYAFFYKLDIIPIDNDTTSYK
LTSCNTSVITQACPKVSFEPIPIHYCAPAGFAILKCNNKTENGTGPCINVSTVQCTHGIRPVVS
TQLLLNGSLAEEEVVIRSVNFTDNAKTIIVQLNISVEINCTRPNNNTRKRIRIQRGPGRAFVTI
GKIGNMRQAHCNISRAKWNNILKQIASKLREQFGNNKTIIFKQSSGGDPEIVTHSFNCGGEFFY
CNSTQLFNSTWENSTWSTEGSNNTEGSDTITLPCRIKQIINMWQKVGKAMYAPPISGQIRCSSN
ITGLLLIRDGGNSNNESEIFRPGGGDMRDNWRSELYKYKVVKIELGVAPTKAKRRVVQREKR.

Tridimensional models depicting amino acid residues contributing to the gp120 CD4bs are provided, e.g., in Canducci, et al., Retrovirology. 2009 Jan. 15; 6:4; Falkowska, et al., J Virol. 2012 April; 86(8):4394-403; and Li, et al., J. Virol. 2012 October; 86(20):11231-41; Gristick, et al., Nat Struct Mol Biol. 2016 October; 23(10):906-915; Kwon, et al., Nat Struct Mol Biol. 2015 July; 22(7):522-31; Liu, et al., Nat Struct Mol Biol. 2017 April; 24(4):370-378; Chen, et al., Science. 2009 Nov. 20; 326(5956):1123-7 and Lyumkis, et al., Science. 2013 Dec. 20; 342(6165):1484-90. In some embodiments, the antibody variants described herein compete with anti-CD4bs antibodies GS-9723, GS-5423, b12, CH103, 1NC9, 12A12, VRC01, VRC07-523, N6, 3BNC117, NIH45-46 and/or PGV04 (VRC-PG04) for binding to gp120 CD4bs. In some embodiments, the antibody variants described herein bind to an overlapping or identical epitope to the epitope bound by anti-CD4bs antibodies GS-9723, GS-5423 (teropavimab), b12, CH103, 1NC9, 12A12, VRC01, VRC07-523, N6, 3BNC117, NIH45-46 and/or PGV04 (VRC-PG04).

Gp120 is encoded by the HIV env gene. The env gene encodes a gene product of around 850 amino acids. The primary env product is the protein gp160, which gets cleaved to gp120 (about 480 amino acids) and gp41 (about 345 amino acids) in the endoplasmic reticulum by the cellular protease furin.

The amino acid sequence of an exemplary gp160 polypeptide of HIV clone identified in NCBI Ref Seq No. NP_057856.1 is provided below (the CD4bs is boldened and underlined):

(SEQ ID NO: 436)
MRVKEKYQHLWRWGWRWGTMLLGMLMICSATEKLWVTVYYGVPVWKEATTTLFCASDAKAYDTE
VHNVWATHACVPTDPNPQEVVLVNVTENFNMWKNDMVEQMHEDIISLWDQSLKPCVKLIPLCVS
LKCTDLKNDTNTNSSSGRMIMEKGEIKNCSFNISTSIRGKVQKEYAFFYKLDIIPIDNDTTSYK
LISCNTSVITQACPKVSFEPIPIHYCAPAGFAILKCNNKTFNGTGPCTNVSTVQCTHGIRPVVS
TQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWENSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKRA
VGIGALFLGFLGAAGSTMGAASMTLTVQARQLLSGIVQQQNNLLRAIEAQQHLLQLTVWGIKQL
QARILAVERYLKDQQLLGIWGCSGKLICITAVPWNASWSNKSLEQIWNHITWMEWDREINNYTS
LIHSLIEESQNQQEKNEQELLELDKWASLWNWFNITNWLWYIKLFIMIVGGLVGLRIVFAVLSI
VNRVRQGYSPLSFQTHLPTPRGPDRPEGIEEEGGERDRDRSIRLVNGSLALIWDDLRSLCLFSY
HRLRDLLLIVTRIVELLGRRGWEALKYWWNLLQYWSQELKNSAVSLLNATAIAVAEGTDRVIEV
VQGACRAIRHIPRRIRQGLERILL

The amino acid sequence of an exemplary gp120 polypeptide of HXB2 subtype B HIV-1 isolate (GenBank Accession No. K0345; corresponding to residues 1-511 of NCBI Ref Seq No. NP_057856.1) is provided below (the CD4bs is boldened and underlined):

(SEQ ID NO: 437)
MRVKEKYQHLWRWGWRWGTMLLGMLMICSATEKLWVTVYYGVPVWKEATTTLFCASDAKAYDTE
VHNVWATHACVPTDPNPQEVVLVNVTENFNMWKNDMVEQMHEDIISLWDQSLKPCVKLTPLCVS
LKCTDLKNDTNTNSSSGRMIMEKGEIKNCSFNISTSIRGKVQKEYAFFYKLDIIPIDNDTTSYK
LTSCNTSVITQACPKVSFEPIPIHYCAPAGFAILKCNNKIFNGIGPCINVSTVQCTHGIRPVVS
TQLLLNGSLAEEEVVIRSVNFTDNAKTIIVQLNTSVEINCTRPNNNTRKRIRIQRGPGRAFVTI
GKIGNMRQAHCNISRAKWNNILKQIASKLREQFGNNKTIIFKQSSGGDPEIVTHSENCGGEFFY
CNSTQLFNSTWENSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR

The amino acid sequence of an exemplary gp120 polypeptide is provided below:

(SEQ ID NO: 438)
AEQLWVTVYYGVPVWREANTTLFCASDAKAYDTEVHNVWATHACVPTDPNPQEVVMGNVTEDEN
MWKNNMVEQMHEDIISLWDQSLKPCVKLTPLCVTLHCTNVTISSINGSTANVTMREEMKNCSEN
TTTVIRDKIQKEYALFYKLDIVPIEGKNINISYRLINCNTSVITQACPKVSFEPIPIHYCAPAG
FAILKCNNKTFNGKGPCRNVSTVQCTHGIKPVVSTQLLINGSLAEEDIIIRSENFTNNGKNIIV
QLKEPVKINCTRPGNNTRRSINIGPGRAFYATGAIIGDIRKAHCNISTEQWNNTLTQIVDKLRE
QFGNKTIIFNQSSGGDPEVVMHTFNCGGEFFYCNSTQLENSTWENNGISTWNSTADNITLPCRI
KQVINMWQEVGKAMYAPPIRGQIDCSSNITGLILIRDGGSNSSQNETFRPGGGNMKDNWRSELY
KYKVVKIEPLGIAPTRAKRRVVQREKR.

The amino acid sequence of another exemplary gp120 polypeptide (see, bioafrica.net/proteomics/ENV-GP120prot.html) is provided below:

(SEQ ID NO: 439)
TEKLWVTVYYGVPVWKEATTTLFCASDAKAYDTEVHNVWATHACVPTDPNPQEVVLVNVTENEN
MWKNDMVEQMHEDIISLWDQSLKPCVKLTPLCVSLKCTDLKNDININSSSGRMIMEKGEIKNCS
FNISTSIRGKVQKEYAFFYKLDIIPIDNDTTSYKLISCNTSVITQACPKVSFEPIPIHYCAPAG
FAILKCNNKTFNGTGPCTNVSTVQCTHGIRPVVSTQLLINGSLAEEEVVIRSVNFTDNAKTIIV
QLNISVEINCTRPNNNTRKRIRIQRGPGRAFVTIGKIGNMRQAHCNISRAKWNNILKQIASKLR
EQFGNNKTIIFKQSSGGDPEIVTHSFNCGGEFFYCNSTQLENSTWENSTWSTEGSNNTEGSDTI
TLPCRIKQIINMWQKVGKAMYAPPISGQIRCSSNITGLLLTRDGGNSNNESEIFRPGGGDMRDN
WRSELYKYKVVKIEPLGVAPTKAKRRVVQREKR

In certain embodiments of the methods described herein, the subject is administered an antibody that binds to HIV gp120 protein within the CD4bs region, e.g., an epitope or region of gp120 CD4 binding site. In certain embodiments, the administered antibody binds to HIV-1 antigens expressed on a cell surface and eliminates or kills the infected cell.

Illustrative broadly neutralizing antibodies that bind to gp120 in the CD4bs and which can be used in the herein described methods include without limitation from an antibody selected from the group consisting of 3BNC117, GS-9723, GS-5423, 3BNC60, b12, F105, VRC01, VRC07, VRC07-523, VRC03, VRC06, VRC06b01 VRC08, VRC0801, NIH45-46, PGV04 (VRC-PG04); CH103, 44-VRC13.01, 1NC9, 12A12, N6, 1-18, N49-P7, NC-Cow1, IOMA, CH235 and CH235.12, N49P6, N49P7, N49P11, N49P9 and N60P25.

Illustrative sequences of complementarity determining regions (CDRs) of the antibody targeting HIV gp120 CD4bs region, useful in the methods described herein, are provided in Tables C1-C4. Illustrative sequences of the VH and VL of the antibody targeting HIV gp120 CD4bs region, useful in the methods described herein, are provided in Table D.

TABLE C1
CDRs (Kabat) for illustrative anti-HIV gp120 CD4bs antibodies

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 151 | DYFIH | WINPKTGQPNNPRQFQG | QRSDYWDFDV | QANGYLN | DGSKLER | QVYEF |
| | SEQ ID | SEQ ID NO: 443 | SEQ ID NO: 444 | SEQ ID | SEQ ID | SEQ ID |
| | NO: 442 | | | NO: 445 | NC: 446 | NO: 447 |
| 152 | DHFIH | WINPKTGQPNNPRQFQG | QRSDFWDFDV | QANGYLN | DESKLER | QVYEF |
| | SEQ ID | SEQ ID NO: 443 | SEQ ID NO: 449 | SEQ ID | SEQ ID | SEQ ID |
| | NO: 448 | | | NO: 445 | NO: 446 | NO: 447 |
| 153 | NCPIN | WNKPRGGAVSYARQLQG | GKYCTARDYYNWDFEH | RTSQYGSLA | SESTRAA | QQYEF |
| | SEQ ID | SEQ ID NO: 451 | SEQ ID NO: 452 | SEQ ID | SEQ ID | SEQ ID |
| | NO: 450 | | | NO: 453 | NO: 454 | NO: 455 |
| 154 | NCPIN | WKPRHGAVSYARQLQG | GKYCTARDYYNWDFEH | RTSQYGSLA | SGSTRAA | QQYEF |
| | SEQ ID | SEQ ID NO: 456 | SEQ ID NO: 452 | SEQ ID | SEQ ID | SEQ ID |
| | NO: 450 | | | NO: 453 | NO: 454 | NO: 455 |
| 155 | DCTLN | WLKPRGGAVNYARPLQ | GKNCDYNWDFEH | RTSQYGSLA | SESTRAA | QQYEF |
| | SEQ ID | SEQ ID NO: 458 | SEQ ID NO: 459 | SEQ ID | SEQ ID | SEQ ID |
| | NO: 457 | | | NO: 453 | NO: 454 | NO: 455 |
| 156 | AHILF | WIKPQYGAVNFCCCFRD | DRSYGDSSWALDA | QTSQCVCSDLH | HISSVED | QVLQF |
| | SEQ ID | SEQ ID NO: 461 | SEQ ID NO: 462 | SEQ ID | SEQ ID | SEQ ID |
| | NO: 460 | | | NO: 463 | NO: 464 | NO: 465 |
| 157 | DDDTFTKYWTH | VISPHFARPIYSYKFRD | DPFGDRAPHYNYHMDV | RASQGLDSSHLA | GTSNRAR | QRYGGTPIT |
| | SEQ ID | SEQ ID NO: 467 | SEQ ID NO: 468 | SEQ ID | SEQ ID | SEQ ID |
| | NO: 466 | | | NO: 469 | NO: 470 | NO: 471 |
| 158 | RTELIH | WVKTVTGAVNFGSPDFR | QKFYTGGQGWYFDL | TAASYGHMT | ATSKRAS | QQLEF |
| | SEQ ID | SEQ ID NO: 173 | SEQ ID NO: 174 | SEQ ID | SEQ ID | SEQ ID |
| | NO: 472 | | | NO: 475 | NO: 476 | NO: 477 |

TABLE C2
CDRs (Chothia) for illustrative anti-HIV gp120 CD4bs antibodies

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 159 | GYNIRDY | PKTG | RSDYWDFD | NGY | DGS | YE |
| | SEQ ID NO: 478 | SEQ ID NO: 479 | SEQ ID NO: 480 | SEQ ID | SEQ ID | SEQ ID |
| | | | | NO: 481 | NO: 482 | NO: 483 |
| 160 | GYKISDH | PKTG | RSDFWDFD | NGY | DGS | YE |
| | SEQ ID NO: 484 | SEQ ID NO: 479 | SEQ ID NO: 485 | SEQ ID | SEQ ID | SEQ ID |
| | | | | NO: 481 | NO: 482 | NO: 483 |
| 161 | GYEFINC | PRGG | KYCTARDYYNWDFE | SQYGS | SGS | YE |
| | SEQ ID NO: 486 | SEQ ID NO: 487 | SEQ ID NO: 488 | SEQ ID | SEQ ID | SEQ ID |
| | | | | NO: 489 | NO: 490 | NO: 483 |
| 162 | GYEFINC | PRHG | KYCTARDYYNWDFE | SQYGS | SGS | YE |
| | SEQ ID NO: 486 | SEQ ID NO: 491 | SEQ ID NO: 488 | SEQ ID | SEQ ID | SEQ ID |
| | | | | NO: 489 | NO: 490 | NO: 483 |
| 163 | GYEFIDC | PRGG | KNCDYNWDFE | SQYGS | SGS | YE |
| | SEQ ID NO: 492 | SEQ ID NO: 487 | SEQ ID NO: 493 | SEQ ID | SEQ ID | SEQ ID |
| | | | | NO: 489 | NO: 490 | NO: 483 |
| 164 | GYTFTAH | PQYG | RSYGDSSWALD | SQGVGSD | HTS | LQ |
| | SEQ ID NO: 494 | SEQ ID NO: 495 | SEQ ID NO: 496 | SEQ ID | SEQ ID | SEQ ID |
| | | | | NO: 497 | NO: 498 | NO: 499 |
| 165 | DDPYTDDDTFTKY | PHFA | PFGDRAPHYNYHMD | SQGLDSSH | GTS | YGGTPI |
| | SEQ ID NO: 500 | SEQ ID NO: 501 | SEQ ID NO: 502 | SEQ ID | SEQ ID | SEQ ID |
| | | | | NO: 503 | NO: 504 | NO: 505 |
| 166 | EDIFERTE | TVTG | KFYTGGQGWYFD | ASYGH | ATS | LE |
| | SEQ ID NO: 506 | SEQ ID NO: 507 | SEQ ID NO: 508 | SEQ ID | SEQ ID | SEQ ID |
| | | | | NO: 509 | NO: 510 | NO: 511 |

TABLE C3
CDRs (IMGT) for illustrative anti-HIV gp120 CD4bs antibodies

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 167 | CYNIKDYF | INPKTGQP | ARQRSDYWDFDV | NCY | DCS | QVYEF |
| | SEQ ID NO: 512 | SEQ ID NO: 513 | SEQ ID NO: 514 | SEQ ID | SEQ ID | SEQ ID |
| | | | | NO: 481 | NO: 482 | NO: 447 |
| 168 | GYKISDHF | INPKTGQP | ARQRSDFWDFDV | NGY | DGS | QVYEF |
| | SEQ ID NO: 515 | SEQ ID NO: 513 | SEQ ID NO: 516 | SEQ ID | SEQ ID | SEQ ID |
| | | | | NO: 481 | NO: 482 | NO: 447 |
| 169 | GYEFINCP | MKPRGGAV | TRGKYCTARDYYNWDFEH | QYGS | SGS | QQYEF |
| | SEQ ID NO: 517 | SEQ ID NO: 518 | SEQ ID NO: 519 | SEQ ID | SEQ ID | SEQ ID |
| | | | | NO: 520 | NO: 490 | NO: 455 |
| 170 | GYEFINCP | MKPRAGAV | TRGKYCTARDYYNWDFEH | QYGS | SGS | QQYEF |
| | SEQ ID NO: 517 | SEQ ID NO: 521 | SEQ ID NO: 519 | SEQ ID | SEQ ID | SEQ ID |
| | | | | NO: 520 | NO: 490 | NO: 455 |
| 171 | GYEFIDCT | LKPRGGAV | TRGKNCDYNWDFEH | QYGS | SGS | QQYEF |
| | SEQ ID NO: 522 | SEQ ID NO: 523 | SEQ ID NO: 524 | SEQ ID | SEQ ID | SEQ ID |
| | | | | NO: 520 | NO: 490 | NO: 455 |
| 172 | GYTFTAHI | IKPQYCAV | ARDRSYGDSSWALDA | QCVCSD | HTS | QVLQF |
| | SEQ ID NO: 525 | SEQ ID NO: 526 | SEQ ID NO: 527 | SEQ ID | SEQ ID | SEQ ID |
| | | | | NO: 528 | NO: 498 | NO: 465 |
| 173 | DDPYTDDDIFTKYW | ISPHFARP | ARDFFGDRAPHYNYHMDV | QGLDSSH | GTS | QRYGGTPIT |
| | SEQ ID NO: 529 | SEQ ID NO: 530 | SEQ ID NO: 531 | SEQ ID | SEQ ID | SEQ ID |
| | | | | NO: 532 | NO: 504 | NO: 471 |
| 174 | EDIFERTEL | VKTVTGAV | ARQKFYTGGQGWYFDL | SYGH | ATS | QQLEF |
| | SEQ ID NO: 533 | SEQ ID NO: 534 | SEQ ID NO: 535 | SEQ ID | SEQ ID | SEQ ID |
| | | | | NO: 536 | NO: 510 | NO: 477 |

TABLE C4
CDRs (Honegger) for illustrative anti-HIV gp120 CD4bs antibodies

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 175 | ASGYNIRDYF | INPKTGQPNNPRQFQGR | QRSDYWDFD | ANGY | DGSKLERGVPSRF | YE |
| | SEQ ID NO: 538 | SEQ ID NO: 530 | SEQ ID | SEQ ID | SEQ ID NO: 542 | SEQ ID |
| | | | NO: 540 | NO: 541 | | NO: 483 |
| 176 | ASGYKISDHF | INPKTGQPNNPRQFQGR | QRSDFWDFD | ANGY | DGSKLERGVPAR | YE |
| | SEQ ID NO: 543 | SEQ ID NO: 539 | SEQ ID | SEQ ID | SEQ ID NO: 545 | SEQ ID |
| | | | NO: 544 | NO: 541 | | NO: 483 |
| 177 | ASGYEFTNCP | MKPRGGAVSYARQLQGR | GKYCTARDYYNW | TSQYGS | SGSTRAAGIPDR | YE |
| | SEQ ID NO: 546 | SEQ ID NO: 547 | DFE | SEQ ID | SEQ ID NO: 550 | SEQ ID |
| | | | SEQ ID | NO: 549 | | NO: 483 |
| | | | NO: 548 | | | |
| 178 | ASGYEFTNCP | MKPRHGAVSYARQLQGR | GKYCTARDYYNW | TSQYGS | SGSTRAAGIPDR | YE |
| | SEQ ID NO: 546 | SEQ ID NO: 551 | DFE | SEQ ID | SEQ ID NO: 550 | SEQ ID |
| | | | SEQ ID | NO: 549 | | NO: 483 |
| | | | NO: 548 | | | |
| 179 | ASGYEFIDCT | LKPRGGAVNYARPLQGR | GKNCDYNWDFE | TSQYGS | SGSTRAAGIPDR | YE |
| | SEQ ID NO: 552 | SEQ ID NO: 553 | SEQ ID | SEQ ID | SEQ ID NO: 550 | SEQ ID |
| | | | NO: 554 | NO: 549 | | NO: 483 |
| 180 | TSGYTFTAHI | IKPQYGAVNFGGGFRDR | DRSYGDSSWALD | TSQGVGSD | HTSSVEDGVPSR | LQ |
| | SEQ ID NO: 555 | SEQ ID NO: 556 | SEQ ID | SEQ ID | SEQ ID NO: 559 | SEQ ID |
| | | | NO: 557 | NO: 558 | | NO: 499 |
| 181 | ADDDFYTDDDTFT | ISPHFARPIYSYKERDR | DPEGDRAPHYNY | ASQGLDSSH | GTSNRARGTPDR | YGGTEI |
| | KYW | SEQ ID NO: 561 | HMD | SEQ ID | SEQ ID NO: 564 | SEQ ID |
| | SEQ ID NO: 360 | | SEQ ID | NO: 563 | | NO: 505 |
| | | | NO: 562 | | | |
| 182 | TSEDIFERTEL | VKTVTGAVNFGSPDERQ | QKFYTGGOGWYED | AASYGH | ATSKRASGIPDR | LE |
| | SEQ ID NO: 565 | SEQ ID NO: 566 | SEQ ID | SEQ ID | SEQ ID NO: 569 | SEQ ID |
| | | | NO: 567 | NO: 568 | | NO: 511 |

TABLE D
VH/VL for illustrative anti-HIV gp120 CD4bs antibodies

| | SEQ | | SEQ | |
|---|---|---|---|---|
| Ab | ID | | ID | |
| Name | NO | VH | NO | VL |
| 183 | 571 | QVQLLQSGAAVTKPGASVRVSCEASGYNIRDYF | 572 | DIQMTQSPSSLSASVGDTVTITCQANGYLNWYQQR |
| | | IHWWRQAPGQGLQWVGWINPKTGQPNNPRQFQG | | RGKAPKLLIYDGSKLERGVPSRFSGRRWGQEYNLT |
| | | RVSLTRHASWDFDTFSFYMDLKALRSDDTAVYF | | INNLQPEDIATYFCQVYEFVVPGTRLDLK |
| | | CARQRSDYWDFDVWGSGTQVTVSS | | |
| 184 | 573 | QVQLLQSGAAVTKPGASVRVSCEASGYNIRDYF | 574 | DIQWTQSPSSLSASVGDTATITCQANGYLNWYQQR |
| | | IHWWRQAPGQGLQWVGWINPKTGQPNNPRQFQG | | RGKAPKLLIYDGSKLERGVPSRFSGRRWGQEYNLT |
| | | RVSLTRHASFDFDTFSFYMDLKALRSDDTAVYF | | INNLQPEDIATYFCQVYEFVVPGTRLDLK |
| | | CARQRSDYWDFDVWGSGTQVTVSS | | |
| 185 | 575 | QVHLSQSGAAVTKPGASVRVSCEASGYKISDHF | 576 | DIQMTQSPSSLSARVGDTVTITCQANGYLNWYQQR |
| | | IHWWRQAPGQGLQWVGWINPKTGQPNNPRQFQG | | RGKAPKLLIYDGSKLERGVPARFSGRRWGQEYNLT |
| | | RVSLTRQASWDFDTYSFYMDLKAVRSDDTAIYF | | INNLQPEDVATYFCQVYEFIVPGTRLDLK |
| | | CARQRSDFWDFDVWGSGTQVTVSS | | |
| 186 | 577 | QVRLSQSGGQMKKPGDSMRISCRASGYEFINCP | 578 | EIVLTQSPGTLSLSPGETAIISCRTSQYGSLAWYQ |
| | | INWIRLAPGKRPEWMGWMKPRGGAVSYARQLQG | | QRPGQAPRLVIYSGSTRAAGIPDRFSGSRWGPDYN |
| | | RVTMTRDMYSETAFLELRSLTSDDTAVYFCTRG | | LTISNLESGDEGVYYCQQYEFFGQGTKVQVDIK |
| | | KYCTARDYYNWDFEHWGQGTPVTVSS | | |
| 187 | 579 | QVRLSQSGGQMKKPGDSMRISCRASGYEFINCP | 580 | SLTQSPGTLSLSPGETAIISCRTSQYGSLAWYQQR |
| | | INWIRLAPGKRPEWMGWMKPRHGAVSYARQLQG | | PGQAPRAVIYSGSTRAAGIPDRFSGSRWGPDYNLT |
| | | RVTMTRDMYSETAFLELRSLTSDDTAVYFCTRG | | ISNLESGDEGVYYCQQYEFFGQGTKVQVDIK |
| | | KYCTARDYYNWDFEHWGQGTPVTVSS | | |
| 188 | 581 | QVQLVQSGGQMKKPGESMRISCRASGYEFIDCT | 582 | EIVLTQSPGTLSLSPGETAIISCRTSQYGSLAWYQ |
| | | ENWIRLAPGKRPEWMGWLKPRGGAVNYARPLQG | | QRPGQAPRLVIYSGSTRAAGIPDRFSGSRWGPDYN |
| | | RVTMTRDVYSDTAFLELRSLTVDDTAVYFCTRG | | ITISNLESGDFGVYYCQQYEFFGQGTKVQVDIK |
| | | KNCDYNWDFEHWGRGTPVIVSS | | |
| 189 | 583 | RAHLVQSGTAMKKPGASVRVSCQTSGYTFTAHI | 584 | YIHVTQSPSSLSVSIGDRVTINCQTSQGVGSDLHW |
| | | LEWFRQAPGRGLEWVGWIKPQYGAVNFGGGERD | | YQHKPGRAPKLLIHHTSSVEDGVPSRFSGSGEHTS |
| | | RVTLTRDVYREIAYMDIRGLKPDDTAVYYCARD | | ENLTISDLQADDIATYYCQVLQFFGRGSRLHIK |
| | | RSYGDSSWALDAWGQGTTVVVSA | | |
| 190 | 585 | QGRLFQSGAEVKRPGASVRISCRADDDPYTDDD | 586 | EVVLTQSPAILSVSPGDRVILSCRASQGLDSSHLA |
| | | TFTKYWTHWIRQAPGQRPEWIGVISPHFARPIY | | WYRFKRGQIPTLVIFGTSNRARGTPDRFSGSGSGA |
| | | SYKFRDRLTLTRDSSLTAVYLELKGLQPDDSGI | | DETLTISRVEPEDFATYYCQRYGGTPITFGGGTTL |
| | | YFCARDPFGDRAPHYNYHMDVWGGGTAVIVSS | | DKKRTVA |
| 191 | 587 | QVQLVQSGSGVKKPGASVRVSCWTSEDIFERTE | 588 | EIVLTQSPGTLSLSPGETASLSCTAASYGHMTWYQ |
| | | LIHWVRQAPGQGLEWIGWVKTVTGAVNFGSPDF | | KKPGQPPKLLIFATSKRASGIPDRFSGSQFGKQYT |
| | | RQRVSLTRDRDLFTAHMDIRGLTQGDTATYFCA | | LTITRMEPEDFARYYCQQLEFFGQGTRLEIRRTVA |
| | | RQKFYTGGQGWYFDLWGRGTLIVVSS | | |

In some embodiments, the anti-HIV gp120 CD4bs-binding antibody comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 442, 443, 444, 445, 446 and 447; SEQ ID NOs.: 448, 443, 449, 445, 446 and 447; SEQ ID NOs.: 450, 451, 452, 453, 454 and 455; SEQ ID NOs.: 450, 456, 452, 453, 454, 455; SEQ ID NOs.: 457, 458, 459, 453, 454 and 455; SEQ ID NOs.: 460, 461, 462, 463, 464 and 465; SEQ ID NOs.: 466, 467, 468, 469, 470 and 471; or SEQ ID NOs.: 472, 473, 474, 475, 476 and 477 (CDRs according to Kabat).

In some embodiments, the anti-HIV gp120 CD4bs-binding antibody comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 478, 479, 480, 481, 482 and 483; SEQ ID NOs.: 484, 479, 485, 481, 482 and 483; SEQ ID NOs.: 486, 487, 488, 489, 490 and 483; SEQ ID NOs.: 486, 491, 488, 489, 490 and 483; SEQ ID NOs.: 492, 487, 493, 489, 490 and 483; SEQ ID NOs.: 494, 495, 496, 497, 498 and 499; SEQ ID NOs.: 500, 501, 502, 503, 504 and 505; or SEQ ID NOs.: 506, 507, 508, 509, 510 and 511 (CDRs according to Chothia).

In some embodiments, the anti-HIV gp120 CD4bs-binding antibody comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 512, 513, 514, 481, 482 and 447; SEQ ID NOs.: 515, 513, 516, 481, 482 and 447; SEQ ID NOs.: 517, 518, 519, 520, 490 and 455; SEQ ID NOs.: 517, 522, 519, 520, 521 and 455; SEQ ID NOs.:522, 523, 524, 520, 490 and 455; SEQ ID NOs: 525, 526, 527, 528, 498 and 465; SEQ ID NOs: 529, 530, 531, 532, 504 and 471; SEQ ID NOs: 533, 534, 535, 536, 510 and 477 (CDRs according to IMGT).

In some embodiments, the anti-HIV gp120 CD4bs-binding antibody comprises a VH comprising a VH-CDR1, a VH-CDR2, and a VH-CDR3; and a VL comprising a VL-CDR1, a VL-CDR2, and a second VH-CDR3; wherein the VH-CDR1, the VH-CDR2, the VH-CDR3 the VL-CDR1, the VL-CDR2, and the VH-CDR3 comprise the sequences set forth in: SEQ ID NOs.: 538, 539, 540, 541, 542 and 483; SEQ ID NOs.: 543, 539, 544, 541, 545 and 483; SEQ ID NOs.: 546, 547, 548, 549, 550 and 483; SEQ ID NOs.: 546, 551, 548, 549, 550 and 483; SEQ ID NOs.: 555, 556, 557, 558, 559 and 499; SEQ ID NOs: 560, 561, 562, 563, 564 and 505; SEQ ID NOs: 565, 566, 567, 568, 569, 569 and 511 (CDRs according to Honegger).

In some embodiments, the anti-HIV gp120 CD4bs-binding antibody comprises VH and VL comprising amino acid sequences that are at least 80%, at least 85%, at least 90%, at least 91%, at least 92%, at least 93%, at least 94%, at least 95%, at least 96%, at least 97%, at least 98%, at least 99%, or 100%, identical to the amino acid sequences set forth, respectively, as selected from: SEQ ID NOs.: 571 and 572; SEQ ID NOs.: 573 and 574; SEQ ID NOs.: 575 and 576; SEQ ID NOs.: 577 and 578; SEQ ID NOs.: 579 and 580; SEQ ID NOs.:581 and 582; SEQ ID NOs.: 583 and 584; or SEQ ID NOs.:585 and 586; 587 and 588.

In some embodiments, the anti-HIV gp120 CD4bs-binding antibody is 3BNC117-LS. The heavy and light chain amino acid sequences of 3BNC117-LS are provided below as SEQ ID NOs: 589 and 590:

Heavy chain:
(SEQ ID NO: 589)
QVQLLQSGAAVTKPGASVRVSCEASGYNIRDYFIHWWRQAPGQGLQWVGWINPKTGQPNNPRQF
QGRVSLTRHASWDFDTFSFYMDLKALRSDDTAVYFCARQRSDYWDFDVWGSGTQVTVSSASTKG
PSVFPLAPSSKSTSGGTAALGCLVKDYFPEPVTVSWNSGALISGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLIVLHQDWL
NGKEYKCKVSNKALPAPIEKTISKAKGQPREPQVYTLPPSRDELTKNQVSLTCLVKGFYPSDIA
VEWESNGQPENNYKITPPVLDSDGSFFLYSKLTVDKSRWQQGNVFSCSVLHEALHSHYTQKSLS
LSPG
Light chain:
(SEQ ID NO: 590)
DIQMTQSPSSLSASVGDTVTITCQANGYLNWYQQRRGKAPKLLIYDGSKLERGVPSRFSGRRWG
QEYNLTINNLQPEDIATYFCQVYEFVVPGTRLDLKRIVAAPSVFIFPPSDEQLKSGTASVVCLL
NNFYPREAKVQWKVDNALQSGNSQESVTEQDSKDSTYSLSSTLILSKADYEKHKVYACEVTHQG
LSSPVTKSENRGEC

d. Fc Amino Acid Substitutions that Increase Serum Half-Life

In some embodiments, the Fc region or Fc domain of the anti-HIV gp120 bNAbs comprise amino acid modifications that promote an increased serum half-life of the anti-binding molecule. Amino acid substitutions that increase the half-life of an antibody have been described. In one embodiment, the Fc region or Fc domain of one or both of s heavy chains comprise a methionine to tyrosine substitution at position 252 (EU numbering), a serine to threonine substitution at position 254 (EU numbering), and a threonine to glutamic acid substitution at position 256 (EU numbering). See, e.g., U.S. Pat. No. 7,658,921. This type of mutant, designated as a “YTE” exhibits a four-fold increased half-life relative to wild-type versions of the same antibody (Dall'Acqua, et al., J Biol Chem, 281: 23514-24 (2006); Robbie, et al., Antimicrob Agents Chemotherap., 57(12):6147-6153 (2013)). In certain embodiments, the Fc region or Fc domain of one or both heavy chains comprise an IgG constant domain comprising one, two, three or more amino acid substitutions of amino acid residues at positions 251-257, 285-290, 308-314, 385-389, and 428-436 (EU numbering). Alternatively, M428L and N434S (“LS”) amino acid substitutions can increase the pharmacokinetic half-life of the multi-specific antigen binding molecule. In other embodiments, the Fc region or Fc domain of one or both heavy chains comprise a M428L and N434S substitution (EU numbering). In other embodiments, the Fc region or Fc domain of one or both heavy chains comprise T250Q and M428L (EU numbering) amino acid substitutions, e.g., as described in U.S. Pat. Nos. 7,217,797 and 7,217,798. In other embodiments, the Fc region or Fc domain of one or both heavy chains comprise H433K and N434F (EU numbering) amino acid substitutions, e.g., as described in U.S. Pat. No. 8,163,881. In other embodiments, the Fc region or Fc domain of one or both heavy chains comprise T307Q/Q311V/A378V (DF215) or T256D/N286D/T307R/Q311V/A378V (DF228) (EU numbering) amino acid substitutions, e.g., as described in U.S. Patent Publ. No. 2020-0277358. In some embodiments, the Fc region or Fc domain of one or both heavy chains comprise aspartic acid at position 309, histidine at position 311 and serine at position 434 (DHS), e.g., as described in U.S. Pat. No. 11,059,892.

3. Scheduling Regimen

Generally, the present methods entail treating or preventing HIV in a human subject in need thereof by co-administering twice annually an effective amount of bNAb that binds to an epitope of gp120 within the third variable loop (V3) and/or high mannose patch comprising a N332 oligomannose glycan and an effective amount of a bNAb that binds to an epitope of gp120 comprising the CD4 binding site (CD4bs), both bNAbs having Fc amino acid substitutions to extend serum half-life. In various embodiments, the cadence of co-administrations can be once every six months (i.e., Q6M), once every 24 weeks (i.e., Q24W), once every 25 weeks (i.e., Q25W), once every 26 weeks (i.e., Q26W).

A “subject,” “individual” or “patient” refers to any mammal, including humans and non-human primates. In particular embodiments, the mammal is human.

“Effective amount” or “therapeutically effective amount” refers to that amount of an antibody that, when administered alone or in combination with another therapeutic agent to a cell, tissue, or subject is sufficient to effect treatment or a beneficial result in the subject. The amount which constitutes an “effective amount” will vary depending on the antibody and its specific use, and potentially also the condition and its severity, the manner of administration, and the age of the subject to be treated, but can be determined routinely by one of ordinary skill in the art having regard to his own knowledge and to this disclosure. A therapeutically effective dose further refers to that amount of the antibody sufficient to treat, prevent or ameliorate an infection or disease condition or the progression of an infection or disease, and that amount sufficient to effect an increase in rate of treatment, healing, prevention or amelioration of such conditions. When applied to an individual antibody administered alone, a therapeutically effective dose refers to that active ingredient alone. When applied to a combination, a therapeutically effective dose refers to combined amounts of the active ingredients that result in the therapeutic effect, whether administered in combination, serially or simultaneously. In some embodiments, a therapeutically effective dose allows for an efficacious blood or serum concentration of antibody at the time of a second or subsequent administration (e.g., at 6 months, 24 weeks, 25 weeks or 26 weeks after a first or prior administration).

In certain embodiments, the anti-HIV gp120 V3 glycan binding antibody and the anti-HIV gp120 CD4bs binding antibody, described herein, are each administered intravenously in a therapeutically effective dosage amount in the range of from about 500 mg to about 3000 mg, e.g., from about 550 mg to about 2900 mg, e.g., from about 600 mg to about 2800 mg, e.g., from about 650 mg to about 2700 mg, e.g., from about 700 mg to about 2600 mg, e.g., from about 850 mg to about 2550 mg. In some embodiments, the anti-HIV gp120 V3 glycan binding antibody (e.g., 10-1074-LS) is administered intravenously at a dose of 850 mg. In some embodiments, the anti-HIV gp120 V3 glycan binding antibody (e.g., 10-1074-LS) is administered intravenously at a dose of 2550 mg. In some embodiments, the anti-HIV gp120 CD4bs binding antibody (e.g., 3BNC117-LS) is administered intravenously at a dose of 1700 mg. In some embodiments, the anti-HIV gp120 CD4bs binding antibody (e.g., 3BNC117-LS) is administered intravenously at a dose of 2550 mg. In some embodiments, the anti-HIV gp120 V3 glycan binding antibody (e.g., 10-1074-LS) is administered intravenously at a dose of 2550 mg and the anti-HIV gp120 CD4bs binding antibody (e.g., 3BNC117-LS) is administered intravenously at a dose of 2550 mg. In some embodiments, the anti-HIV gp120 V3 glycan binding antibody (e.g., 10-1074-LS) is administered intravenously at a dose of 850 mg and the anti-HIV gp120 CD4bs binding antibody (e.g., 3BNC117-LS) is administered intravenously at a dose of 2550 mg. In some embodiments, the anti-HIV gp120 V3 glycan binding antibody (e.g., 10-1074-LS) is administered intravenously at a dose of 850 mg and the anti-HIV gp120 CD4bs binding antibody (e.g., 3BNC117-LS) is administered at a dose of 1700 mg. In some embodiments, the anti-HIV gp120 V3 glycan binding antibody (e.g., 10-1074-LS) is administered intravenously at a dose of 850 mg and the anti-HIV gp120 CD4bs binding antibody (e.g., 3BNC117-LS) is administered at a dose of 1275 mg. In some embodiments, the anti-HIV gp120 V3 glycan binding antibody (e.g., 10-1074-LS) is administered intravenously at a dose of 10 mg/kg and the anti-HIV gp120 V3 glycan and anti-HIV gp120 CD4bs binding antibodies (e.g., 3BNC117-LS) is administered intravenously at a dose of 30 mg/kg.

“Treat,” “treating” or “treatment” as used herein covers the treatment of the disease, injury, or condition of interest, e.g., HIV-1 infection, in a subject, e.g., a mammal, such as a human, having the disease or condition of interest, and includes: (i) inhibiting progression of the disease, injury, or condition, i.e., arresting its development; (ii) reducing or relieving the disease, injury, or condition, i.e., causing regression of the disease or condition; or (iii) relieving the symptoms resulting from the disease, injury, or condition. As used herein, the terms “disease,” “disorder,” and “condition” may be used interchangeably. As used herein, “inhibition,” “treatment,” “treating,” and “ameliorating” are used interchangeably and refer to, e.g., stasis of symptoms, prolongation of survival, partial or full amelioration of symptoms, and partial or full eradication of a condition, disease or disorder.

As used herein, “prevent” or “prevention” includes (i) preventing or inhibiting the disease, injury, or condition from occurring in a subject, in particular, when such subject is predisposed to the condition but has not yet been diagnosed as having it; or (ii) reducing the likelihood that the disease, injury, or condition will occur in the subject.

Co-administration includes concurrent administration as well as administration of unit dosages of the anti-HIV gp120 V3 glycan binding antibody and the anti-HIV gp120 CD4bs binding antibody, as described herein. For example, the anti-HIV gp120 V3 glycan binding antibody and the anti-HIV gp120 CD4bs binding antibody, as described herein, may be administered simultaneously or within seconds, minutes, hours or days of the administration of each other. In some embodiments, unit doses of an anti-HIV gp120 V3 glycan binding antibodies and anti-HIV gp120 CD4bs binding antibody disclosed herein are administered within hours of each other (e.g., within 1-12 hours, 1-24 hours, 1-36 hours, 1-48 hours, 1-60 hours, 1-72 hours).

In certain embodiments, the anti-HIV gp120 V3 glycan binding antibody and the anti-HIV gp120 CD4bs binding antibody, as described herein, are combined in a unitary dosage form, separately or as a mixture, for simultaneous administration to a patient, for example as a liquid or suspension dosage form for intravenous, intramuscular or subcutaneous administration.

In certain embodiments, the anti-HIV gp120 V3 glycan binding antibody and the anti-HIV gp120 CD4bs binding antibody are formulated, separately or as a mixture, as a liquid solution or suspension which may optionally contain one or more other agents useful for treating HIV (e.g., an HIV capsid inhibitor, e.g., lenacapavir). In certain embodiments, the liquid solution or suspension can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, such liquid solutions or suspensions are suitable for administration twice annually, e.g., once every six months (i.e., Q6M), once every 24 weeks (i.e., Q24W), once every 25 weeks (i.e., Q25W), once every 26 weeks (i.e., Q26W).

In some embodiments, after one or more co-administrations of 10-1074-LS and 3BNC117-LS, the serum concentration of 10-1074-LS and 3BNC117-LS is at least 10 μg/mL at 26 weeks after the first time point, or at 26 weeks after the most recent co-administration.

In some embodiments, after one or more co-administrations of 10-1074-LS and 3BNC117-LS, the serum concentration of HIV RNA is less than 50 copies/mL at 26 weeks after the first time point, or at 26 weeks after the most recent co-administration.

4. Patient Selection

### Stage of Infection

In various embodiments, the human subject is an adult, a juvenile or an infant. The subject may be symptomatic (e.g., viremic) or asymptomatic (e.g., acutely infected or ART suppressed). In some embodiments, the human subject is acutely infected or recently infected with HIV. In certain embodiments, the subject has not seroconverted. In some embodiments, the human subject is chronically infected with HIV. The subject may or may not be receiving a regimen of antiretroviral therapy (ART).

Patients can be categorized into Fiebig stages I-VI, which are based on a sequential gain in positive HIV-1 clinical diagnostic assays (viral RNA measured by PCR, p24 and p31 viral antigens measured by enzyme-linked immunosorbent assay (ELISA). p24 antigen is a viral core protein that transiently appears in the blood during the ramp-up phase once HIV-1 RNA levels rise above 10,000 copies/mL and before the development of detectable HIV antibodies. In Fiebig stage I, during ramp-up viremia, only HIV-1 RNA in the blood can be detected. Fiebig stage II commences about 7 days later, when results of tests to detect p24 antigen become positive. In Fiebig stage III, within about 5 days after p24 antigen test results become positive, IgM anti-HIV-1 antibodies can be detected with sufficiently sensitive enzyme immunoassays (EIAs) (e.g., third-generation EIAs). Stage III typically occurs 1-2 weeks after the onset of acute retroviral symptoms. Fiebig stage IV represents the development of an indeterminate Western blot test and occurs about 3 days after EIA tests show positive results. Conversion to a clearly positive Western blot test, Fiebig stage V, generally occurs after another 7 days, or about 1 month after initial infection. Fiebig stages of HIV infection are described, e.g., in Fiebig, et al., AIDS. (2003) 17(13):1871-9; Cohen, et al., J Infect Dis. (2010) 202 Suppl 2:S270-7; and McMichael, et al., Nature Reviews Immunology (2010) 10:11-23, which are hereby incorporated herein by reference in their entireties for all purposes. In some embodiments, the biological sample evaluated is from a human subject having an HIV infection of Fiebig stage IV or earlier, e.g., Fiebig stage I, Fiebig stage II, Fiebig stage III or Fiebig stage IV. In some embodiments, the biological sample evaluated is from a human subject having an HIV infection of an HIV infection of Fiebig stage V or Fiebig stage VI.

Sensitivity of HIV in Subject to One or Both bNAbs

In some embodiments, the methods further comprise the step of obtaining the biological sample (e.g., blood, serum, plasma, semen, lymph node) from the subject. In some embodiments, the methods entail receiving a report of the HIV gp120 amino acids residues present at the designated positions of interest, e.g., at 332 and 325, and one or more amino acid positions from the group consisting of: 63, 179, 320 and 330, wherein the amino acid positions are with reference to SEQ ID NO: 4.

In various embodiments, the methods additionally comprise the step of identifying patients most likely to benefit from therapy with one or both of the antibody targeting the V3 glycan region of HIV gp120 and the antibody targeting the CD4bs of HIV gp120. In some embodiments, sensitivity of a subject to one or both the antibody targeting the V3 glycan region of HIV gp120 and the antibody targeting the CD4bs of HIV gp120 is determined as IC90 of the bNAb is less than or equal to (≤) 2 μg/mL in PhenoSense mAb assay (Monogram).

HIV Sensitive to Anti-HIV gp120 V3-Glycan Antibodies

In some embodiments, the patient is identified by receiving a report of the HIV species infecting the patient that identifies the HIV gp120 amino acids residues present at the designated amino acid positions of interest, e.g., at positions 332 and 325, and one or more amino acid positions from the group consisting of: 63, 179, 320 and 330, wherein the amino acid positions are with reference to SEQ ID NO: 4 (supra, HXB2 subtype B HIV-1 isolate (GenBank Accession No. K0345; corresponding to residues 1-511 of NCBI Ref Seq No. NP_057856.1). Assays useful for determining whether a subject is likely to be sensitive to an anti-HIV gp120 V3-glycan antibody, including 10-1074-LS, is described, e.g., in WO 2020/236753, which is hereby incorporated herein by reference in its entirety for all purposes.

In some embodiments, the patient is identified by conducting one or more assays (e.g., polynucleotide or polypeptide sequencing) to determine the amino acid sequence(s) of the gp120 or the amino acid residues present at the designated amino acid positions of interest of the gp120 protein(s) of the HIV species infecting the patient. Identification of the full length or partial sequences of the gp120 proteins obtained from the subject can be determined at the polynucleotide or polypeptide level. In some embodiments, the amino acids present at the gp120 residue positions of interest are determined at the polypeptide level.

In various embodiments, the methods entail identifying a subject infected with an HIV or a population of HIV expressing a gp120 comprising N332glycan, D325 and T63, wherein the amino acid positions are with reference to SEQ ID NO: 4.

In various embodiments, the methods entail identifying a subject infected with an HIV or a population of HIV expressing a gp120 comprising N332glycan, D325 and L179, wherein the amino acid positions are with reference to SEQ ID NO: 4.

In various embodiments, the methods entail identifying a subject infected with an HIV or a population of HIV expressing a gp120 comprising N332glycan, D325 and T320, wherein the amino acid positions are with reference to SEQ ID NO: 4.

In various embodiments, the methods entail identifying a subject infected with an HIV or a population of HIV expressing a gp120 comprising N332glycan, D325 and H330, wherein the amino acid positions are with reference to SEQ ID NO: 4.

In various embodiments, the methods entail identifying a subject infected with an HIV or a population of HIV expressing a gp120 comprising N332glycan, D325, T63 and L179, wherein the amino acid positions are with reference to SEQ ID NO: 4.

In various embodiments, the methods entail identifying a subject infected with an HIV or a population of HIV expressing a gp120 comprising N332glycan, D325, T63 and T320, wherein the amino acid positions are with reference to SEQ ID NO: 4.

In some embodiments, the subject is infected with HIV clade B viruses. In various embodiments, the methods entail identifying a subject infected with an HIV or a population of HIV expressing a gp120 comprising N332glycan, D325, T63 and H330, wherein the amino acid positions are with reference to SEQ ID NO: 4. In various embodiments, the methods entail identifying a subject infected with an HIV or a population of HIV expressing a gp120 comprising N332glycan, D325, T63, L179, T320 and H330, wherein the amino acid positions are with reference to SEQ ID NO: 4.

In various embodiments, the methods entail identifying a subject infected with an HIV or a population of HIV expressing a gp120 comprising N332glycan, D325, T320 and H330, wherein the amino acid positions are with reference to SEQ ID NO: 4.

In various embodiments, the methods entail identifying a subject infected with an HIV or a population of HIV expressing a gp120 comprising N332glycan, D325, L179, T320 and H330, wherein the amino acid positions are with reference to SEQ ID NO: 4. In some embodiments, the subject is infected with HIV clade A and/or HIV clade C viruses. In some embodiments, the subject is infected with HIV clade A, clade B and/or HIV clade C viruses.

In various embodiments, the methods entail identifying a subject infected with an HIV or a population of HIV expressing a gp120 comprising N332glycan, D325, T63, L179 and T320, wherein the amino acid positions are with reference to SEQ ID NO: 4.

In various embodiments, the methods entail identifying a subject infected with an HIV or a population of HIV expressing a gp120 comprising N332glycan, D325, T63, L179 and H330, wherein the amino acid positions are with reference to SEQ ID NO: 4.

HIV Sensitive to Anti-HIV gp120 CD4bs Antibodies

In some embodiments, the patient is identified by receiving a report of the HIV species infecting the patient that identifies the HIV gp120 amino acids residues present at the designated amino acid positions of interest, e.g., at position 201, and one or more amino acid positions from the group consisting of: 102, 108, 281, 318 and 353, wherein the amino acid positions are with reference to SEQ ID NO: 439. In some embodiments, the patient is identified by conducting one or more assays (e.g., polynucleotide or polypeptide sequencing) to determine the amino acid sequence(s) of the gp120 or the amino acid residues present at the designated amino acid positions of interest of the gp120 protein(s) of the HIV species infecting the patient. Identification of the full length or partial sequences of the gp120 proteins obtained from the subject can be determined at the polynucleotide or polypeptide level. In some embodiments, the amino acids present at the gp120 residue positions of interest are determined at the polypeptide level. Assays useful for determining whether a subject is likely to be sensitive to an anti-HIV gp120 CD4 binding site antibody, including 3BNC117-LS, is described, e.g., in WO 2022/103758, which is hereby incorporated herein by reference in its entirety for all purposes.

In various embodiments, the methods entail identifying a subject infected with an HIV or a population of HIV expressing a gp120 comprising I201 and F353, wherein the amino acid positions are with reference to SEQ ID NO: 439.

In various embodiments, the methods entail identifying a subject infected with an HIV or a population of HIV expressing a gp120 comprising I201, I108 and F353, wherein the amino acid positions are with reference to SEQ ID NO: 439.

In various embodiments, the methods entail identifying a subject infected with an HIV or a population of HIV expressing a gp120 comprising I201, I108, A281 and F353, wherein the amino acid positions are with reference to SEQ ID NO: 439.

In various embodiments, the methods entail identifying a subject infected with an HIV or a population of HIV expressing a gp120 comprising I201, E102, I108, A281 and F353, wherein the amino acid positions are with reference to SEQ ID NO: 439.

In various embodiments, the methods entail identifying a subject infected with an HIV or a population of HIV expressing a gp120 comprising I201, E102, I108, A281, Y318 and F353, wherein the amino acid positions are with reference to SEQ ID NO: 439.

In some embodiments, the subject is infected with HIV clade (a.k.a., HIV subtype) B viruses. In some embodiments, the subject is infected with HIV clade (a.k.a., HIV subtype) A and/or HIV clade (a.k.a., HIV subtype) C viruses. In some embodiments, the subject is infected with HIV clade (a.k.a., HIV subtype) A, clade B and/or HIV clade (a.k.a., HIV subtype) C viruses.

Determining gp120 Amino Acids of Interest

Determination of the amino acid residues at HIV gp120 sequences of a subject at the designated positions of interest, e.g., at 332 and 325, and one or more amino acid positions from the group consisting of: 63, 179, 320 and 330, wherein the amino acid positions are with reference to SEQ ID NO: 3, can be done at the polynucleotide or polypeptide level. At the level of the polynucleotide, HIV RNA or proviral DNA isolated from one or more biological samples can be sequenced using methods known in the art. In some embodiments, HIV RNA or proviral DNA isolated from two or more biological samples of a subject are sequenced. In some embodiments, the two or more biological samples are obtained from different tissue sources (e.g., blood, peripheral blood mononuclear cells, lymph nodes and/or semen). In some embodiments, the two or more biological samples are obtained at different time points, e.g., 1, 2, 3, 4, 5, 6, 7 or 8 weeks apart, 3, 4, 5, 6, 7, 8, 9, 10, 11 or 12 months apart.

As appropriate, primers that anneal to and amplify the HIV env coding sequence, and particularly the CD4bs region of gp120, can be used. In some embodiments, nested sets of primers can be used. In various embodiments, the RNA is sequenced directly or reverse-transcriptase polymerase chain reaction (RT-PCR) can be performed. In some embodiments, Sanger sequencing can be performed, e.g., when sequencing to determine amino acid residues in the CD4bs region, or when sequencing a sample from a patient in an early Fiebig stage of disease, e.g., prior to Fiebig stage III, e.g., Fiebig stages I or II. In various embodiments, single genome amplification (SGA) and sequencing is performed. Methods for single genome amplification (SGA) and sequencing of plasma HIV virion RNA, are described, e.g., in Salazar-Gonzalez, et al. (2008) J Virol 82:3952-3970; and Keele, et al., Proc Natl Acad Sci USA. (2008) 105(21):7552-7. Application of SGA to determining amino acid sequence variance in HIV gp120 sequences, and which can be employed in the herein described methods, is described, e.g., in Bar, et al., N Engl J Med. (2016) 375(21):2037-2050; and Mendoza, et al., Nature. (2018) 561(7724):479-484. In various embodiments, high throughput, Next Generation Sequencing (NGS), massively parallel or deep sequencing techniques are employed to sequence gp120, including at least the CD4bs region, from a population of HIV species in one or more biological samples from a single patient or subject. In such cases, multiple nucleic acid sequences encoding at least the CD4bs region of gp120 are sequenced and aligned. In some embodiments, the full-length of gp120 is sequenced. Illustrative platforms for performing NGS sequencing that can be used for determining the gp120 sequences of HIV species in one or more biological samples from a patient include Illumina (Solexa) (illumina.com), Ion torrent: Proton/PGM sequencing (thermofisher.com), SOLiD (thermofisher.com), and Single Molecule, Real-Time (SMRT) Sequencing (Pacific Biosciences, pacb.com). Methods for isolating and sequencing HIV gp120, including at least the CD4bs region, from patients, and which can be applied in the present methods, are described in, e.g., Shioda, et al., J Virol. (1997) 71(7):4871-81; Colón, et al., J Virol Antivir Res. (2015) 4(3). pii: 143 (PMID: 27358904); Kafando, et al., PLoS One. (2017) 12(12):e0189999; Hebberecht, et al., PLoS One. (2018) 13(4):e0195679, Andrews, et al., Sci Rep. (2018) 8(1):5743 and Landais, et al. Immunity. (2017) 47(5):990-1003. As appropriate, shorter sequence reads of the nucleic acid sequences (“contigs”) can be assembled into longer sequences, including at least the CD4bs region of gp120. Methods of contig assembly of HIV genomic sequences that can be applied in the present methods are described, e.g., in Huang, et al., Bioinformation. (2018) 14(8):449-454; Hiener, et al., J Vis Exp. (2018) Oct. 16; (140). doi: 10.3791/58016; and Wymant, et al., Virus Evol. (2018) May 18; 4(1):vey007. doi: 10.1093/ve/vey007.

5. Combination Therapies

In certain embodiments, a method for treating or preventing an HIV infection in a human having or at risk of having the infection is provided, comprising administering to the human a therapeutically effective amount of the anti-HIV gp120 V3 glycan and anti-HIV gp120 CD4bs binding antibodies, as disclosed herein, in combination with a therapeutically effective amount of one or more (e.g., one, two, three, four, one or two, one to three or one to four) additional therapeutic agents. In one embodiment, a method for treating an HIV infection in a human having or at risk of having the infection is provided, comprising administering to the human a therapeutically effective amount of the anti-HIV gp120 V3 glycan and anti-HIV gp120 CD4bs binding antibodies, as disclosed herein, in combination with a therapeutically effective amount of one or more (e.g., one, two, three, four, one or two, one to three or one to four) additional therapeutic agents.

In one embodiment, pharmaceutical compositions comprising the anti-HIV gp120 V3 glycan and anti-HIV gp120 CD4bs binding antibodies, as disclosed herein, in combination with one or more (e.g., one, two, three, four, one or two, one to three or one to four) additional therapeutic agents, and a pharmaceutically acceptable carrier, diluent, or excipient are provided.

In certain embodiments, provided are methods for treating an HIV infection, comprising administering to a patient in need thereof a therapeutically effective amount of the anti-HIV gp120 V3 glycan and anti-HIV gp120 CD4bs binding antibodies or antigen-binding fragment thereof, as described herein, in combination with a therapeutically effective amount of one or more additional therapeutic agents which are suitable for treating an HIV infection.

In certain embodiments, the anti-HIV gp120 V3 glycan and anti-HIV gp120 CD4bs binding antibodies or antigen-binding fragment thereof is combined with one, two, three, four, or more additional therapeutic agents. In certain embodiments, the anti-HIV gp120 V3 glycan and anti-HIV gp120 CD4bs binding antibodies or antigen-binding fragment thereof is combined with two additional therapeutic agents. In other embodiments, the anti-HIV gp120 V3 glycan and anti-HIV gp120 CD4bs binding antibodies or antigen-binding fragment thereof is combined with three additional therapeutic agents. In further embodiments, the anti-HIV gp120 V3 glycan and anti-HIV gp120 CD4bs binding antibodies or antigen-binding fragment thereof is combined with four additional therapeutic agents. The one, two, three, four, or more additional therapeutic agents can be different therapeutic agents selected from the same class of therapeutic agents, (e.g., one or more anti-HIV broadly neutralizing antibodies), and/or they can be selected from different classes of therapeutic agents.

### Administration of HIV Combination Therapy

In certain embodiments, the anti-HIV gp120 V3 glycan and anti-HIV gp120 CD4bs binding antibodies or antigen-binding fragment thereof, as described herein, is co-administered with one or more additional therapeutic agents. Co-administration of an anti-HIV gp120 CD4bs binding antibodies disclosed herein with one or more additional therapeutic agents generally refers to simultaneous or sequential administration of an anti-HIV gp120 CD4bs binding antibodies disclosed herein and one or more additional therapeutic agents, such that therapeutically effective amounts of the anti-HIV gp120 V3 glycan and anti-HIV gp120 CD4bs binding antibodies disclosed herein and the one or more additional therapeutic agents are both present in the body of the patient. When administered sequentially, the combination may be administered in two or more administrations.

Co-administration includes concurrent administration as well as administration of unit dosages of the anti-HIV gp120 V3 glycan and anti-HIV gp120 CD4bs binding antibodies or antigen-binding fragment thereof, as described herein before or after administration of unit dosages of one or more additional therapeutic agents. For example, the anti-HIV gp120 V3 glycan and anti-HIV gp120 CD4bs binding antibodies or antigen-binding fragment thereof, as described herein, may be administered within seconds, minutes, hours or days of the administration of the one or more additional therapeutic agents. In some embodiments, a unit doses of anti-HIV gp120 V3 glycan and anti-HIV gp120 CD4bs binding antibodies disclosed herein is administered first, followed within seconds, minutes, hours or days by administration of a unit dose of one or more additional therapeutic agents. Alternatively, a unit dose of one or more additional therapeutic agents is administered first, followed by administration of a unit doses anti-HIV gp120 V3 glycan and anti-HIV gp120 CD4bs binding antibodies disclosed herein within seconds, minutes, hours or days. In other embodiments, a unit doses of anti-HIV gp120 V3 glycan and anti-HIV gp120 CD4bs binding antibodies disclosed herein is administered first, followed, after a period of hours (e.g., 1-12 hours, 1-24 hours, 1-36 hours, 1-48 hours, 1-60 hours, 1-72 hours), by administration of a unit dose of one or more additional therapeutic agents. In yet other embodiments, a unit dose of one or more additional therapeutic agents is administered first, followed, after a period of hours (e.g., 1-12 hours, 1-24 hours, 1-36 hours, 1-48 hours, 1-60 hours, 1-72 hours), by administration of a unit dose of an anti-HIV gp120 CD4bs binding antibodies disclosed herein.

In certain embodiments, the anti-HIV gp120 V3-glycan binding antibody and the anti-HIV gp120 CD4bs binding antibody disclosed herein are further combined with one or more additional therapeutic agents in a unitary dosage form for simultaneous administration to a patient, for example as a solid, liquid or suspension dosage form for oral, intravenous, intramuscular or subcutaneous administration.

In certain embodiments, the serum half-life extended anti-HIV gp120 V3 glycan and anti-HIV gp120 CD4bs binding antibodies are formulated as a liquid solution or suspension which may optionally contain one or more other compounds useful for treating HIV. In certain embodiments, the liquid solution or suspension can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, such liquid solutions or suspensions are suitable for administration twice annually, e.g., every 6 months (Q6M), every 26 weeks (Q26W), every 25 weeks (Q25W), or every 24 weeks (Q24W).

### HIV Combination Therapy

In the above embodiments, the additional therapeutic agent may be an anti-HIV agent. Illustrative anti-HIV agents that can be combined or co-administered include without limitation a third anti-HIV antibody, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators (e.g., immunostimulators), immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, prolylendopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, HIV capsid inhibitors, HIV Tat or Rev inhibitors, immunomodulators, (e.g., immunostimulators), immunotherapeutic agents, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and/or TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof.

### Additional Anti-HIV Antibodies

In some embodiments, the anti-HIV gp120 V3-glycan binding antibody and the anti-HIV gp120 CD4bs binding antibody disclosed herein are further combined with one or more additional anti-HIV antibodies. In some embodiments, the one or more additional antibodies bind to an epitope or region of gp120 selected from the group consisting of: (i) second variable loop (V2) and/or Env trimer apex; (ii) gp120/gp4l interface; or (iii) silent face of gp120. The foregoing epitopes or regions of gp120 bound by broadly neutralizing antibodies are described, e.g., in McCoy, Retrovirology (2018) 15:70; Sok and Burton, Nat Immunol. 2018 19(11):1179-1188; Possas, et al., Expert Opin Ther Pat. 2018 July; 28(7):551-560; and Stephenson and Barouch, Curr HIV/AIDS Rep (2016) 13:31-37, which are hereby incorporated herein by reference in their entirety for all purposes.

In some embodiments, the combination therapy entails co-administration of an anti-HIV gp120 V3-glycan binding antibody and the anti-HIV gp120 CD4bs binding antibody and one or more additional anti-HIV broadly neutralizing antibodies or bNAbs (i.e., a neutralizing antibody that neutralizes multiple HIV-1 viral strains). Various bNAbs are known in the art and may be used as a combining therapeutic agent. Additional illustrative bNAbs of use include, those that comprise VH and VL that bind to or compete with an epitope or region of gp120 selected from the group consisting of: (i) second variable loop (V2) and/or Env trimer apex; (ii) gp120/gp41 interface; or (iii) silent face of gp120.

In some embodiments, the combination therapy includes an antibody that binds to an epitope or region of gp120 in the second variable loop (V2) and/or Env trimer apex and competes with or comprises CDRs and/or VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01.

In some embodiments, the combination therapy includes an antibody that binds to an epitope or region of gp120 in the gp120/gp41 interface and competes with or comprises CDRs and/or VH and VL regions from an antibody selected from the group consisting of PGT-151, CAP248-2B, 35O22, 8ANC195, ACS202, VRC34 and VRC34.01.

In some embodiments, the combination therapy includes an antibody that binds to an epitope or region of the gp120 silent face and competes with or comprises second VH and VL regions from antibody VRC-PG05.

In some embodiments, the combination therapy includes an antibody that binds to an epitope or region of gp41 in the membrane proximal region (MPER) and competes with or comprises second VH and VL regions from an antibody selected from the group consisting of 10E8, 10E8v4, 10E8-5R-100cF, 4E10, DH511.11P, 2F5, 7b2, and LN01. In some embodiments, the combination therapy includes an antibody that binds to an epitope or region of KLIC (“KLIC” disclosed as SEQ ID NO: 496), an immutable site of the transmembrane protein gp41 and competes with or comprises second VH and VL regions from Clone 3 human monoclonal antibody (Cl3hmAb) (Protheragen). See, e.g., Vanini, et al., AIDS. (1993) 7(2):167-74.

In some embodiments, the combination therapy includes an antibody that binds to and epitope or region of the gp41 fusion peptide and competes with or comprises second VH and VL regions from an antibody selected from the group consisting of VRC34 and ACS202.

In some embodiments, the combination therapy includes a multi-specific, e.g., a bispecific or tri-specific antibody that binds to an HIV antigen. Examples of HIV bispecific and trispecific antibodies include MGD014, B12BiTe, BiIA-SG, TMB-bispecific, SAR-441236, VRC-01/PGDM-1400/10E8v4, 10E8.4/iMab, and 10E8v4/PGT121-VRC01.

Prior to administration, the bNAbs may be improved to have enhanced drug-like-properties, reduced immunogenicity, enhanced ADCC, and suitable pharmacokinetic properties. Such antibodies were shown to bind to the HIV envelope glycoprotein expressed on the surface of virion or infected cells, and mediate both direct neutralization of the virus as well as potent NK, Monocyte and PBMC killing of these cells. This property allows the antibodies to treat HIV infections by neutralizing the virus, and also kill and eliminate latently HIV infected cells in infected individuals, potentially leading to a sterilizing cure for HIV.

In various embodiments, all antibodies administered in a combination anti-HIV antibody therapy can have Fc and/or post-translational modifications that increase serum half-life and/or enhance effector activity, as described above.

In various embodiments, the anti-HIV gp120 CD4bs binding antibody or antigen-binding fragments, and optionally combined bNAbs, can be in vivo delivered, e.g., expressed in vivo from administered mRNA or engineered B-cells. Examples of in vivo delivered bNAbs include AAV8-VRC07; mRNA encoding anti-HIV antibody VRC01; and engineered B-cells encoding 3BNC117 (Hartweger et al, J. Exp. Med. 2019, 1301).

### HIV Combination Drugs

In certain embodiments, the anti-HIV gp120 V3 glycan and anti-HIV gp120 CD4bs binding antibodies described herein are combined with one, two, three, four or more additional anti-HIV therapeutic agents. Example anti-HIV combination drugs that can be co-administered include without limitation ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir and lamivudine), TRIZIVIR® (abacavir sulfate, zidovudine, and lamivudine; ABC+AZT+3TC); atazanavir and ritonavir (ATZ+RTV); atazanavir and cobicistat; atazanavir sulfate and cobicistat; atazanavir sulfate and ritonavir; PREZCOBIX® (darunavir and cobicistat); dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, ACC-008 (ACC-007+lamivudine+tenofovir disoproxil fumarate), VM-1500+emtricitabine+tenofovir disoproxil, and tenofovir disoproxil fumarate+emtricitabine+rilpivirine hydrochloride, lopinavir, ritonavir, zidovudine, lopinavir+ritonavir+abacavir+lamivudine, and lamivudine; cabotegravir+rilpivirine; 3-BNC117+albuvirtide, (elsulfavirine; VM-1500), VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, Gamimune, metenkefalin, naltrexone, Prolastin, REP 9, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat, ABBV-382, obefazimod (ABX-464), AG-1105, APH-0812, APH0202, bryostatin-1, bryostatin-23, bryostatin analogs, SUW-133, BIT-225, BRII-732, BRII-778, Codivir, CYT-107, CS-TATI-1, fluoro-beta-D-arabinose nucleic acid (FANA)-modified antisense oligonucleotides, FX-101, griffithsin, HGTV-43, HPH-116, HRS-5685, HivCide-I, hydroxychloroquine, IMB-10035, IMO-3100, IND-02, JL-18008, LADAVRU, LLDT-8, MK-1376, MK-2048, MK-4250, MK-8507, MK-8558, islatravir (MK-8591), NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, T-1144, VIR-576, nipamovir, Covimro, WP-1122, ZFP-362, and ABBV-1882.

### HIV Protease Inhibitors

In certain embodiments, the anti-HIV gp120 V3 glycan and anti-HIV gp120 CD4bs binding antibodies described herein are combined with an HIV protease inhibitor. Examples of HIV protease inhibitors include without limitation amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, elunonavir (GS-1156), TMB-657 (PPL-100), T-169, BL-008, MK-8122, TMB-607, GRL-02031 and TMC-310911. Additional examples of HIV protease inhibitors are described, e.g., in U.S. Pat. No. 10,294,234, and U.S. Patent Appl. Publ. Nos. US2020030327 and US2019210978.

### HIV Ribonuclease H Inhibitors

In certain embodiments, the anti-HIV gp120 V3 glycan and anti-HIV gp120 CD4bs binding antibodies described herein are combined with an HIV ribonuclease H inhibitor. Examples of HIV ribonuclease H inhibitors that can be combined include without limitation NSC-727447.

### HIV Nef Inhibitors

In certain embodiments, the anti-HIV gp120 V3 glycan and anti-HIV gp120 CD4bs binding antibodies described herein are combined with an HIV Nef inhibitor. Examples of HIV Nef inhibitors that can be combined with include without limitation FP-1.

### HIV Reverse Transcriptase Inhibitors

In certain embodiments, the anti-HIV gp120 V3 glycan and anti-HIV gp120 CD4bs binding antibodies described herein are combined with a non-nucleoside or non-nucleotide inhibitor. Examples of HIV non-nucleoside or non-nucleotide inhibitors of reverse transcriptase include without limitation dapivirine, delavirdine, delavirdine mesylate, doravirine, difluoro-biphenyl-diarylpyrimidines (DAPY), efavirenz, etravirine, GS-5894, lentinan, nevirapine, rilpivirine, ACC-007, ACC-018, AIC-292, F-18, KM-023, PC-1005, M1-TFV, M2-TFV, VM-1500A-LAI, PF-3450074, elsulfavirine (sustained release oral), doravirine+islatravir (fixed dose combination/oral tablet formulation), elsulfavirine (long acting injectable nanosuspension), and elsulfavirine (VM-1500).

In certain embodiments, the anti-HIV gp120 V3 glycan and anti-HIV gp120 CD4bs binding antibodies described herein are combined with an HIV nucleoside or nucleotide inhibitor. Examples of HIV nucleoside or nucleotide inhibitors of reverse transcriptase include without limitation adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir amibufenamide fumarate (HS-10234), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, GS-1614, GSK-4023991, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

In certain embodiments, the anti-HIV gp120 V3 glycan and anti-HIV gp120 CD4bs binding antibodies described herein are combined with an HIV integrase inhibitor. Examples of HIV integrase inhibitors include without limitation elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, GS-1720, GS-6212, GS-1219, GS-3242, VH4524184, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, S-365598, stilbenedisulfonic acid, T169, STP-0404, VM-3500, XVIR-110, and ACC-017.

In certain embodiments, the anti-HIV gp120 V3 glycan and anti-HIV gp120 CD4bs binding antibodies described herein are combined with a HIV non-catalytic site, or allosteric, integrase inhibitor (NCINI). Examples of HIV non-catalytic site, or allosteric, integrase inhibitors (NCINI) include without limitation CX-05045, CX-05168, and CX-14442.

### Capsid Inhibitors

In certain embodiments, the anti-HIV gp120 V3 glycan and anti-HIV gp120 CD4bs binding antibodies described herein are combined with a capsid inhibitor. Examples of capsid inhibitors that can be combined with an agent of this disclosure include capsid polymerization inhibitors or capsid disrupting compounds, HIV nucleocapsid p7 (NCp7) inhibitors such as azodicarbonamide, HIV p24 capsid protein inhibitors, lenacapavir (GS-6207), VH4004280, VH4011499, GS-CA1, AVI-621, AVI-101, AVI-201, AVI-301, and AVI-CAN1-15 series, PF-3450074, and compounds described in Intl. Patent Publ. No. WO 2019/087016 and U.S. Patent Publ. Nos. US2014/0221356, US2016/0016973, US2018/0051005, US2016/0108030.

### HIV Viral Infectivity Factor Inhibitors

In certain embodiments, the anti-HIV gp120 V3 glycan and anti-HIV gp120 CD4bs binding antibodies described herein are combined with an HIV viral infectivity factor inhibitor. Examples of HIV viral infectivity factor inhibitors include 2-amino-N-(2-methoxyphenyl)-6-((4-nitrophenyl)thio)benzamide derivatives and Irino-L.

### HIV Entry Inhibitors

In certain embodiments, the anti-HIV gp120 V3 glycan and anti-HIV gp120 CD4bs binding antibodies described herein are combined with an HIV entry inhibitor. Examples of HIV entry (fusion) inhibitors include AAR-501, LBT-5001, cenicriviroc, CCR5 inhibitors, gp41 inhibitors, CD4 attachment inhibitors, gp120 inhibitors, gp160 inhibitors and CXCR4 inhibitors.

In certain embodiments, the anti-HIV gp120 V3 glycan and anti-HIV gp120 CD4bs binding antibodies described herein are combined with a CCR5 inhibitor. Examples of CCR5 inhibitors include aplaviroc, vicriviroc, maraviroc, maraviroc (long-acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

In certain embodiments, the anti-HIV gp120 V3 glycan and anti-HIV gp120 CD4bs binding antibodies described herein are combined with a CXCR4 inhibitor. Examples of CXCR4 inhibitors include plerixafor, ALT-1188, N15 peptide, balixafortide and vMIP (Haimipu).

In certain embodiments, the anti-HIV gp120 V3 glycan and anti-HIV gp120 CD4bs binding antibodies described herein are combined with a gp41 inhibitor. Examples of gp41 inhibitors include albuvirtide, enfuvirtide, griffithsin (gp41/gp120/gp160 inhibitor), BMS-986197, HIV-1 fusion inhibitors (P26-Bapc), ITV-1, ITV-2, ITV-3, ITV-4, CPT-31, Cl3hmAb, lipovirtide, PIE-12 trimer and sifuvirtide.

In certain embodiments, the anti-HIV gp120 V3 glycan and anti-HIV gp120 CD4bs binding antibodies described herein are combined with a CD4 attachment inhibitor. Examples of CD4 attachment inhibitors include ibalizumab and CADA analogs.

In certain embodiments, the anti-HIV gp120 V3 glycan and anti-HIV gp120 CD4bs binding antibodies described herein are combined with a gp120 inhibitor. Examples of gp120 inhibitors include anti-HIV microbicide, Radha-108 (receptol) 3B3-PE38, BMS818251, BanLec, bentonite-based nanomedicine, fostemsavir tromethamine, IQP-0831, VVX-004, and BMS-663068.

In certain embodiments, the anti-HIV gp120 V3 glycan and anti-HIV gp120 CD4bs binding antibodies described herein are combined with a gp160 inhibitor. Examples of gp160 inhibitors that can be combined include fangchinoline.

### HIV Maturation Inhibitors

In certain embodiments, the anti-HIV gp120 V3 glycan and anti-HIV gp120 CD4bs binding antibodies described herein are combined with an HIV maturation inhibitor. Examples of HIV maturation inhibitors include BMS-955176, GSK-3640254, VH-3739937 (GSK-3739937), HRF-10071 and GSK-2838232.

### Latency Reversing Agents

In certain embodiments, the anti-HIV gp120 V3 glycan and anti-HIV gp120 CD4bs binding antibodies described herein are combined with an HIV latency reversing agent. Examples of latency reversing agents that can be combined with the one or more multi-specific antigen binding molecules, described herein, include IL-15 receptor agonists (e.g., ALT-803; interleukin-15/Fc fusion protein (e.g., XmAb24306); recombinant interleukin-15 (e.g., AM0015, NIZ-985); pegylated IL-15 (e.g., NKTR-255)); toll-like receptor (TLR) agonists (including TLR7 agonists, e.g., vesatolimod (GS-9620); TLR8 agonists, e.g., selgantolimod (GS-9688); TLR9 agonists, e.g., lefitolimod (MGN-1703), histone deacetylase (HDAC) inhibitors, proteasome inhibitors such as velcade, protein kinase C (PKC) activators, Smyd2 inhibitors, BET-bromodomain 4 (BRD4) inhibitors (e.g., such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, xevinapant (AT-406)), Debio-1143, PMA, SAHA (suberanilohydroxamic acid, or suberoyl, anilide, and hydroxamic acid), NIZ-985, IL-15 modulating antibodies, (including IL-15, IL-15 fusion proteins and IL-15 receptor agonists, e.g., ALT-803), JQ1, disulfiram, amphotericin B, and ubiquitin inhibitors such as largazole analogs, APH-0812, and GSK-343. Examples of PKC activators include indolactam, prostratin, ingenol B, and DAG-lactones.

### Toll-Like Receptor (TLR) Agonists

In certain embodiments, the anti-HIV gp120 V3 glycan and anti-HIV gp120 CD4bs binding antibodies described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793).

Example TLR7 agonists that can be co-administered or combined with the one or more multi-specific antigen binding molecules, described herein, include without limitation AL-034, DSP-0509, GS-9620 (vesatolimod), vesatolimod analogs, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), US20090047249 (Gilead Sciences), US2010143301 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics).

An TLR7/TLR8 agonist that can be co-administered is NKTR-262, telratolimod and BDB-001.

Example TLR8 agonists that can be co-administered or combined with the one or more multi-specific antigen binding molecules, described herein, include without limitation E-6887, IMO-4200, IMO-8400, IMO-9200, MCT-465, MEDI-9197, motolimod, resiquimod, selgantolimod (GS-9688), VTX-1463, VTX-763, 3M-051, 3M-052, and the compounds disclosed in US2017071944 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics).

Example TLR9 agonists that can be co-administered include without limitation AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. Examples of TLR4 agonist include G-100, and GSK-1795091.

### Histone Deacetylase (HDAC) Inhibitors

In certain embodiments, the anti-HIV gp120 V3 glycan and anti-HIV gp120 CD4bs binding antibodies described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Cytochrome P450 3 Inhibitors

In certain embodiments, the anti-HIV gp120 V3 glycan and anti-HIV gp120 CD4bs binding antibodies described herein are combined with a cytochrome P450 3 inhibitor. Examples of Cytochrome P450 3 inhibitors include without limitation those described in U.S. Pat. No. 7,939,553.

### RNA Polymerase Modulators

In certain embodiments, the anti-HIV gp120 V3 glycan and anti-HIV gp120 CD4bs binding antibodies described herein are combined with an RNA polymerase modulator. Examples of RNA polymerase modulators include without limitation those described in U.S. Pat. Nos. 10,065,958 and 8,008,264.

### Cyclin-Dependent Kinase (CDK) Inhibitors or Antagonists

In certain embodiments, the anti-HIV gp120 V3 glycan and anti-HIV gp120 CD4bs binding antibodies described herein are combined with an inhibitor or antagonist of a cyclin-dependent kinase (CDK), e.g., cyclin dependent kinase 4 (CDK4; NCBI Gene ID: 1019), cyclin dependent kinase 6 (CDK6; NCBI Gene ID: 1021), cyclin dependent kinase 9 (CDK9; NCBI Gene ID: 1025). In some embodiments, the CDK4/CDK6/CDK9 inhibitor or antagonist is selected from the group consisting of VS2-370.

### Stimulator of Interferon Genes (STING) Agonists

In some embodiments, the anti-HIV gp120 V3 glycan and anti-HIV gp120 CD4bs binding antibodies described herein are combined with an stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP) and cyclic-di-AMP.

### RIG-I Agonists

In certain embodiments, the anti-HIV gp120 V3 glycan and anti-HIV gp120 CD4bs binding antibodies described herein are combined with an agonist of DExD/H-box helicase 58 (DDX58; a.k.a., RIG-I, RIG1, RIGI, RLR-1, SGMRT2; NCBI Gene ID: 23586). In some embodiments, the agents described herein are combined with a RIG-I modulator such as RGT-100, or NOD2 modulator, such as SB-9200 (a.k.a., GS 9992; inarigivir), and IR-103. An illustrative RIG-I agonist is KIN1148, described by Hemann, et al., J Immunol May 1, 2016, 196 (1 Supplement) 76.1. Additional RIG-I agonists are described, e.g., in Elion, et al., Cancer Res. (2018) 78(21):6183-6195; and Liu, et al., J Virol. (2016) 90(20):9406-19. RIG-I agonists are commercially available, e.g., from Invivogen (invivogen.com).

### LAG-3 and TIM-3 Inhibitors

In certain embodiments, the anti-HIV gp120 V3 glycan and anti-HIV gp120 CD4bs binding antibodies described herein are combined with an anti-TIM-3 (a.k.a., hepatitis A virus cellular receptor 2 antibody (HAVCR2; NCBI Gene ID: 84868), such as TSR-022, LY-3321367, MBG-453, INCAGN-2390. In some embodiments, the anti-HIV gp120 V3 glycan and anti-HIV gp120 CD4bs binding antibodies described herein are combined with an anti-LAG-3 (Lymphocyte-activation) (NCBI Gene ID: 3902) antibody, such as relatlimab (ONO-4482), LAG-525, MK-4280, REGN-3767, INCAGN2385.

### Immune-Based Therapies

In certain embodiments, the anti-HIV gp120 V3 glycan and anti-HIV gp120 CD4bs binding antibodies described herein are combined with an immune-based therapy. Examples of immune-based therapies include toll-like receptor (TLR) modulators such as TLR1, TLR2, TLR3, TLR4, TLR5, TLR6, TLR7, TLR8, TLR9, TLR10, TLR11, TLR12, AND TLR13; programmed cell death protein 1 (PD-1) modulators; programmed death-ligand 1 (PD-L1) modulators; IL-15 modulators (e.g., IL-15 receptor agonists (e.g., ALT-803; interleukin-15/Fc fusion protein (e.g., XmAb24306); recombinant interleukin-15 (e.g., AM0015, NIZ-985); pegylated IL-15 (e.g., NKTR-255)); DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; polymer polyethyleneimine (PEI); gepon; IL-12; WF-10; VGV-1; MOR-22; BMS-936559; CYT-107, normferon, peginterferon alfa-2a, peginterferon alfa-2b, RPI-MN, STING modulators, RIG-I modulators, NOD2 modulators, SB-9200, and IR-103.

In certain embodiments, the anti-HIV gp120 V3 glycan and anti-HIV gp120 CD4bs binding antibodies described herein are combined with a TLR agonist. Examples of TLR agonists include without limitation: vesatolimod (GS-9620), lefitolimod, tilsotolimod, rintatolimod, DSP-0509, AL-034, G-100, cobitolimod, AST-008, motolimod, GSK-1795091, GSK-2245035, VTX-1463, selgantolimod (GS-9688), LHC-165, BDB-001, RG-7854, telratolimod.

### Immune Checkpoint Receptor Protein Modulators

In various embodiments, the anti-HIV gp120 V3 glycan and anti-HIV gp120 CD4bs binding antibodies described herein are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688).

Examples of immune checkpoint proteins or receptors that can be combined with the anti-HIV gp120 V3 glycan and anti-HIV gp120 CD4bs binding antibodies described herein include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and Hematopoietic Progenitor Kinase 1 (HPK1, MAP4K1).

In various embodiments, the anti-HIV gp120 V3 glycan and anti-HIV gp120 CD4bs binding antibodies described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the anti-HIV gp120 V3 glycan and anti-HIV gp120 CD4bs binding antibodies described herein are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In various embodiments, the anti-HIV gp120 V3 glycan and anti-HIV gp120 CD4bs binding antibodies described herein are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the anti-HIV gp120 V3 glycan and anti-HIV gp120 CD4bs binding antibodies described herein are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of programmed cell death 1 (PDCD1; NCBI Gene ID: 5133; CD279, PD-1, PD1) that can be combined or co-administered include without limitation zimberelimab (AB122, GLS-010, WBP-3055), pembrolizumab (KEYTRUDA®, MK-3475, SCH900475), nivolumab (OPDIVO®, BMS-936558, MDX-1106), cemiplimab (LIBTAYO®; cemiplimab-rwlc, REGN-2810), pidilizumab (CT-011), AMG-404, MEDI0680 (AMP-514), spartalizumab (PDR001), tislelizumab (BGB-A317), toripalimab (JS-001), genolimzumab (CBT-501, APL-501, GB 226), SHR-1201, camrelizumab (SHR-1210), sintilimab (TYVYT®; IBI-308), dostarlimab (TSR-042, WBP-285), lambrolizumab (MK-3475); sasanlimab (PF-06801591), cetrelimab (JNJ-63723283), serplulimab (HLX-10), retifanlimab (MGA-012), balstilimab (AGEN2034), prolgolimab (BCD 100), budigalimab (ABBV-181), vopratelimab (JTX-4014), AK-103 (HX-008), AK-105, CS-1003, BI-754091, LZM-009, Sym-021, BAT-1306, PD1-PIK, tebotelimab (MGD013; PD-1/LAG-3), RO-7247669 (PD-1/LAG-3), FS-118 (LAG-3/PD-L1), RO-7121661 (PD-1/TIM-3), RG7769 (PD-1/TIM-3), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1) and MEDI-5752 (CTLA4/PD-1). In some embodiments, the first and/or second antigen binding domain comprises the extracellular domain of the human programmed cell death 1 ligand 2 (PD-L2) and binds to PD1 (e.g., AMP-224).

Examples of inhibitors of CD274 molecule (NCBI Gene ID: Gene ID: 29126; B7-H, B7H1, PD-L1) that can be combined or co-administered include without limitation atezolizumab (TECENTRIQ®), avelumab (BAVENCIO®; MSB0010718C), envafolimab (ASC22), durvalumab (IMFINZI®; MEDI-4736), BMS-936559 (MDX1105), cosibelimab (CK-301), lodapolimab (LY 3300054), garivulimab (BGB A333), envafolimab (KN035), opucolimab (HLX 20), manelimab (BCD 135), CX-072, CBT-502 (TQB2450), MSB-2311, SHR-1316, sugemalimab (CS-1001; WBP3155), A167 (KL-A167, HBM 9167), STI-A1015 (IMC-001), FAZ-053, BMS-936559 (MDX1105), INCB086550, GEN-1046 (PD-L1/4-1BB), FPT-155 (CTLA4/PD-L1/CD28), M7824 (PD-L1/TGFβ-EC domain), CA-170 (PD-L1/VISTA), CDX-527 (CD27/PD-L1), LY-3415244 (TIM-3/PDL1), INBRX-105 (4-1BB/PDL1) and GNS-1480 (PD-L1/EGFR), and further includes human-derived, allogeneic, natural killer cells engineered to express a chimeric antigen receptor (CAR) targeting PD-L1, such as PD-L1 t-haNK.

In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

In various embodiments, the antibodies as described herein are combined with anti-TIGIT antibodies, such as domvanalimab, ralzapastotug, vibostolimab, ociperlimab, tiragolumab, rilvegostomig, belrestotug, etigilimab, BMS-986207, RG-6058, or AGEN-1307.

### TNF Receptor Superfamily (TNFRSF) Member Agonists or Activators

In various embodiments, the anti-HIV gp120 V3 glycan and anti-HIV gp120 CD4bs binding antibodies described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Interleukin Receptor Agonists

In certain embodiments, the anti-HIV gp120 V3 glycan and anti-HIV gp120 CD4bs binding antibodies described herein are combined with an interleukin receptor agonist, such as IL-2, IL-7, IL-15, IL-10, IL-12 agonists; examples of IL-2 receptor agonists such as proleukin (aldesleukin, IL-2); pegylated IL-2 (e.g., NKTR-214); modified variants of IL-2 (e.g., THOR-707), bempegaldesleukin, AIC-284, ALKS-4230, CUI-101, Neo-2/15; IL-15 receptor agonists, such as ALT-803, NKTR-255, and hetIL-15, interleukin-15/Fc fusion protein, AM-0015, NIZ-985, SO-C101, IL-15 Synthorin (pegylated IL-15), P-22339, and a IL-15-PD-1 fusion protein N-809; examples of IL-7 include CYT-107.

Examples of interferon receptor agonists that can be combined with the anti-HIV gp120 V3 glycan and anti-HIV gp120 CD4bs binding antibodies described herein include interferon alfa; interferon alfa-2b; interferon alfa-n3; pegylated interferon alfa; interferon gamma; gepon; normferon, peginterferon alfa-2a, peginterferon alfa-2b, RPI-MN.

In certain embodiments, the anti-HIV gp120 V3 glycan and anti-HIV gp120 CD4bs binding antibodies described herein are combined with a Flt3 agonist, such as GS-3583 or CDX-301.

### Bi- and Tri-Specific Natural Killer (NK)-Cell Engagers

In various embodiments, the anti-HIV gp120 V3 glycan and anti-HIV gp120 CD4bs binding antibodies described herein are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcTR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (4-1BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices, et al., Methods Mol Biol. (2016) 1441:333-346; Fang, et al., Semin Immunol. (2017) 31:37-54. Examples of a trispecific NK cell engager (TRiKE) include OXS-3550, HIV-TriKE and CD16-IL-15-B7H3 TriKe.

### Indoleamine-pyrrole-2,3-dioxygenase (IDO1) Inhibitors

In certain embodiments, the anti-HIV gp120 V3 glycan and anti-HIV gp120 CD4bs binding antibodies described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

### Phosphatidylinositol 3-Kinase (PI3K) Inhibitors

In certain embodiments, the anti-HIV gp120 V3 glycan and anti-HIV gp120 CD4bs binding antibodies described herein are combined with a PI3K inhibitor. Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

In certain embodiments, the anti-HIV gp120 V3 glycan and anti-HIV gp120 CD4bs binding antibodies described herein are combined with an alpha-4/beta-7 antagonist. Examples of Integrin alpha-4/beta-7 antagonists include PTG-100, TRK-170, abrilumab, etrolizumab, carotegrast methyl, and vedolizumab.

### HPK1/MAP4K1 Inhibitors

In certain embodiments, the anti-HIV gp120 V3 glycan and anti-HIV gp120 CD4bs binding antibodies described herein are combined with an inhibitor of mitogen-activated protein kinase kinase kinase kinase 1 (MAP4K1, a.k.a., Hematopoietic Progenitor Kinase 1 (HPK1); NCBI Gene ID: 11184). Examples of HPK1 inhibitors include, but are not limited to, ZYF-0272, and ZYF-0057.

### Pharmacokinetic Enhancers

In certain embodiments, the anti-HIV gp120 V3 glycan and anti-HIV gp120 CD4bs binding antibodies described herein are combined with a pharmacokinetic enhancer. Examples of pharmacokinetic enhancers include cobicistat and ritonavir.

### Additional Therapeutic Agents

Examples of additional therapeutic agents include the compounds disclosed in WO 2004/096286 (Gilead Sciences); WO 2006/015261 (Gilead Sciences); WO 2006/110157 (Gilead Sciences); WO 2012/003497 (Gilead Sciences); WO 2012/003498 (Gilead Sciences); WO 2012/145728 (Gilead Sciences); WO 2013/006738 (Gilead Sciences); WO 2013/159064 (Gilead Sciences); WO 2014/100323 (Gilead Sciences), US 2013/0165489 (University of Pennsylvania), US 2014/0221378 (Japan Tobacco), US 2014/0221380 (Japan Tobacco); WO 2009/062285 (Boehringer Ingelheim); WO 2010/130034 (Boehringer Ingelheim); WO 2013/006792 (Pharma Resources), US 20140221356 (Gilead Sciences), US 20100143301 (Gilead Sciences) and WO 2013/091096 (Boehringer Ingelheim).

### HIV Combination Therapy

In a particular embodiment, the anti-HIV gp120 V3 glycan and anti-HIV gp120 CD4bs binding antibodies described herein are combined with one, two, three, four or more additional therapeutic agents selected from ATRIPLA® (efavirenz, tenofovir disoproxil fumarate, and emtricitabine); BIKTARVY® (bictegravir+emtricitabine+tenofovir alafenamide), COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

It will be appreciated by one of skill in the art that the additional therapeutic agents listed above may be included in more than one of the classes listed above. The particular classes are not intended to limit the functionality of those compounds listed in those classes.

In a specific embodiment, the anti-HIV gp120 V3 glycan and anti-HIV gp120 CD4bs binding antibodies described herein are combined with an HIV nucleoside or nucleotide inhibitor of reverse transcriptase and an HIV non-nucleoside inhibitor of reverse transcriptase. In another specific embodiment, the anti-HIV gp120 V3 glycan and anti-HIV gp120 CD4bs binding antibodies described herein are combined with an HIV nucleoside or nucleotide inhibitor of reverse transcriptase, and an HIV protease inhibiting compound. In an additional embodiment, the anti-HIV gp120 V3 glycan and anti-HIV gp120 CD4bs binding antibodies described herein are combined with an HIV nucleoside or nucleotide inhibitor of reverse transcriptase, an HIV non-nucleoside inhibitor of reverse transcriptase, and a pharmacokinetic enhancer. In certain embodiments, the anti-HIV gp120 V3 glycan and anti-HIV gp120 CD4bs binding antibodies described herein are combined with at least one HIV nucleoside inhibitor of reverse transcriptase, an integrase inhibitor, and a pharmacokinetic enhancer. In another embodiment, the anti-HIV gp120 V3 glycan and anti-HIV gp120 CD4bs binding antibodies described herein are combined with two HIV nucleoside or nucleotide inhibitors of reverse transcriptase.

In a particular embodiment, the anti-HIV gp120 V3 glycan and anti-HIV gp120 CD4bs binding antibodies described herein are combined with abacavir sulfate, tenofovir, tenofovir disoproxil, tenofovir disoproxil fumarate, tenofovir disoproxil hemifumarate, tenofovir alafenamide, or tenofovir alafenamide hemifumarate.

In a particular embodiment, the anti-HIV gp120 V3 glycan and anti-HIV gp120 CD4bs binding antibodies described herein are combined with tenofovir, tenofovir disoproxil, tenofovir disoproxil fumarate, tenofovir alafenamide, or tenofovir alafenamide hemifumarate.

In a particular embodiment, the anti-HIV gp120 V3 glycan and anti-HIV gp120 CD4bs binding antibodies described herein are combined with a first additional therapeutic agent selected from the group consisting of abacavir sulfate, tenofovir, tenofovir disoproxil, tenofovir disoproxil fumarate, tenofovir alafenamide, and tenofovir alafenamide hemifumarate, and a second additional therapeutic agent selected from the group consisting of emtricitabine and lamivudine.

In a particular embodiment, the anti-HIV gp120 V3 glycan and anti-HIV gp120 CD4bs binding antibodies described herein are combined with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

In certain embodiments, the anti-HIV gp120 V3 glycan and anti-HIV gp120 CD4bs binding antibodies described herein are combined with one or more additional therapeutic agents in a therapeutically effective dosage amount in the range of e.g., from 1 mg to 50 mg, 75 mg, 100 mg, 150 mg, 200 mg, 250 mg, 300 mg, 400 mg, 500 mg, 1000 mg or 1500 mg of the anti-HIV gp120 V3 glycan and anti-HIV gp120 CD4bs binding antibodies or antigen-binding fragment. In certain embodiments, the anti-HIV gp120 V3 glycan and anti-HIV gp120 CD4bs binding antibodies described herein are combined with one or more additional therapeutic agents in a therapeutically effective dosage amount in the range of e.g., from about 0.1 mg/kg to about 0.5 mg/kg, 1 mg/kg, 2 mg/kg, 3 mg/kg, 4 mg/kg, 5 mg/kg, 8 mg/kg, 10 mg/kg, 15 mg/kg, 20 mg/kg, 25 mg/kg, 30 mg/kg, 35 mg/kg, 40 mg/kg, 45 mg/kg or 50 mg/kg of the anti-HIV gp120 V3 glycan and anti-HIV gp120 CD4bs binding antibodies or antigen-binding fragment. In certain embodiments, the anti-HIV gp120 V3 glycan and anti-HIV gp120 CD4bs binding antibodies described herein are combined with one or more additional therapeutic agents in a therapeutically effective dosage amount in the range of e.g., from about 5 mg to about 10 mg, 20 mg, 25 mg, 50 mg, 100 mg, 125 mg, 150 mg, 250 mg, 300 mg, 500 mg, 1000 mg or 1500 mg of the anti-HIV gp120 V3 glycan and anti-HIV gp120 CD4bs binding antibodies or antigen-binding fragment.

In certain embodiments, the anti-HIV gp120 V3 glycan and anti-HIV gp120 CD4bs binding antibodies described herein are combined with 5-30 mg tenofovir alafenamide fumarate, tenofovir alafenamide hemifumarate, or tenofovir alafenamide, and 200 mg emtricitabine. In certain embodiments, the anti-HIV gp120 V3 glycan and anti-HIV gp120 CD4bs binding antibodies described herein are combined with 5-10, 5-15, 5-20, 5-25, 25-30, 20-30, 15-30, or 10-30 mg tenofovir alafenamide fumarate, tenofovir alafenamide hemifumarate, or tenofovir alafenamide, and 200 mg emtricitabine. In certain embodiments, the anti-HIV gp120 V3 glycan and anti-HIV gp120 CD4bs binding antibodies described herein are combined with 10 mg tenofovir alafenamide fumarate, tenofovir alafenamide hemifumarate, or tenofovir alafenamide, and 200 mg emtricitabine. In certain embodiments, the anti-HIV gp120 V3 glycan and anti-HIV gp120 CD4bs binding antibodies described herein are combined with 25 mg tenofovir alafenamide fumarate, tenofovir alafenamide hemifumarate, or tenofovir alafenamide, and 200 mg emtricitabine.

In certain embodiments, the anti-HIV gp120 V3 glycan and anti-HIV gp120 CD4bs binding antibodies described herein are combined with 200-400 mg tenofovir disoproxil fumarate, tenofovir disoproxil hemifumarate, or tenofovir disoproxil, and 200 mg emtricitabine. In certain embodiments, the anti-HIV gp120 V3 glycan and anti-HIV gp120 CD4bs binding antibodies described herein are combined with 200-250, 200-300, 200-350, 250-350, 250-400, 350-400, 300-400, or 250-400 mg tenofovir disoproxil fumarate, tenofovir disoproxil hemifumarate, or tenofovir disoproxil, and 200 mg emtricitabine. In certain embodiments, the anti-HIV gp120 V3 glycan and anti-HIV gp120 CD4bs binding antibodies described herein are combined with 300 mg tenofovir disoproxil fumarate, tenofovir disoproxil hemifumarate, or tenofovir disoproxil, and 200 mg emtricitabine. The anti-HIV gp120 V3 glycan and anti-HIV gp120 CD4bs binding antibodies may be combined with the agents provided herein in any dosage amount (e.g., from 1 mg to 500 mg of the anti-HIV gp120 V3 glycan and anti-HIV gp120 CD4bs binding antibodies) the same as if each combination of dosages were specifically and individually listed.

### Long-Acting HIV Inhibitors

In some embodiments, the anti-HIV gp120 V3 glycan and anti-HIV gp120 CD4bs binding antibodies described herein can be co-administered with a long-acting HIV inhibitor. In various embodiments, the long-acting HIV inhibits can be co-administered twice annually, e.g., every 6 months (Q6M), every 24 weeks (Q24W), every 25 weeks (Q25W), every 26 weeks (Q26W). Examples of long-acting HIV inhibitors that can be combined or co-administered include without limitation: long-acting capsid inhibitors, e.g., lenacapavir; long-acting integrase inhibitors, e.g., long acting bictegravir (GS-9883), GS-6212, cabotegravir long-acting (LA), long-acting raltegravir (RAL); long-acting NRTIs, e.g., EFdA/MK-8591 (4-ethynyl-2-fluoro-2-deoxyadenosine; islatravir) implant, tenofovir alafenamide fumarate (TAF) implant, injectable rovafovir etalafenamide (GS-9131); long-acting NNRTIs, e.g., GS-5894, long-acting dapivirine (DPV), long-acting rilpivirine (RPV), Elsulfavirine; also, VM-1500 LAI, maraviroc (LAI), and long-acting dolutegravir, (RPV). Long-acting anti-HIV drugs are reviewed in Singh, et al., Pharmaceuticals (2019) 12:62.

### HIV Vaccines

In certain embodiments, the anti-HIV gp120 V3 glycan and anti-HIV gp120 CD4bs binding antibodies described herein are combined with an HIV vaccine. Examples of HIV vaccines include peptide vaccines, recombinant subunit protein vaccines, live vector vaccines, DNA vaccines, HIV MAG DNA vaccines, CD4-derived peptide vaccines, vaccine combinations, adenoviral vector vaccines (e.g., Ad5, Ad26 or Ad35), simian adenovirus (chimpanzee, gorilla, rhesus i.e., rhAd), adeno-associated virus vector vaccines, chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, bi-segmented or tri-segmented arenavirus based vaccines (e.g., LCMV, Pichinde), trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as Vesicular stomatitis virus (VSV) and marabavirus; recombinant human CMV (rhCMV) based vaccine, alphavirus-based vaccines, such as semliki forest virus, venezuelan equine encephalitis virus and sindbis virus (see, e.g., Lauer, et al., Clin Vaccine Immunol. (2017) 24(1): e00298-16); LNP formulated mRNA based therapeutic vaccines; and LNP-formulated self-replicating RNA/self-amplifying RNA vaccines.

Examples of HIV vaccines include without limitation AAVLP-HIV vaccine, AdC6-HIVgp140, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, rgp120 (AIDSVAX), ALVAC HIV (vCP1521)/AIDSVAX B/E (gp120) (RV144), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, exoVACC, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, recombinant peptide vaccine (HIV infection), NCI, rgp160 HIV vaccine, RNActive HIV vaccine, SCB-703, Tat Oyi vaccine, TBC-M4, therapeutic HIV vaccine, UBI HIV gp120, Vacc-4x+romidepsin, variant gp120 polypeptide vaccine, rAd5 gag-pol env A/B/C vaccine, DNA.HTI and MVA.HTI, MVA.tHIVconsv3, MVA.tHIVconsv4, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based vaccines, VPI-211, HIV ANTI-CD40.ENV GP140, HIV ANTI-CD40.HIV5PEP, multimeric HIV gp120 vaccine TBL-1203HI, CH505 TF chTrimer, CD40.HIVRI.Env vaccine, VRC-HIVRGP096-00-VP, Drep-HIV-PT-1, BG505 MD39.3 mRNA, BG505 MD39.3 gp151 CD4KO mRNA, BG505 MD39.3 gp151 mRNA, mRNA-1644, mRNA-1547, mRNA-1574 and anti-HIV vaccines described in WO2021011544 and WO2022155258.

### Birth Control (Contraceptive) Combination Therapy

In certain embodiments, the anti-HIV gp120 V3 glycan and anti-HIV gp120 CD4bs binding antibodies described herein are combined with a birth control or contraceptive regimen. Therapeutic agents used for birth control (contraceptive) include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the anti-HIV gp120 V3 glycan and anti-HIV gp120 CD4bs binding antibodies described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include LB-1903, ENOB-HV-01, ENOB-HV-21, ENOB-HV-31, GOVX-B01, HSPCs overexpressing ALDH1 (LV-800, HIV infection), AGT103-T, and SupT1 cell-based therapy. Examples of dendritic cell therapy include AGS-004. CCR5 gene editing agents include without limitation SB-728T and SB-728-HSPC. CCR5 gene inhibitors include Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the anti-HIV gp120 V3 glycan and anti-HIV gp120 CD4bs binding antibodies described herein are co-administered with AGT-103-transduced autologous T-cell therapy or AAV-eCD4-Ig gene therapy.

### Gene Editors

In certain embodiments, the anti-HIV gp120 V3 glycan and anti-HIV gp120 CD4bs binding antibodies described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101 and XVIR-TAT.

### CAR-T-Cell Therapy

In some embodiments, the anti-HIV gp120 V3 glycan and anti-HIV gp120 CD4bs binding antibodies described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N-glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-Env duoCAR T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR-T-Cell Therapy

In certain embodiments, the anti-HIV gp120 V3 glycan and anti-HIV gp120 CD4bs binding antibodies described herein are combined with a population of TCR-T-cells. TCR-T-cells are engineered to target HIV derived peptides present on the surface of virus-infected cells, for example, IMC-M113V, a TCR bispecific having a TCR binding domain that targets a peptide derived from the Gag protein presented by HLA*A02 on the surface of HIV infected cells and a second antigen binding domain that targets CD3.

6. Kits

Further provided are kits comprising one or more unitary doses of a first antibody that binds HIV gp120 V3 glycan and a second antibody that binds HIV gp120 CD4bs, wherein the first antibody and the second antibody have serum half-life extending amino acid substitutions, and the first antibody and the second antibody are formulated for administration twice annually (e.g., every 6 months (Q6M), every 26 weeks (Q26W), every 25 weeks (Q25W), or every 24 weeks (Q24W)).

In certain embodiments, the kit comprises the anti-HIV gp120 V3 glycan binding antibody and the anti-HIV gp120 CD4bs binding antibody, as described herein, are combined in a unitary dosage form, separately or as a mixture, for simultaneous administration to a patient, for example as a liquid or suspension dosage form for intravenous, intramuscular or subcutaneous administration.

In some embodiments, the unitary doses of a first antibody that binds HIV gp120 V3 glycan and a second antibody that binds HIV gp120 CD4bs independently are in the range of from about 500 mg to about 3000 mg, e.g., from about 550 mg to about 2900 mg, e.g., from about 600 mg to about 2800 mg, e.g., from about 650 mg to about 2700 mg, e.g., from about 700 mg to about 2600 mg, e.g., from about 850 mg to about 2550 mg. In some embodiments, the unitary dose of the anti-HIV gp120 V3 glycan binding antibody (e.g., 10-1074-LS) is 850 mg. In some embodiments, the unitary dose of the anti-HIV gp120 V3 glycan binding antibody (e.g., 10-1074-LS) is 2550 mg. In some embodiments, the unitary dose of the anti-HIV gp120 CD4bs binding antibody (e.g., 3BNC117-LS) is 2550 mg. In some embodiments, the unitary doses of the anti-HIV gp120 V3 glycan and anti-HIV gp120 CD4bs binding antibodies (e.g., 3BNC117-LS) are both 1700 mg. In some embodiments, the unitary dose of the anti-HIV gp120 V3 glycan and anti-HIV gp120 CD4bs binding antibodies (e.g., 3BNC117-LS) are both 2550 mg. In some embodiments, the unitary dose of the anti-HIV gp120 V3 glycan binding antibody (e.g., 10-1074-LS) is 850 mg and the unitary dose of the anti-HIV gp120 CD4bs binding antibody (e.g., 3BNC117-LS) is 2550 mg. In some embodiments, the unitary dose of the anti-HIV gp120 V3 glycan binding antibody (e.g., 10-1074-LS) is 850 mg and the unitary dose of the anti-HIV gp120 CD4bs binding antibody (e.g., 3BNC117-LS) is 1700 mg. In some embodiments, the unitary dose of the anti-HIV gp120 V3 glycan binding antibody (e.g., 10-1074-LS) is 850 mg and the unitary dose of the anti-HIV gp120 CD4bs binding antibody (e.g., 3BNC117-LS) is 1275 mg.

In some embodiments, the kit further comprises one or more unitary does of a long-acting anti-HIV drug. In some embodiments, the one or more long-acting HIV drugs are selected from a long-acting capsid inhibitor, a long-acting integrase strand transfer inhibitor (INSTI), a long-acting non-nucleoside reverse transcriptase inhibitor (NNRTI), a long-acting nucleoside reverse transcriptase inhibitors (NRTI), and a long-acting protease inhibitor (PI). In some embodiment, the long-acting capsid inhibitor comprises lenacapavir. In some embodiments, the unitary dose of lenacapavir is in the range of from 300 mg to 1000 mg, e.g., 300 mg, 600 mg, 900 mg, 927 mg. As appropriate, the unitary doses of lenacapavir can be formulated for oral, subcutaneous or intravenous administration. In some embodiments, the long-acting INSTI is selected from bictegravir, raltegravir, elvitegravir, dolutegravir, and cabotegravir. In some embodiments, the long-acting NNRTI is selected from rilpivirine, elsulfavirine, doravirine and GS-5894. In some embodiments, the long-acting NRTI is selected from islatravir and prodrugs thereof, tenofovir alafenamide (TAF) and prodrugs of tenofovir, rovafovir etalafenamide and GS-1614. In some embodiments, the long-acting protease inhibitor is selected from atazanavir, ritonavir, darunavir, GS-1156 and prodrugs of GS-1156, and combinations thereof.

In one embodiment, the kit comprises one or more pharmaceutical packs or one or more containers (e.g., vials, ampules, preloaded syringes) containing one or more of the ingredients of the pharmaceutical compositions described herein, such an anti-HIV gp120 V3 glycan binding antibody and an anti-HIV gp120 CD4bs binding antibody described herein. In some instances, the kits contain a pharmaceutical composition described herein. In one embodiment, kits comprising an anti-HIV gp120 V3 glycan binding antibody and an anti-HIV gp120 CD4bs binding antibody described herein, or a pharmaceutical composition thereof, in combination with one or more (e.g., one, two, three, four, one or two, one to three, or one to four) additional therapeutic agents (such as those disclosed above) are provided.

Optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLES

The following examples are offered to illustrate, but not to limit the claimed invention.

### Example 1

### Ph1b Study: 26 W Primary Outcomes of Long Acting Broadly Neutralizing Antibodies in Combination with Lenacapavir

GS-US-536-5816 (NCT04811040 on ClinicalTrials.gov) is a randomized, blinded, proof-of-concept (POC) Phase 1b study to evaluate the safety and efficacy of a single dose each of a long acting regimen of lenacapvir, teropavimab (GS 5423; 3BNC117-LS; TAB) and zinlirvimab (GS 2872; 10-1074-LS; ZAB) in adults with HIV-1 infection who are virologically suppressed (HIV-1 RNA <50 copies/mL) on oral ART.

### Dosing

Participants were adults living with HIV virologically-suppressed ≥2 years (HIV-1 RNA <50 copies/mL) on ART, sensitive to both bNAbs by HIV proviral DNA phenotype (PhenoSense mAb IC90 ≤2 ug/mL, Monogram Biosciences), a CD4 nadir ≥350, and CD4 count ≥500 at study entry. Participants who provided written consent and met all eligibility criteria were randomized in a 1:1 ratio to 1 of 2 treatment groups based on the dose of GS 2872 (10 mg/kg or 30 mg/kg administered IV). All participants received GS-5423 (30 mg/kg IV) and oral lenacapavir 600 mg Day 1 and Day 2 and lenacapavir for injection 927 mg subcutaneously on Day 1. Participants were monitored clinically with plasma HIV-1 RNA every four weeks until the primary endpoint at Week 26. The primary endpoint was safety; secondary endpoints included virologic outcomes by FDA Snapshot analysis.

In a first-in-human study of 3BNC117-LS (NCT03254277), 39 received a single dose of 3BNC117-LS at doses ranging from 3 to 30 mg/kg (IV) or 150 or 300 mg (SC); 5 participants received placebo. Five of 43 enrolled participants reported 5 solicited adverse events (AEs) within 4 weeks following dosing, all of Grade 1 severity: tenderness at administration site (2%), headache (2%), malaise/fatigue (2%), and nausea (4%). In addition, 48 nonsolicited AEs were reported by 28 of 43 enrolled participants, and 29 of the reported events (58%) occurred within 4 weeks of investigational product (IP) administration. Of the reported events, 9 were of Grade 2 severity (17%) and 2 were of Grade 3 severity (4%): proteinuria and cellulitis that required admission for IV antibiotics), 1 was of Grade 4 severity (hypokalemia). One participant was admitted with a transient ischemic attack secondary to a right carotid artery thrombus. Further evaluation revealed a vascular anatomical abnormality which likely led to the thrombotic event. This serious AE was considered not related to the IP. The most commonly reported AEs were those related to upper respiratory infections (14%), nausea (4%), and dizziness (4%).

In a first-in-human study of 10-1074-LS (NCT03554408), 77 participants enrolled: 27 participants received a single dose of 10-1074-LS at doses ranging from 3 to 30 mg/kg (IV, n=15) or 140 or 280 mg (SC, n=12); 12 additional participants received a single SC injection of the combination of 10-1074-LS and 3BNC117-LS, and 18 received 3 repeated SC injections (every 12 weeks) of the antibody admixture; 10 participants received a single intravenous infusion of 10-1074-LS and 3BNC117-LS at a dose of 30 mg/kg of each antibody. The remaining 10 participants received placebo. As of July 2020, 20 solicited AEs were reported by 15 out of 77 enrolled participants, all of Grade 1 severity: erythema/skin discoloration (8%), pain (4%), and induration (2%) at the administration site, headache (4%), feverishness (4%), malaise/fatigue (3%), and myalgia (1%). In addition, 86 non-solicited AEs were reported by 46 participants. Of these, 86 AEs, 29 (33.7%) occurred within 4 weeks of IP administration. Of the reported non-solicited AEs, 10 were of grade 2 severity (11.6%) and 8 reported events were of Grade 3 severity (9.3%): nephrolithiasis (1%), elevated blood pressure (4%), decrease in hemoglobin (1%), proteinuria (1%), and increased left-sided weakness (1%). The 3 participants who experienced transient Grade 3 elevation in blood pressure on the day of IP administration had preexisting history of hypertension. The most common AEs were those related to upper respiratory infections (25%), localized musculoskeletal pain (8%) and symptoms of gastroenteritis (8%).

### NCT04811040 Ph1b Study Summary

Participants discontinued their background oral ART regimen 1 day prior to receiving study drugs on Day 1. Of 124 screened participants, 55 were sensitive to both bNAbs, 21 were randomized, and 20 received the complete study regimen. The median age was 44 yrs (IQR 34, 51); 14% were female; 14% Black, 14% Asian, 33% Hispanic/Latinx; median CD4 count was 909 (IQR 687, 1270).

At Week 26, all participants resumed their background oral ART baseline regimen (or compatible regimen selected by the investigator) and returned to the clinic for visits at Weeks 38 and 52.

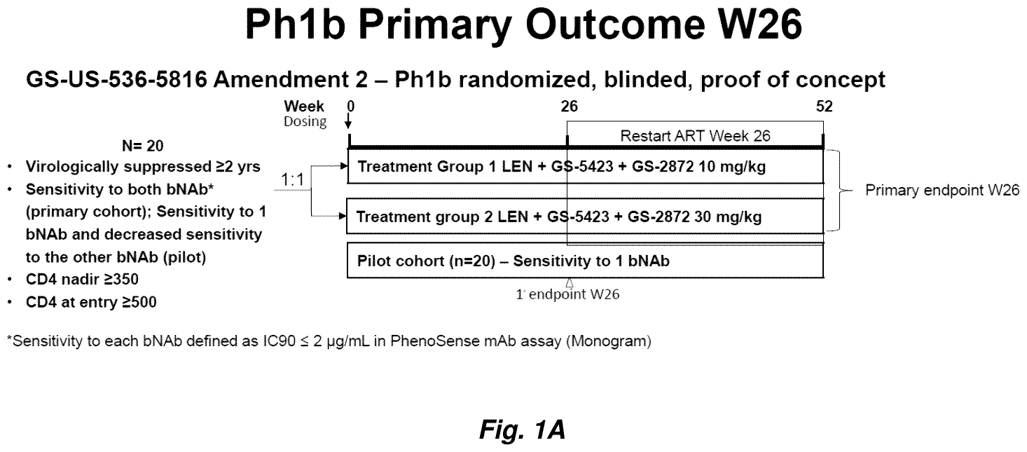

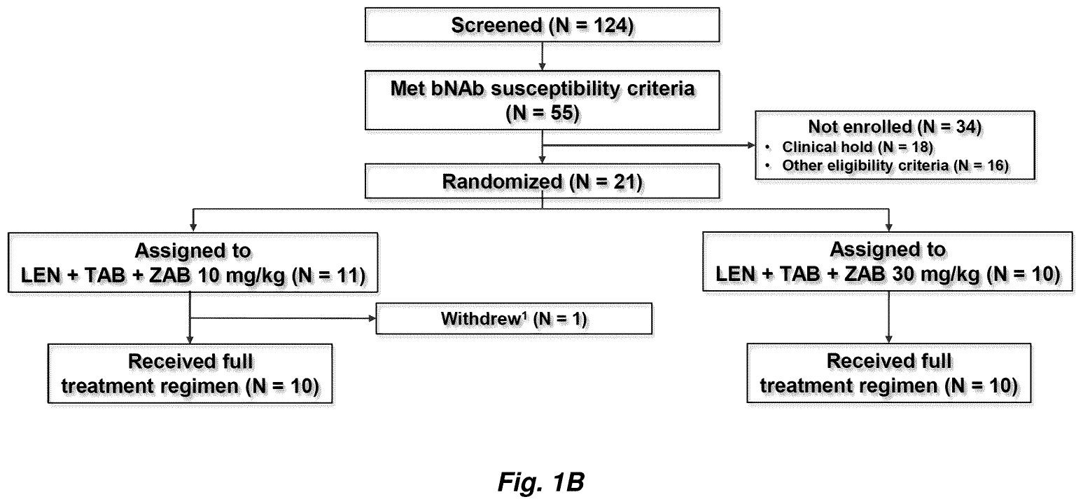

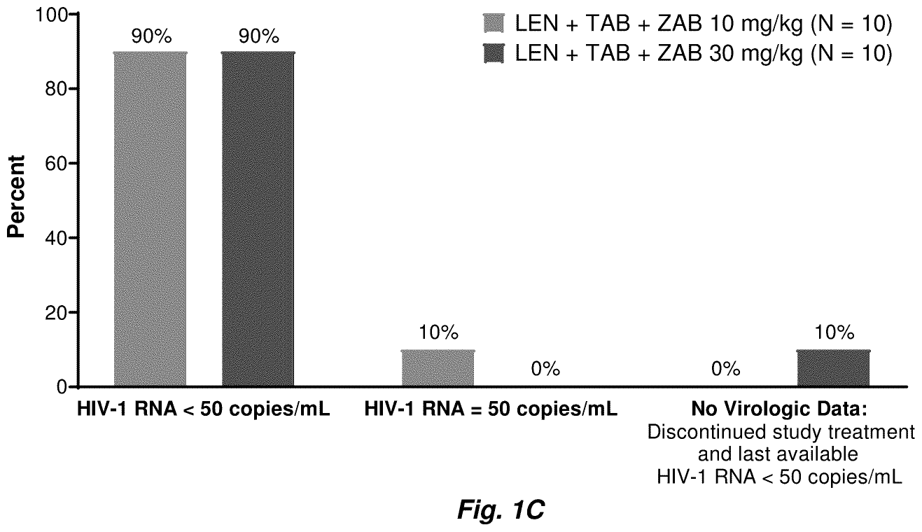

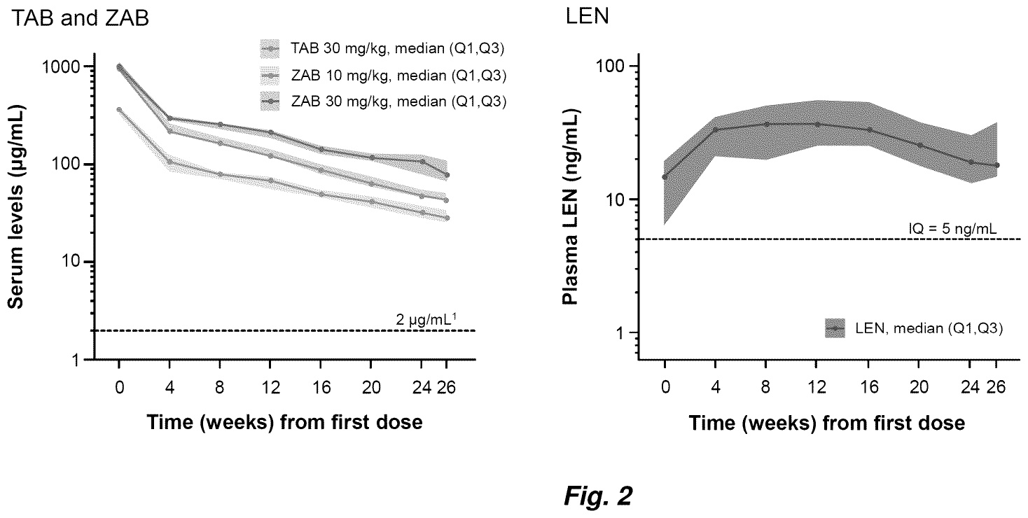

Approximately 20 participants were in the Primary Cohort. Adults with HIV-1, no history of virologic failure (VF) or antiretroviral drug resistance, a CD4 nadir ≥350 cells/μL, on first line ART for at least 2 years with demonstrated virologic suppression (HIV-1 RNA <50 copies/mL) for at least 18 months prior to screening who were willing to modify their ART regimen for an investigational strategy. A schematic of the study is provided in FIG. 1.

21 participants were enrolled into the primary cohort and randomized, 20 participants received the complete study regimen (10 in each treatment group), one participant received oral lenacapavir and withdrew consent prior to completing dosing procedures. The median age of participants was 44 years (range 25-61), 18 (86%) were male sex at birth, all had HIV-1 RNA <50 copies/mL and CD4 count >500 cells/μL. Enrolled participant demographics and baseline characteristics are summarized in Table 1.

Therapeutic concentrations of teropavimab (TAB), zinlirvimab (ZAB) and lenacapavir (LEN) were maintained through Week 26. These results are depicted in FIG. 1B.

TABLE 1
Enrolled Participant Demographics and Baseline Characteristics

| | LEN + TAB + ZAB | | LEN + TAB + ZAB | | Total | |
|---|---|---|---|---|---|---|
| | 10 mg/kg (N = 11) | | 30 mg/kg (N = 10) | | N = 21 | |
| Age, median (range) | 46 | (31 to 61) | 37 | (25 to 59) | 44 | (25 to 61) |

| | | | | |
|---|---|---|---|---|
| Sex at birth, n | Male | 11 | 7 | 18 |
| | Female | 0 | 3 | 3 |
| Race, n | Asian | 2 | 1 | 3 |
| | Black | 1 | 2 | 3 |
| | White | 7 | 5 | 12 |
| | Other | 1 | 2 | 3 |

| Hispanic or Latino ethnicity, n | 4 | | 3 | | 7 | |
|---|---|---|---|---|---|---|
| Weight (kg), median (range) | 90.2 | (58.9 to 150.0) | 92.9 | (60.2 to 143.0) | 90.2 | (58.9 to 150.0) |
| Body mass index (kg/m²), median (range) | 30.2 | (21.6 to 42.9) | 30.2 | (21.6 to 54.1) | 30.2 | (21.6 to 54.1) |
| CD4 cell count (per mL), median (range) | 778 | (547 to 1391) | 1024 | (667 to 1644) | 909 | (547 to 1644) |
| Duration of baseline ART (years), median (range) | 3.6 | (2.4 to 4.8) | 2.6 | (2.0 to 5.5) | 2.6 | (2.0 to 5.5) |
| Time since HIV diagnosis (years), median (range) | 12.4 | (6.4 to 26.3) | 5.3 | (2.6 to 22.4) | 8.2 | (2.6 to 26.3) |

Efficacy was assessed at the week 26 primary endpoint according to the FDA Snapshot algorithm. One participant in Group 1 had a confirmed HIV RNA ≥50 copies/mL (155 copies/mL, confirmed 524 copies/mL) at Week 16 and resuppressed with re-initiation of baseline ART; one participant in Group 2 withdrew consent at Week 12 (with HIV-1 RNA <50 copies/mL). 18/20 (90%) participants had HIV-1 RNA <50 copies/mL at Week 26. Primary efficacy results are summarized in Table 2 and FIG. 1C.

TABLE 2
Efficacy as Determined by the US FDA-defined Snapshot Algorithm at Week 26

| | LEN + GS-5423 + | LEN + GS-5423 + |
|---|---|---|
| | GS-2872 10 mg/kg | GS-2872 30 mg/kg |
| | (N = 10) | (N = 10) |
| HIV-1 RNA ≥50 copies/mL, N | 1\hat{ } | 0 |
| (% [95% CI]) | (10%, [0.3%, 44.5%]) | |
| HIV-1 RNA <50 copies/mL, N | 9 | 9 |
| (% [95% CI]) | (90%, [55.5%, 99.7%]) | (90%, [55.5%, 99.7%]) |
| Discontinued Study Drug Due to | 0 | 1 (10%)* |
| Other Reasons^{$} and Last | | |
| Available HIV-1 RNA <50 | | |
| copies/mL | | |

\hat{ }Resistance tests pending
*Withdrew from the study after week 12
^{$}Reasons other than AE/Death or lack of efficacy

There were no treatment emergent serious adverse events, no treatment emergent adverse events leading to discontinuation of study drug or study and no deaths. The most common treatment emergent adverse events were injection site reactions related to administration of subcutaneous lenacapavir (LEN) (17/20 patients or 85%). Two participants had grade 3 AEs: one with injection site cellulitis and one with injection site erythema at the site of LEN injection. The combination of LEN+GS-5423 (teropavimab)+GS-2872 (zinlirvimab) was well-tolerated with high efficacy for 6 months in selected virologically-suppressed persons living with HIV. These results are consistent with the conclusion that the LEN+GS-5423 (teropavimab)+GS-2872 (zinlirvimab) combination provides long-acting treatment for HIV with twice-yearly dosing.

### Example 2

### Modeling to Determine Flat Dosing that Allows for Twice Annual Administration

In this example, we performed population PK (popPK) modeling and simulation to predict PK profiles of GS-5423 (teropavimab) and GS-2872 (zinlirvimab) with body-weight based dosing and flat-dosing at different doses and compared them with the target efficacious levels to determine the optimal dose range of GS-5423 and GS-2872 with every 6 month dosing in adults with HIV.

### Methods

PK data for GS-5423 (teropavimab; 3BNC117-LS; TAB) and GS-2872 (zinlirvimab; 10-1074-LS; ZAB) were obtained from four clinical studies in viremic or virally suppressed PWH (TAB: n=34; ZAB: n=36) who received single intravenous doses of TAB (30 mg/kg) and/or ZAB (10 or 30 mg/kg) alone or in combination with or without LEN, the studies including YCO-0946 (NCT03254277) and YCO-0971 (NCT03554408). TAB and ZAB serum concentrations were measured using validated Mesa Scale Discovery-electrochemiluminescence immunoassays. A two-compartment population PK model was developed to describe the PK data of GS-5423 and GS-2872 following IV and SC administration in HIV− and HIV+ participants. See, Joel S. Owen, Jill Fiedler-Kelly, “Introduction to Population Pharmacokinetic/Pharmacodynamic Analysis with Nonlinear Mixed Effects Models”, Wiley; 1st edition, 2014 (ISBN: 9780470582299). PopPK models of TAB and ZAB were developed using nonlinear mixed-effect modeling. Covariate analyses were performed to identify significant covariates, including body weight, effects of demographics, baseline characteristics, combination regimen, and disease status, on the PK parameters of GS-5423 and GS-2872. The population PK models were simulated to predict the PK profiles of GS-5423 and GS-2872 following IV administration of 30 or 10 mg/kg body weight normalized dosing or equivalent flat doses every 6 months. Model simulations were performed to predict the concentrations of TAB and ZAB following flat vs weight-based dosing. The distribution of body weight is assumed to be consistent with previous studies in adults with HIV virologically suppressed on anti-retroviral therapy (with mean body weight of 85 kg).

### Results

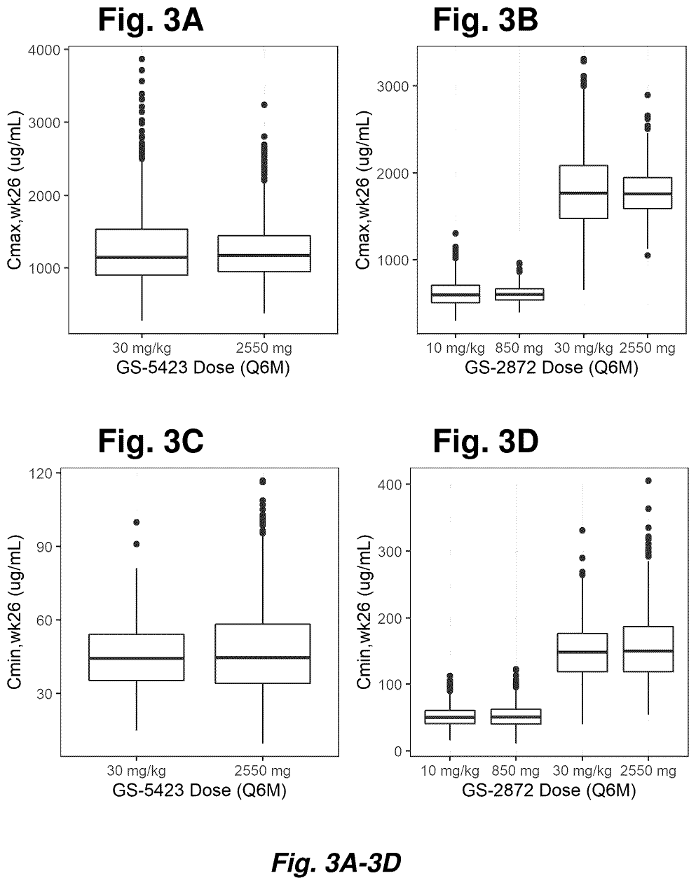

Simulations based on the PK modeling of the data from the four clinical studies, including YCO-0946 and YCO-0971, showed that, fixed doses of 2550 mg or 850 mg of GS-5423 or GS-2872 are expected to produce similar exposures as 30 mg/kg or 10 mg/kg weight-based dose, respectively, with no meaningful increase in PK variability (FIGS. 3A-3D). Therefore, doses up to 2550 mg IV is expected to be safe for both GS-5423 and GS-2872 in adults with HIV infection, given that GS-5423 and GS-2872 up to 30 mg/kg alone or in combination were well-tolerated in the on-going study GS-US-536-5816 and previous studies in HIV+ participants.

GS-5423 (TAB) and GS-2872 (ZAB) PK data in PWH were adequately described by two-compartment PopPK models. Increased body weight was associated with increased volume of distribution and clearance of both TAB and ZAB. PWH who were viremic had a significant increase in the clearance of TAB and ZAB compared with those who were suppressed at baseline. Model simulations suggest that a flat dose of 2550 mg would result in similar exposures as 30 mg/kg for both TAB and ZAB, based on the body weight distribution in recent Phase 3 HIV studies of adult PWH, with an average body weight of about 85 kg.

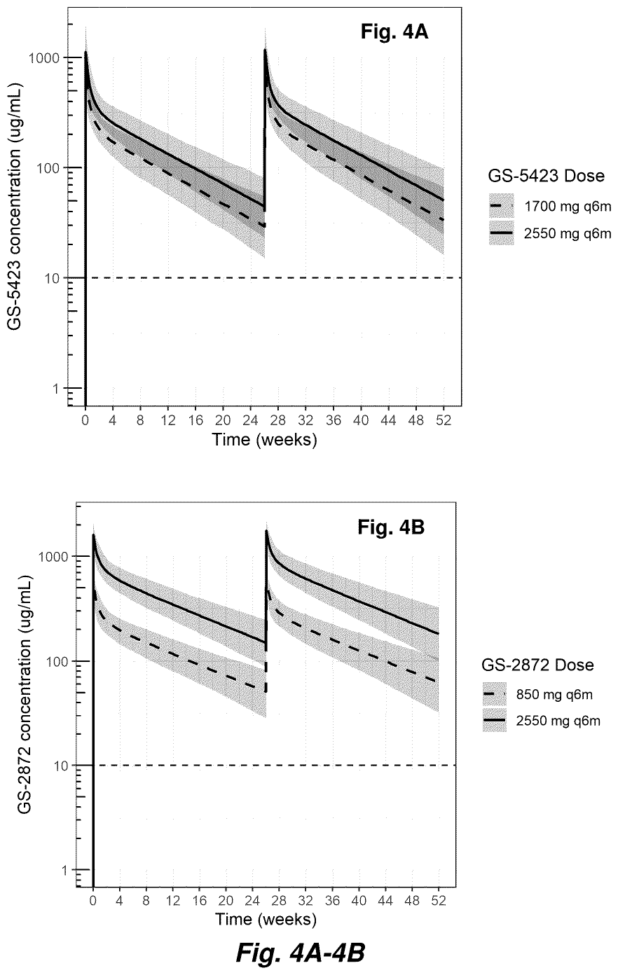

Previous studies of the non-LS forms of each antibody in HIV+ participants undergoing analytical treatment interruption (Mendoza, et al., Nature. (2018) 561(7724):479-484 and Gaebler, et al., Nature (2022) 606(7913):368-374) have shown that the virological suppression was generally maintained when serum concentrations of both antibodies were above 10 μg/mL. Based on the PK simulations, 1700 mg GS-5423 or 850 mg GS-2872 is anticipated to maintain the concentration above 10 μg/mL in 99%-100% of subjects through 6 months (26 weeks) after dosing (FIGS. 4A-4B, Table 3). Therefore, the dose range of 1700 to 2550 mg GS-5423 and 850 to 2550 mg GS-2872 given IV every 6 months are expected to be the efficacious and safe dose ranges for the two bNAbs.

TABLE 3
Predicted percentage of patients above 10 μg/mL at Week 26
after IV administration of GS-5423 and GS-2872 every 6 months

| | GS-5423 | | GS-2872 | |
|---|---|---|---|---|
| | 2550 mg | 1700 mg | 2550 mg | 850 mg |
| % Patients above | 100% | 99% | 100% | 100% |
| 10 μg/mL at Week 26 | | | | |

### Example 3

### Evaluation of Therapeutic Concentrations of Anti-HIV Antibodies 3BNC117/Teropavimab and 10-1074/Zinlirvimab Through PK-PD Modeling and Prediction of the Washout Duration in HIV Cure Studies

3BNC117 and 10-1074 have been shown to induce rapid decline in viremia in people with HIV, as well as delay the time to viral rebound in suppressed people with HIV during analytical treatment interruption (ATI) (Caskey, et al. Nature. 2015; 522:487-491; Caskey, et al. Nat Med. 2017; 23:185-191; Scheid, et al. Nature. 2016; 535:556-560; Mendoza, et al. Nature. 2018; 561:479-484; Bar-On, et al. Nat Med. 2018; 24:1701-1707; Gaebler, et al. Nature. 2022; 606:368-374). The combination of 3BNC117/TAB and 10-1074/ZAB, together with immune-modulating agents, is being investigated for its potential to eliminate the HIV reservoir and induce long-term remission in people with HIV. However, due to their potent viral neutralization effects, insufficient washout duration before ATI can confound the efficacy assessment of time to virologic rebound in HIV cure studies. The purpose of this study was to characterize the pharmacokinetics (PK) and pharmacokinetic-pharmacodynamic (PK-PD) relationships of these bNAbs through PK-PD viral dynamic modeling, and to predict the required length of washout for TAB/ZAB in HIV cure studies in order to assess post-treatment viral control during ATI.

### Methods

Population PK and PK-PD models were developed using a nonlinear mixed-effect modeling approach based on serum bNAb concentration and/or viral dynamic data from 6 efficacy studies in people with HIV, and 3 PK studies of 3BNC117/TAB (GS-5423) and/or 10-1074/ZAB (GS-2872) (Table 4).

TABLE 4
Studies Included in the PK-PD Modeling

| Study | Compound (dose) | Participants | Efficacy evaluation | N for PK | N for PD |
|---|---|---|---|---|---|
| NCT02018510 | 3BNC117 (1, 3, 10, 30 mg/kg IV) | HIV negative | | 22 | — |
| | | Suppressed PWH | | 16 | — |
| | | Viremic PWH | Viral suppression | 17 | 17 |
| NCT02511990 | 10-1074 (3, 10, 30 mg/kg IV) | HIV negative | | 14 | — |
| | | Suppressed PWH | | 3 | — |
| | | Viremic PWH | Viral suppression | 15 | 15 |
| NCT02446847 | 3BNC117 (30 mg/kg IV) | Suppressed PWH | Viral rebound during ATI | 15 | 14 |
| NCT02824536 | 3BNC117 + 10-1074 (3 + 3, 10 + 10 mg/kg IV) | HIV negative | — | 18 | — |
| NCT02825797 | 3BNC117 + 10-1074 (30 + 30 mg/kg IV) | Viremic PWH | Viral suppression | 7 | — |
| | | Suppressed PWH | Viral rebound during ATI | 21 | 13 |
| NCT03526848 | 3BNC117 + 10-1074 (30 + 30 mg/kg IV) | Suppressed PWH | Viral rebound during ATI | 26 | 22 |
| NCT03254277 | TAB (30 mg/kg IV, 150 or 300 mg SC) | HIV negative | — | 15 | — |
| | | Suppressed PWH | — | 3 | — |
| NCT03554408 | ZAB alone (3, 10, 30 mg/kg IV, 140 or 280 SC) | HIV negative | — | 57 | — |
| | TAB + ZAB (30 + 30 mg/kg IV, 150-300 + 60- | Suppressed PWH | — | 10 | — |
| | 280 mg SC) | | | | |
| NCT04250636 | TAB + ZAB (30 + 30 mg/kg IV) | Viremic PWH | Viral suppression | 6 | 6 |

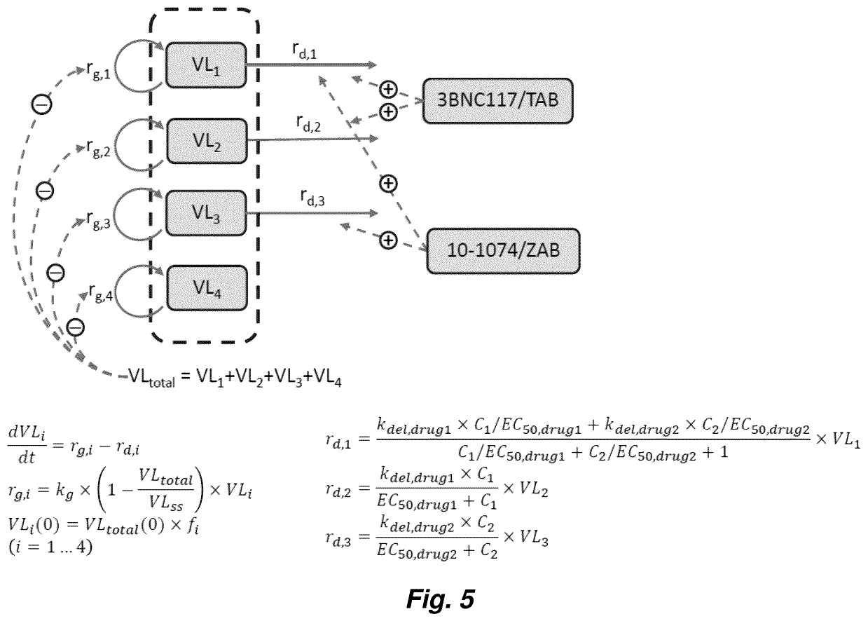

bNAb concentrations were measured by ELISA assays, except for study NCT03526848 (Gaebler, et al. Nature. 2022; 606:368-374). For this study, concentrations measured by TZM-bl assay (Sarzotti-Kelsoe, et al. J Immunol Methods. 2014; 409:131-146) were transformed to ELISA data using a log-linear correlation model calibrated based on data from study NCT02825797 (Mendoza, et al. Nature. 2018; 561:479-484; Bar-On Y, et al. Nat Med. 2018; 24:1701-1707) where PK was measured using both methods. The PK data of the bNAbs were modeled by 2-compartment linear PK models. Covariates (demographics, disease status, combination treatment) were tested using stepwise forward addition (α=0.01) and backward elimination (α=0.001) methods. The PK-PD model describes viral replication using a logistic growth function and viral elimination using first-order kinetics, with a nonlinear saturable (Emax) model to describe the relationship between bNAb concentrations and viral elimination rates. Distinct viral populations sensitive or resistant to each bNAb were modeled to capture the mechanism of resistance selection in treated participants (FIG. 5). PK and PK-PD models were fitted sequentially. Model evaluations were performed using standard diagnostic plots and visual predictive checks. Simulations were performed to predict PK and dynamics of viral rebound during ATI after different lengths of washout periods after TAB/ZAB dosing. Modeling was conducted using Phoenix® NLME. Simulations and plotting were performed using R software.

### Results

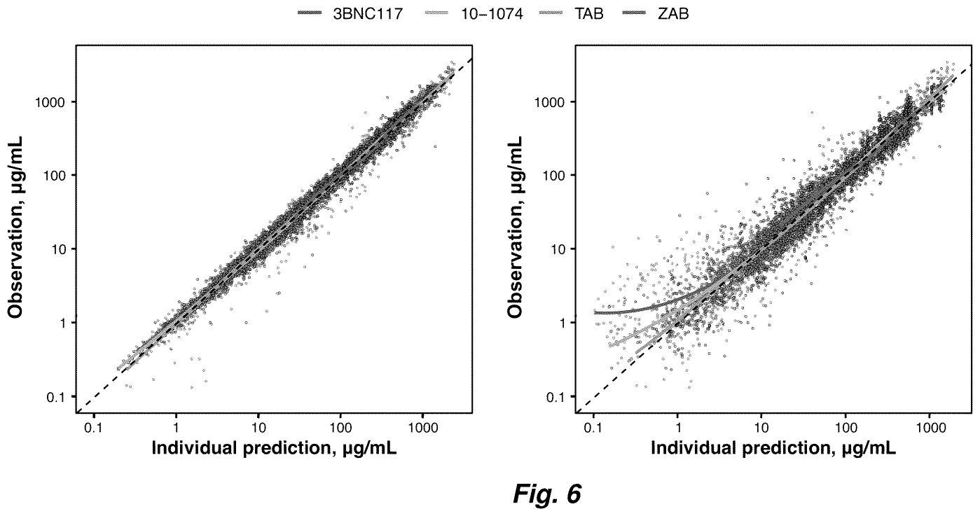

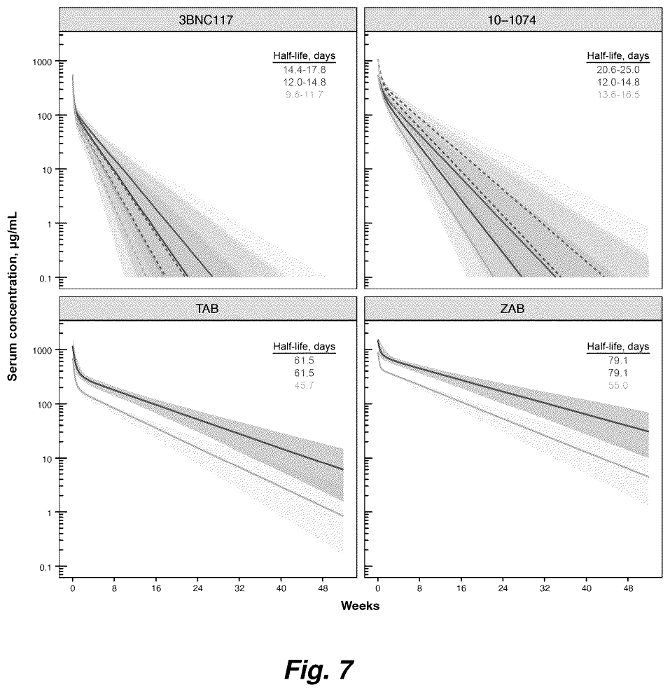

PK modeling. The PK data of 3BNC117, 10-1074, TAB, and ZAB were well described by linear 2-compartment PK models (FIG. 6). For 3BNC117 and 10-1074, the estimated half-lives were the longest in people without HIV, followed by suppressed people with HIV, and shortest in viremic people with HIV (FIG. 7). For TAB and ZAB, the estimated half-lives were longer than those of 3BNC117 and 10-1074, similar between people without HIV and suppressed people with HIV (62 and 79 days for TAB and ZAB, respectively), and shorter in viremic people with HIV (46 and 55 days for TAB and ZAB, respectively) (FIG. 7).

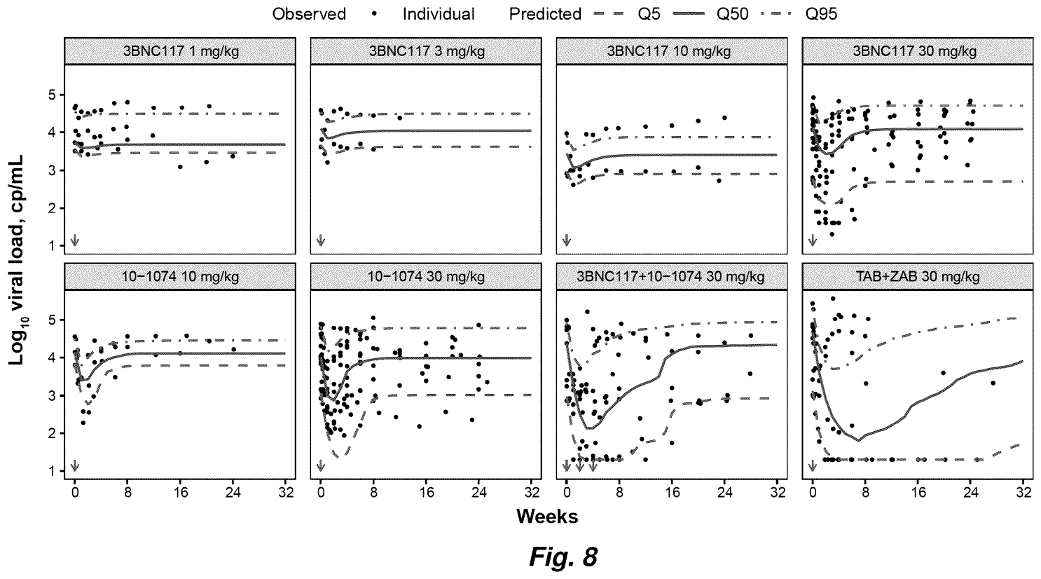

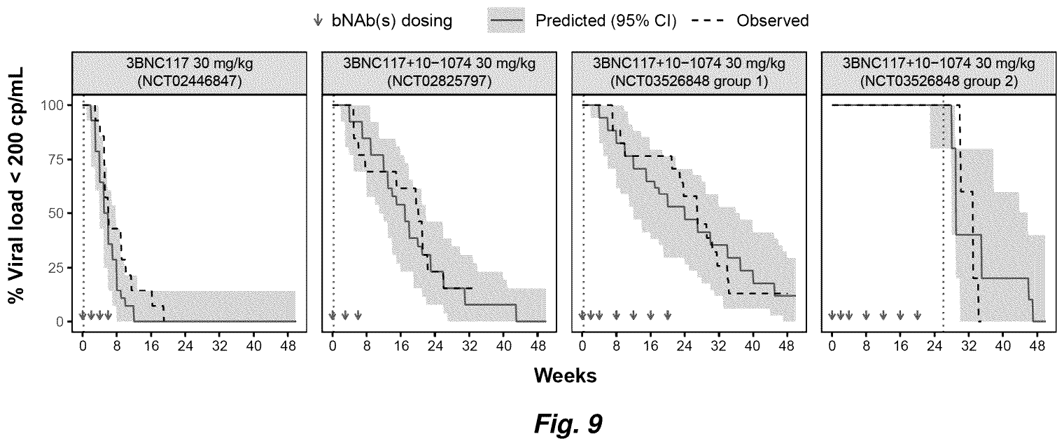

PK-PD modeling. The PK-PD model adequately described the dynamics of viral suppression in viremic people with HIV after bNAb treatment with 3BNC117, 10-1074 alone at different doses and in combination, as well as combination treatment with TAB and ZAB (FIG. 8). The model described the time to viral rebound during ATI after bNAb treatment with 3BNC117 alone or in combination with 10-1074 (FIG. 9). The estimated mean serum concentrations corresponding to 50% maximum drug effect (EC₅₀) of 3BNC117/TAB and 10-1074/ZAB were 25.4 and 32.2 μg/mL, which correspond to EC₂₀ of 6.35 and 8.06 μg/mL, respectively (Table 5).

TABLE 5
Key PK-PD model parameter estimates

| Parameter | Mean | 95% CI | % CV |
|---|---|---|---|
| EC_{50, 3BNC117 or TAB}, μg/mL^{a} | 25.4 | (19.6-32.9) | 162 |
| EC_{50, 10-1074 or ZAB}, μg/mL^{b} | 32.2 | (10.1-102.8 | 79.5 |
| Viral replication rate constant, kg, day⁻¹ | 0.441 | (0.414-0.468) | 24.5 |
| Viral elimination rate constant, k_{del, 3BNC117 or TAB}, day⁻¹ | 0.507 | (0.476-0.538) | — |
| Viral elimination rate constant, k _{del,} _{10-1074 or ZAB}, day⁻¹ | 0.799 | (0.609-0.990) | — |

CI, confidence interval; CV, coefficient of variation; EC₂₀, concentration that leads to 20% maximum drug effect; EC₅₀, concentration that leads to 50% maximum drug effect; PD, pharmacodynamic; PK, pharmacokinetic; TAB, teropavimab; ZAB, zinlirvimab.
^{a}Corresponds to mean (95% CI) EC₂₀ value of 6.35 (4.90-8.22) μg/mL.
^{b}Corresponds to mean (95% CI) EC20 value of 8.06 (2.53-25.7) μg/mL.

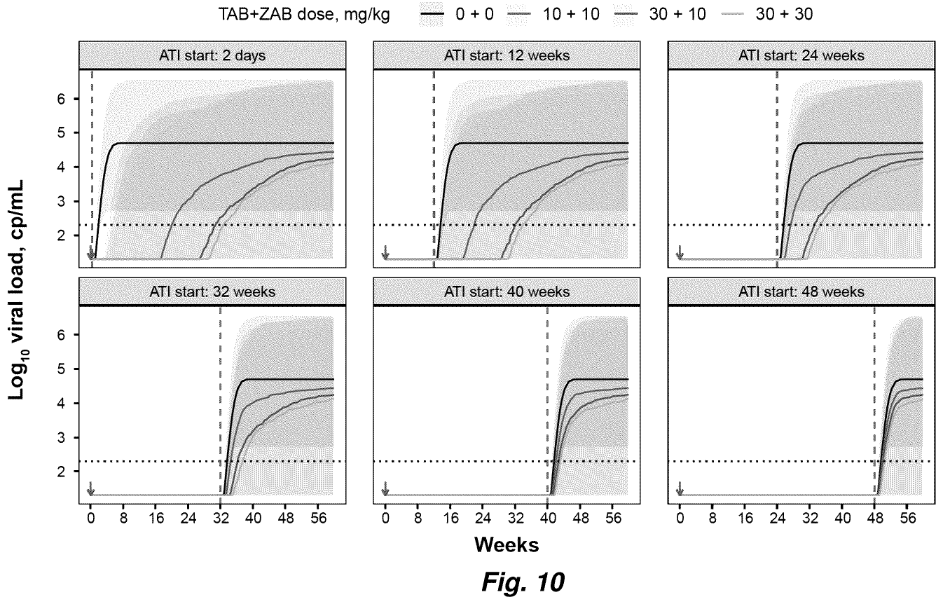

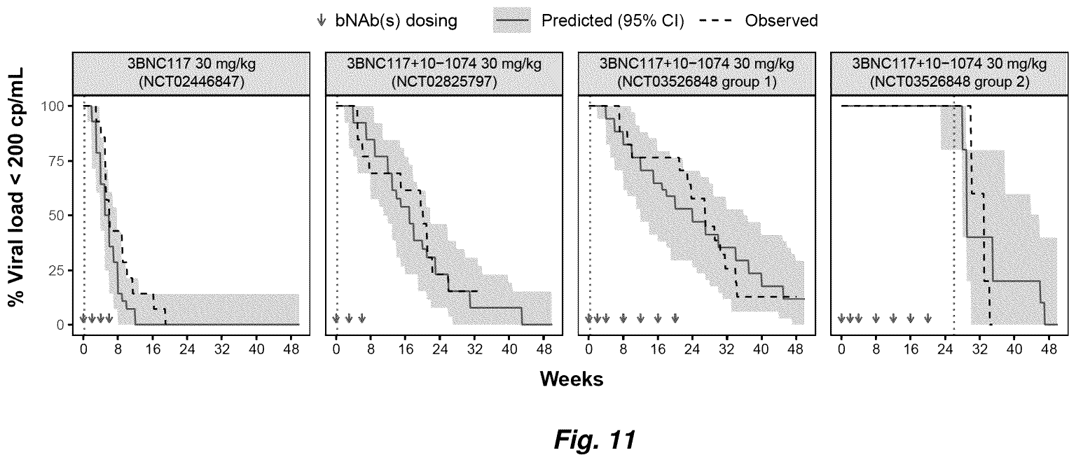

PK-PD stimulations. PK-PD simulations predicted that after a washout period of ≥48 weeks after single-dose TAB and ZAB intravenous administration, the viral neutralization effects of these bNAbs would have minimal impact on the time to viral rebound during ATI (FIG. 10). After single-dose 30 mg/kg TAB and 10 mg/kg ZAB intravenous administration, both bNAb concentrations were predicted to drop below their in vivo EC50 around similar times and maintain similar levels relative to the EC50 afterward, thus minimizing the risk of resistance development from functional monotherapy of either bNAb. At week 48, both bNAb concentrations were predicted to be lower than EC50 in over 90% of participants (FIG. 11).

### Example 4

### A Phase 2 Study of Teropavimab (GS-5423) and Zinlirvimab (GS-2872) in Combination with Capsid Inhibitor Lenacapavir (LEN) in Virologically Suppressed Adults with HIV-1 Infection

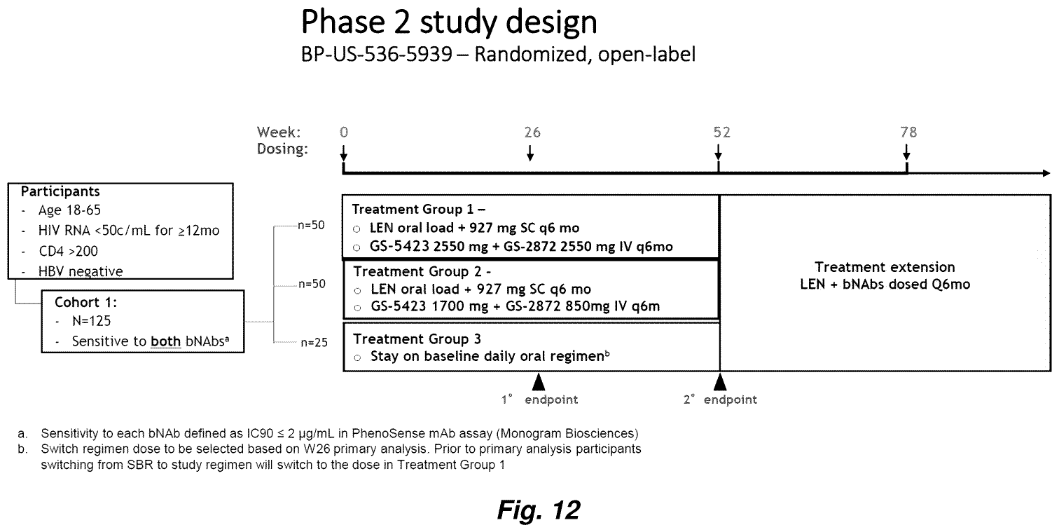

Study Design: GS-US-539-5939 (NCT05729568 on ClinicalTrials.gov) is a Phase 2, randomized, open-label, active-controlled, multicenter study to evaluate the safety and efficacy of the long-acting combination regimen of capsid inhibitor lenacapavir (LEN), teropavimab (GS-5423), and zinlirvimab (GS-2872). The study will include approximately 125 participants with sensitivity to both bNAbs by protocol-defined criteria, who meet all eligibility criteria, and will be randomized without stratification in a 2:2:1 ratio to Treatment Groups 1, 2, and 3. The clinical trial study schematic is depicted in FIG. 12.

Participants will take their last dose of baseline oral antiretroviral therapy (ART) on Day 1, participants randomized to Treatment Groups 1 and 2 will discontinue their baseline ART regimen following administration of the complete study regimen on Day 1 (subcutaneous injectable LEN, oral LEN 600 mg, and intravenous (IV) infusions of GS-5423 and GS-2872), and will self-administer oral LEN 600 mg on Day 2. Participants in Treatment Group 3 will continue their baseline oral ARV regimen as prescribed until Week 52. Participants randomized to Treatment Groups 1 and 2 will receive study drug (injectable LEN and IV infusions of GS-5423 and GS-2872) at Week 26. All participants in all Treatment Groups will return to the study center for visits at Weeks 4, 12, 24, 26, 38, 50, and 52.

At Week 52, participants in Treatment Groups 1 and 2 who received the study regimen of LEN, GS 5423, GS-2872, and completed study follow-up through Week 52 with plasma levels of HIV RNA less than (<) 50 copies/mL will be enrolled in the study extension phase. Participants who elect not to participate or not eligible to participate in the extension phase will resume their baseline ART regimen (or appropriate regimen selected by the investigator) and return for study follow-up visits at 30, 90, and 180 days post Week 52. Participants randomized to Treatment Group 3 who completed study follow-up through Week 52 with plasma levels of HIV-1 RNA <50 copies/mL throughout randomized phase of the study will receive the study regimen of LEN, GS-5423, and GS-2872 every 26 weeks. The dose of GS-5423 and GS-2872 will be determined at the time of the primary analysis. Participants in Treatment Group 3 who reach Week 52 prior to the primary analysis will receive the study regimen at the dose specified for Treatment Group 2 until after completion of the primary analysis and dose selection (unless Treatment Group 2 is modified in response to the data monitoring committee (DMC)). Participants in Treatment Group 3 who do not receive the study regimen after Week 52 will return for a 30-day follow-up visit.

An independent DMC will be convened to review safety and efficacy data at two planned interim analyses: after approximately the first 50% of participants enrolled have completed their Week 12 and 26 visits or prematurely discontinued from the study drug. In addition, if four or more participants in any LEN+bNAbs treatment group of any cohort experience virologic rebound (VR) before all participants reach Week 26, an ad hoc DMC meeting may be convened to assess the data.

Virologic Failure (VF): Participants experiencing virologic rebound (VR), as defined below, will be considered to be in a situation of virologic failure and may be subject to resistance analysis.

Virologic Rebound: Participants who meet the following criteria will be considered to have VR:
At any visit after Day 1, a rebound in HIV-1 RNA ≥50 copies/mL, which is subsequently confirmed at the following scheduled or unscheduled visit, or
Any participant with HIV-1 RNA ≥50 copies/mL at study drug discontinuation

If an above scheduled or ad-hoc interim DMC analysis of efficacy (based on virologic failure (VF), i.e., plasma levels of HIV-1 RNA greater than or equal to (≥) 50 copies/mL Weeks 12, 26, or virologic rebound crosses the futility boundary (i.e., lower bound of 95% confidence interval (CI) of treatment difference (Treatment Group 1 or Group 2—Stay on Baseline Regimen (SBR)) in proportion of VF >0) before all participants reach Week 26, DMC may recommend to drop an inferior dose arm. The decision to discontinue a dosing arm will be made by the Sponsor.

Target Population: Adults with HIV-1, on ART with demonstrated virologic suppression (plasma levels of HIV-1 RNA <50 copies/mL) for at least 12 months prior to screening and meeting protocol criteria for sensitivity to bNAbs.

Duration of Intervention: Up to 52 weeks during the randomized phase and 104 weeks during the extension phase.

TABLE 6
Test Product, Dose, and Mode of Administration:

| Treatment | | | | | | | | |
|---|---|---|---|---|---|---|---|---|
| Groups | Drug | | Day 1 | | Day 2 | | Week 26 | |
| 1 | Loading | LEN | 600 | mg PO* | 600 | mg PO | | |
| | Maintenance | LEN | 927 | mg SC | | | 927 | mg SC |
| | | GS-5423 | 2550 | mg IV | | | 2500 | mg IV |
| | | GS-2872 | 2550 | mg IV | | | 2550 | mg IV |
| 2 | Loading | LEN | 600 | mg PO | 600 | mg PO | | |
| | Maintenance | LEN | 927 | mg SC | | | 927 | mg SC |
| | | GS-5423 | 1700 | mg IV | | | 1700 | mg IV |
| | | GS-2872 | 850 | mg IV | | | 850 | mg IV |

*PO = Per Os, oral administration; SC = subcutaneous; IV = intravenous

Statistical Methods: The primary efficacy endpoint is the proportion of participants with HIV-1 RNA ≥50 copies/mL at Week 26 as defined by the FDA-defined snapshot algorithm. The 95% CIs will be constructed using the unconditional exact method. The efficacy endpoint will be compared between treatment groups by Fisher exact test. The proportion of participants with HIV 1 RNA ≥50 copies/mL at Week 52 and the proportion of participants with HIV-1 RNA <50 copies/mL at Weeks 26 and 52 as determined by the US FDA-defined snapshot algorithm will be analyzed using the same methods as for the primary efficacy endpoint.

The changes from baseline in CD4+ T-cell count will be summarized by treatment using descriptive statistics. The differences in changes from baseline in CD4+ T-cell count between the 2 treatments groups will be compared.

Treatment-emergent adverse events (AEs), serious adverse events (SAEs), and adverse events leading to permanent study drug discontinuation will be summarized by treatment group, system organ class (SOC), and preferred term using the current version of the Medical Dictionary for Regulatory Activities (MedDRA). Laboratory results and change from baseline values for selected laboratory tests will be summarized by treatment group and visit. The incidence of treatment-emergent laboratory abnormalities will be summarized by treatment group. Vital signs and electrocardiogram data will be summarized by treatment group.

Serum or plasma concentrations and PK parameters for GS-5423, GS-2872, and LEN (and metabolites, if applicable) will be listed and summarized for each analyte using descriptive statistics by treatment group, as appropriate.

TABLE 7
Objectives and Endpoints

| Primary Objective(s) | Primary End Point(s) |
|---|---|
| To evaluate the efficacy of the study | Proportion of participants with HIV-1 |
| regimens as determined by the | RNA ≥50 copies/mL at Week 26 as |
| proportion of participants with virologic | determined by the United States (US) |
| rebound (HIV-1 RNA ≥50 copies/mL) at | Food and Drug Administration (FDA)- |
| Week 26 | defined snapshot algorithm |
| Secondary Objective(s) | Secondary End Point(s) |
| To evaluate the efficacy of the study | Proportion of participants with HIV-1 |
| regimens as determined by the | RNA ≥50 copies/mL at Week 52 as |
| proportion of participants with virologic | determined by the US FDA-defined |
| rebound (HIV-1 RNA ≥50 copies/mL) at | snapshot algorithm |
| Week 52 | Proportion of participants with HIV-1 |
| To evaluate the efficacy of the study | RNA <50 copies/mL at Weeks 26, and |
| regimens as determined by the | 52 as defined by the US FDA-defined |
| proportion of participants maintaining | snapshot algorithm |
| virologic suppression (HIV-1 RNA <50 | Changes from baseline in CD4+ T-cell |
| copies/mL) at Weeks 26, and 52 | counts at Weeks 26 and 52 |
| To evaluate CD4+ T-cell counts at | Proportion of participants experiencing |
| Weeks 26, and 52 | treatment-emergent adverse events |
| To evaluate the safety and tolerability of | (TEAEs) |
| the study regimen through 26 and 52 | PK parameters for GS-5423, GS-2872, |
| Weeks | and LEN as appropriate: AUC₀₋ₜ, |
| To evaluate the pharmacokinetics (PK) | AUCₗₐₛₜ, t_{1/2}, Cₘₐₓ, Tₘₐₓ |
| of GS-5423, GS-2872, and lenacapavir | Proportion of participants who develop |
| (LEN) | anti-GS-5423 and/or anti-GS-2872 |
| To evaluate the immunogenicity of GS- | antibodies through Weeks 26 and 52 |
| 5423 and GS-2872 | |
| Exploratory Objective(s) | Exploratory End Point(s) |
| To evaluate the emergence of viral | Treatment-emergent viral resistance to |
| resistance during study treatment | study drugs through Week 52 |
| To evaluate changes in the HIV reservoir | Changes from baseline in HIV-1 reservoir |
| To evaluate the effect of every 6-month | in peripheral blood mononuclear cells |
| bNAbs/LEN treatment on patient- | (PBMCs) |
| reported outcomes | HIV-TSQ and treatment preference |
| | questionnaires |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-39. (canceled)

40. A method of treating or preventing HIV in a human subject in need thereof, the method comprising:
a) Co-administering at a first time point (i) an effective amount of 10-1074-LS (zinlirvimab; GS-2872) and (ii) an effective amount of 3BNC117-LS (teropavimab; (GS-5423)); and
b) Co-administering at a second time point at least about 24 weeks, e.g., at least about 25 weeks, e.g., at least about 26 weeks, after the first time point an effective amount of 10-1074-LS and an effective amount of 3BNC117-LS.

41. The method of claim 40, wherein the 10-1074-LS and the 3BNC117-LS are co-administered every 6 months (Q6M).

42. The method of claim 40, wherein the 10-1074-LS and the 3BNC117-LS are co-administered every 24 weeks (Q24W).

43. The method of claim 40, wherein the 10-1074-LS and the 3BNC117-LS are co-administered every 25 weeks (Q25W).

44. The method of claim 40, wherein the 10-1074-LS and the 3BNC117-LS are co-administered every 26 weeks (Q26W).

45. The method of claim 40, wherein the 10-1074-LS and the 3BNC117-LS are co-administered 2 times over 1 year.

46. The method of claim 40, wherein the 10-1074-LS and the 3BNC117-LS are co-administered 4 times over 2 years.

47. The method of claim 40, wherein the 10-1074-LS and the 3BNC117-LS are co-administered 6 times over 3 years.

48. The method of claim 40, wherein the 10-1074-LS and the 3BNC117-LS are co-administered 8 times over 4 years.

49. The method of claim 40, wherein the 10-1074-LS is administered intravenously at a dose of 30 mg/kg and the 3BNC117-LS is administered intravenously at a dose of 30 mg/kg.

50. The method of claim 40, wherein the 10-1074-LS is administered intravenously at a dose of 10 mg/kg and the 3BNC117-LS is administered intravenously at a dose of 30 mg/kg.

51. The method of claim 40, wherein the 10-1074-LS and the 3BNC117 are independently administered intravenously at a dose in the range of from about 500 mg to about 3000 mg.

52. The method of claim 51, wherein the 10-1074-LS is administered intravenously at a dose of 2550 mg and the 3BNC117-LS is administered intravenously at a dose of 2550 mg.

53-54. (canceled)

55. The method of claim 51, wherein the 10-1074-LS is administered intravenously at a dose of 850 mg and the 3BNC117-LS is administered intravenously at a dose of 2550 mg.

56. The method of claim 40, wherein the serum concentration of the 10-1074-LS and the 3BNC117-LS are at least 10 μg/mL at 26 weeks after the first time point.

57. The method of claim 40, wherein the plasma or serum concentration of HIV RNA is less than 50 copies/mL at 26 weeks after the first time point.

58. The method of claim 40, further comprising co-administering one or more long-acting HIV drugs.

59. The method of claim 58, one or more long-acting HIV drugs are selected from a long-acting capsid inhibitor, a long-acting integrase strand transfer inhibitor (INSTI), a long-acting non-nucleoside reverse transcriptase inhibitor (NNRTI), a long-acting nucleoside reverse transcriptase inhibitors (NRTI), and a long-acting protease inhibitor (PI).

60. The method of claim 59, wherein the long-acting capsid inhibitor is selected from lenacapavir, VH4004280 and VH4011499.

61. The method of claim 59, wherein the long-acting capsid inhibitor comprises lenacapavir.

62. The method of claim 61, wherein the lenacapavir is administered at a dose in the range of 300 mg to 1000 mg.

63. The method of claim 60, wherein the lenacapavir is administered orally or subcutaneously.

64. The method of claim 59, wherein the long-acting INSTI is selected from bictegravir, raltegravir, elvitegravir, dolutegravir, cabotegravir, GS-1720, GS-6212, GS-1219, GS-3242 and VH4524184.

65. The method of claim 59, wherein the long-acting NNRTI is selected from rilpivirine, elsulfavirine, doravirine and GS-5894.

66. The method of claim 59, wherein the long-acting NRTI is selected from islatravir and prodrugs thereof, tenofovir alafenamide (TAF) and prodrugs of tenofovir, rovafovir etalafenamide and GS-1614.

67. The method of claim 59, wherein the long-acting protease inhibitor is selected from atazanavir, ritonavir, darunavir, GS-1156 and prodrugs of GS-1156, and combinations thereof.

68. The method of claim 40, further comprising determining the sensitivity of the HIV in the subject to one or both of 10-1074-LS and 3BNC117-LS.

69. The method of claim 40, wherein the subject is heavily treatment experienced (HTE).

70. The method of claim 40, wherein the subject is resistant or non-responsive to one or more of an integrase strand transfer inhibitor (INSTI), a non-nucleoside reverse transcriptase inhibitor (NNRTI), a nucleoside reverse transcriptase inhibitors (NRTI), and a protease inhibitor (PI).

71. The method of claim 40, wherein the subject is viremic.

72. The method of claim 40, wherein the subject is virologically suppressed.

73. The method of claim 40, wherein the subject is receiving antiretroviral therapy (ART).

74. The method of claim 40, wherein antiretroviral therapy (ART) has been discontinued before administration of 10-1074-LS and 3BNC117-LS.

75. The method of claim 40, wherein the subject is acutely infected with HIV.

76. The method of claim 75, wherein the subject has an HIV infection of Fiebig stage IV or earlier.

77. The method of claim 76, wherein the subject has not seroconverted.

78. The method of claim 40, wherein the subject is recently infected with HIV.

79. The method of claim 78, wherein the antibody is administered to a subject having an HIV infection of Fiebig stage V or Fiebig stage VI.

80. The method of claim 40, wherein the subject is chronically infected with HIV.

81. The method of claim 40, wherein the subject is infected with HIV clade B viruses.

82. A kit comprising one or more unitary doses of a first antibody that binds HIV gp120 V3 glycan and a second antibody that binds HIV gp120 CD4bs, wherein the first antibody and the second antibody have serum half-life extending amino acid substitutions, and wherein the first antibody and the second antibody are formulated for administration twice annually (e.g., every 6 months (Q6M), every 26 weeks (Q26W), every 25 weeks (Q25W), or every 24 weeks (Q24W)).

83-102. (canceled)
